(19) United States
(12) Patent Application Publication
(10) Pub. No.: US 2023/0398110 A1
(43) Pub. Date: Dec. 14, 2023

(54) COMPOSITIONS AND METHODS FOR TREATING NON-ALCOHOLIC STEATOHEPATITIS

(71) Applicants: ASCENTAGE PHARMA (SUZHOU) CO., LTD., SUZHOU (CN); ASCENTAGE PHARMA GROUP CORP LIMITED, CENTRAL (CN)

(72) Inventors: Yifan ZHAI, Suzhou (CN); Dajun YANG, Suzhou (CN); Douglas Dong FANG, Suzhou (CN); Qiang LI, Suzhou (CN)

(21) Appl. No.: 18/006,494

(22) PCT Filed: Aug. 20, 2021
(86) PCT No.: PCT/CN2021/113826

§ 371 Date: Jan. 23, 2023

(30) Foreign Application Priority Data
Aug. 21, 2020 (WO) PCT/CN2020/110441

(51) Int. Cl. A61K 31/496; A61P 1/14; A61K 31/575; A61P 29/00

(52) U.S. Cl. CPC A61K 31/496; A61P 29/00; A61K 31/575; A61P 1/14

(57) ABSTRACT

Methods for treating non-alcoholic steatohepatitis using compounds or pharmaceutical compositions that modulate the activity of Bcl-2 family proteins are disclosed. In some methods, the patient to be treated is diagnosed with one or more additional diseases selected from cardiovascular disease, chronic kidney disease, type 2 diabetes mellitus, obesity, and metabolic syndrome, wherein the metabolic syndrome may include, but is not limited to patient presentation of one or more of hypertension, hyperglycaemia, hyper-lipemia, insulin resistance (IR). In some methods, the compound or pharmaceutical composition is administered to the patient in need thereof at a therapeutically effective dose sufficient to elicit one or more effects selected from: reduced liver steatosis, reduced lobular inflammation, reduced hepatocellular ballooning, and reduced liver fibrosis.

### TECHNICAL FIELD

The present disclosure relates to methods of treating non-alcoholic steatohepatitis (NASH) using Bcl-2 inhibitor compounds or compositions comprising the same.

### BACKGROUND

Apoptosis, the process of programmed cell death, is an essential biological process for tissue homeostasis. In mammals, it has been shown to regulate early embryonic development. Later in life, cell death is a default mechanism by which potentially dangerous cells, e.g., cells carrying cancerous defects, are removed. Several apoptotic pathways are known. One of the most important apoptotic pathways involves the Bcl-2 family of proteins which are key regulators of the mitochondrial (also called “intrinsic”) pathway of apoptosis. See Danial and Korsmeyer, Cell 116:205-219 (2004). The structural homology domains BH1, BH2, BH3 and BH4 are characteristic of Bcl-2 family proteins. The Bcl-2 family of proteins can be further classified into three subfamilies depending on how many of the homology domains each protein contains and on its biological activity, i.e., whether it has pro- or anti-apoptotic function.

The first subgroup of Bcl-2 proteins contains proteins having all four homology domains, i.e., BH1, BH2, BH3 and BH4. Their general effect is anti-apoptotic, that is to preserve a cell from starting a cell death process. Proteins such as Bcl-2, Bcl-w, Bcl-xL, Mcl-1, and Bfl-1/Al are members of this first subgroup. Proteins belonging to the second subgroup of Bcl-2 proteins contain the three homology domains BH1, BH2, and BH3, and have a pro-apoptotic effect. The two main representative proteins of this second subgroup are Bax and Bak. The third subgroup of Bcl-2 proteins is composed of proteins containing only the BH3 domain and members of this subgroup are usually referred to as “BH3-only proteins.” Their biological effect on the cell is pro-apoptotic. Bim, Bid, Bad, Bik, Noxa, Hrk, Bmf, and Puma are examples of this third subfamily of proteins. The exact mechanism by which the Bcl-2 family proteins regulate cell death is not entirely known. In one hypothesis of regulation of cell death by Bcl-2 family proteins, the BH3-only proteins are further categorized as either “activator,” e.g., Bim and Bid, or “sensitizer,” e.g., Bad, Bik, Noxa, Hrk, Bmf, and Puma, proteins depending on their regulatory function.

One of the keys to tissue homeostasis is achieving a balance in the interactions among the three subgroups of Bcl-2 proteins in cells. Studies have elucidated the mechanisms by which pro-apoptotic and anti-apoptotic subgroups of Bcl-2 family proteins interact to allow a cell to undergo programmed cell death. After receiving intra- or extracellular signals in cells, post-translational or transcriptional activation of BH3-only proteins occurs. The BH3-only proteins are the primary inducers of an apoptotic cascade that includes, as one step, the activation of the pro-apoptotic proteins Bax and Bak on the mitochondrial membrane in cells. Upon activation of Bax and/or Bak that are either already anchored to the mitochondrial membrane or migrate to this membrane, Bax and/or Bak oligomerize to result in mitochondrial outer membrane permeabilization (MOMP), the release of cytochrome C, and downstream activation of effector caspases, to ultimately result in cell apoptosis. Some researchers hypothesize that certain BH3-only proteins, e.g., Puma, Bim, Bid, are “activators” in that these proteins directly engage pro-apoptotic proteins Bax and Bak to initiate MOMP, while other BH3-only proteins, e.g., Bad, Bik and Noxa, are “sensitizers” and induce Bax and Bak oligomerization indirectly by binding anti-apoptotic proteins, e.g., Bcl-2, Bcl-xL, Bcl-w, Mcl-1, and displacing and “freeing-up” the “activator” BH3-only proteins, which subsequently bind to and activate pro-apoptotic proteins, e.g., Bax, Bak, to induce cell death. Other research suggests that anti-apoptotic proteins engage and sequester Bax and Bak directly and all BH3-only proteins regulates this interaction by binding to anti-apoptotic proteins, e.g., Bcl-2, Bcl-xL, Bcl-w, Mcl-1, which results in the release Bax and Bak. See Adams and Cory, Oncogene 26:1324-1337 (2007) and Willis et al., Science 315:856-859 (2007). Although the exact interactions through which the anti- and pro-apoptotic Bcl-2 family proteins regulate apoptosis remain under investigation, there is a large body of scientific evidence to show that compounds which inhibit the binding of BH3-only proteins to anti-apoptotic Bcl-2 family proteins promote apoptosis in cells.

Dysregulated apoptotic pathways have been implicated in the pathology of many significant diseases such as neurodegenerative conditions (up-regulated apoptosis), such as for example, Alzheimer's disease; and proliferative diseases (down-regulated apoptosis) such as for example, cancer, autoimmune diseases and pro-thrombotic conditions.

For example, defects in Bcl-2 signaling have been reported to cause or correlate with autoimmunity in mouse models and human patients. In particular, proper Bcl-2 signaling is critical to B-cell development, and dysregulated Bcl-2 signaling is linked to the loss of self-tolerance and the development of autoimmune disorders (Tischner et al. (2010), Cell Death Dis. 1 (6):e48). Accordingly, Bcl-2 inhibitor compounds may offer an attractive therapeutic strategy for the restoration of normal Bcl-2 signaling in the treatment of autoimmune diseases and other B-cell associated disorders.

B-cells have additionally been linked to the development of liver fibrosis following liver damage via an antibody-independent mechanism. For example, mouse model studies of carbon tetrachloride-induced liver damage demonstrated that B-cell deficient mice had significantly less collagen deposition in the liver in comparison to wild-type mice, which is a hallmark of liver fibrosis development (Novobrantseva et al. (2005), J. Clin. Invest. 115:3072-3082). Thus, Bcl-2 inhibitors may be useful in the treatment of diseases associated with liver fibrosis through the restoration of normal B-cell regulation, such as non-alcoholic steatohepatitis.

Accordingly, there is an ongoing need for small molecules that selectively inhibit the activity of one type or a subset of Bcl-2 proteins for the treatment of non-alcoholic steatohepatitis.

### SUMMARY

Disclosed herein are methods of treating non-alcoholic steatohepatitis in a patient comprising administering to the patient in need thereof a therapeutically effective amount of a compound of formula (I):

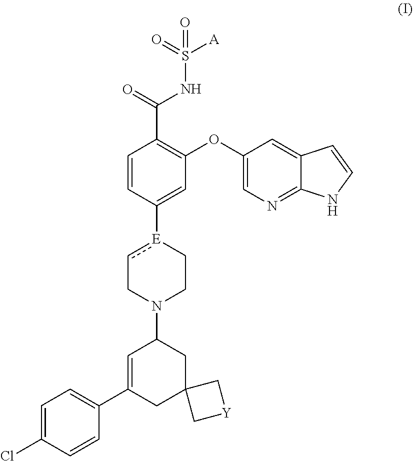

or a pharmaceutically acceptable salt, hydrate, solvate, stereoisomer, or tautomer thereof, wherein A, E, and Y are as defined herein.

Also disclosed herein are methods of treating non-alcoholic steatohepatitis in a patient comprising administering to the patient in need thereof a therapeutically effective amount of a pharmaceutical composition comprising a compound of formula (I), as depicted above, or a pharmaceutically acceptable salt, hydrate, solvate, stereoisomer, or tautomer thereof.

In one embodiment, a method of treating non-alcoholic steatohepatitis in a patient comprises administering to the patient in need thereof a therapeutically effective amount of a compound of formula (I):

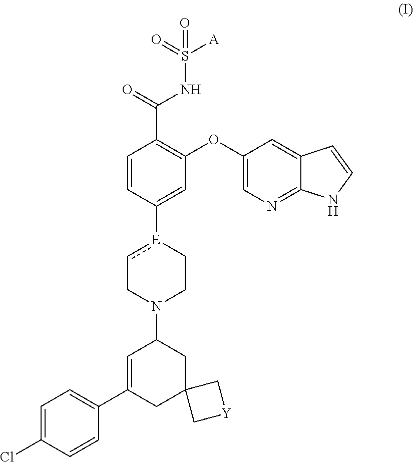

or a pharmaceutically acceptable salt, solvate, hydrate, stereoisomer, or tautomer thereof wherein:

A is selected from the group consisting of:

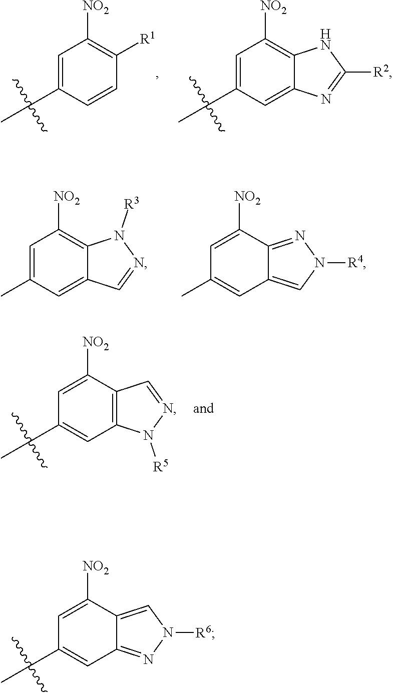

E is selected from the group consisting of:
a carbon atom, wherein [symbol] is a double bond;
—C(H)—, wherein [symbol] is a single bond; and
a nitrogen atom, wherein [symbol] is a single bond;

Y is selected from —C(H)— and —O—;

R¹ is selected from hydrogen and —N(R^{7a})(R^{7b});

R², R³, R⁴, R⁵, and R⁶ are each independently selected from the group consisting of hydrogen, optionally substituted C₁₋₆ alkyl, optionally substituted C₃₋₆ cycloalkyl, heterocyclo, optionally substituted heteroaryl, (heterocyclo)alkyl;

R^{7a} is selected from optionally substituted C₁₋₆ alkyl and optionally substituted (heterocyclo)alkyl; and

R^{7b} is selected from hydrogen and C₁₋₄ alkyl.

In one embodiment, the method comprises administering to the patient in need thereof a therapeutically effective amount of a compound of formula (I) that is further given by formula (II):

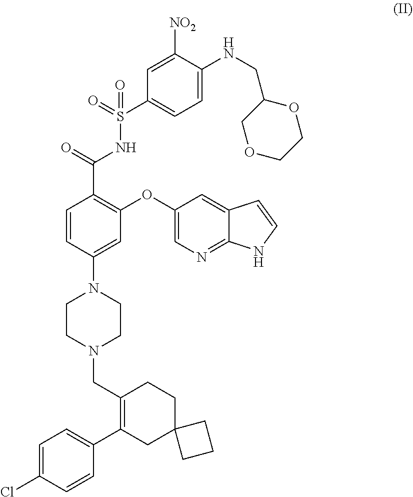 [SMILES: O=C(NS(=O)(=O)c1ccc(NCC2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, stereoisomer, or tautomer thereof.

In one embodiment, the method comprises administering to the patient in need thereof a therapeutically effective amount of a compound selected from the group consisting of:

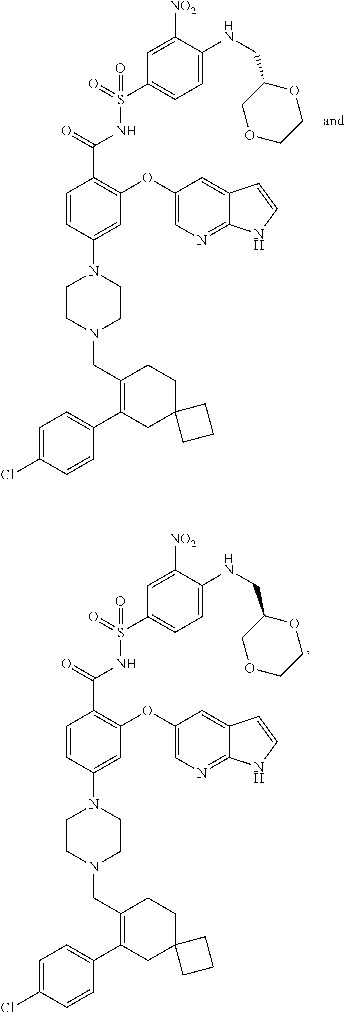 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the method comprises administering to the patient in need thereof a therapeutically effective amount of a compound, wherein the compound is:

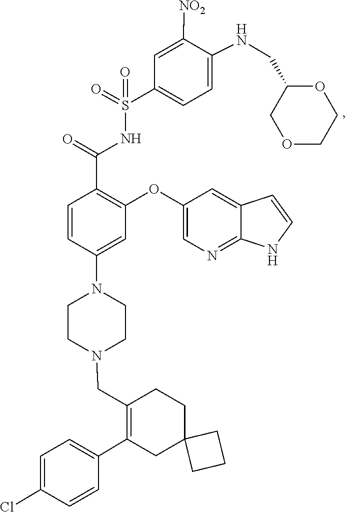 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the method comprises administering to the patient in need thereof a therapeutically effective amount of a compound, wherein the compound is:

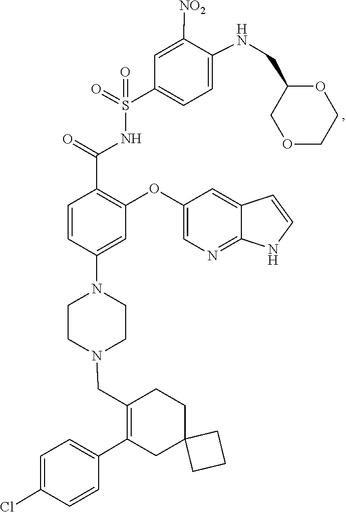 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the method comprises administering to the patient in need thereof a therapeutically effective amount of a compound selected from a group consisting of the compounds recited in Table 1, as disclosed herein.

In one embodiment, a method of treating non-alcoholic steatohepatitis in a patient comprises administering to the patient in need thereof a therapeutically effective amount of a pharmaceutical composition comprising a compound of formula (I):

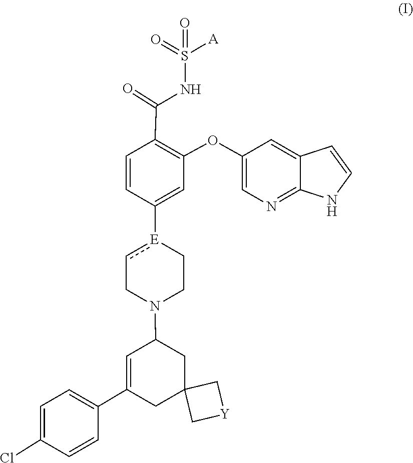

or a pharmaceutically acceptable salt, solvate, hydrate, stereoisomer, or tautomer thereof wherein:

A is selected from the group consisting of:

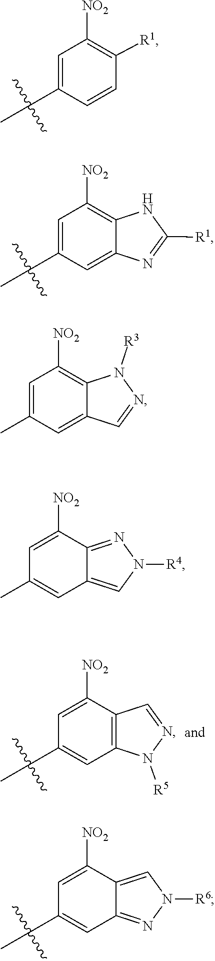

E is selected from the group consisting of:
a carbon atom, wherein [symbol] is a double bond;
—C(H)—, wherein [symbol] is a single bond; and
a nitrogen atom, wherein [symbol] is a single bond;

Y is selected from —C(H)— and —O—;

R¹ is selected from hydrogen and —N(R^{7a})(R^{7b});

R², R³, R⁴, R⁵, and R⁶ are each independently selected from the group consisting of hydrogen, optionally substituted C₁₋₆ alkyl, optionally substituted C₃₋₆ cycloalkyl, heterocyclo, optionally substituted heteroaryl, (heterocyclo)alkyl;

R^{7a} is selected from optionally substituted C₁₋₆ alkyl and optionally substituted (heterocyclo)alkyl; and

R^{7b} is selected from hydrogen and C₁₋₄ alkyl.

In one embodiment, the method comprises administering to the patient in need thereof a therapeutically effective amount of a pharmaceutical composition comprising a compound of formula (I) that is further given by formula (II):

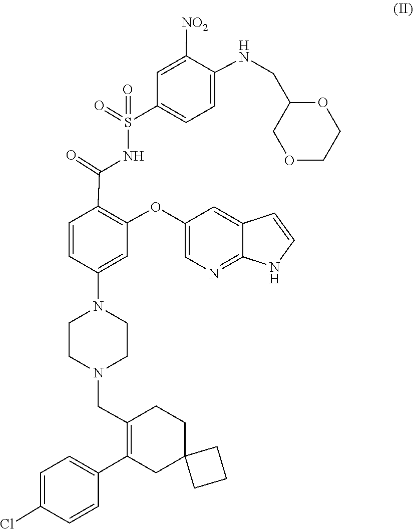 [SMILES: O=C(NS(=O)(=O)c1ccc(NCC2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, stereoisomer, or tautomer thereof.

In one embodiment, the method comprises administering to the patient in need thereof a therapeutically effective amount of a pharmaceutical composition comprising a compound selected from:

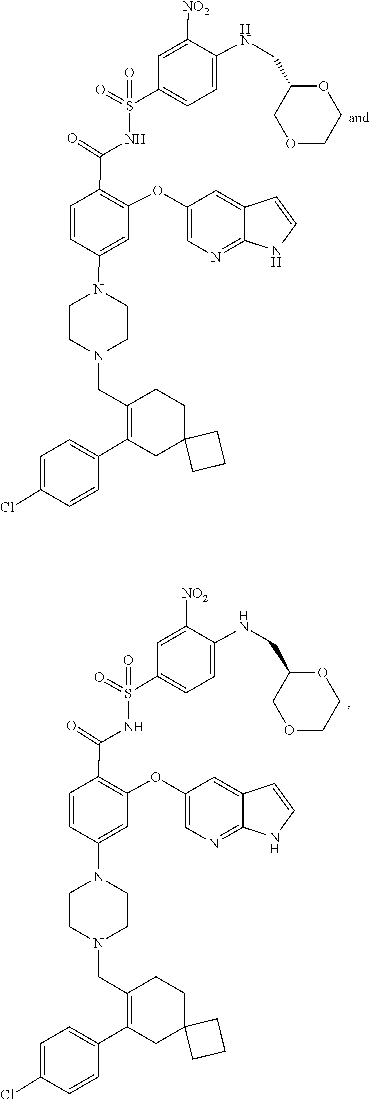 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the method comprises administering to the patient in need thereof a therapeutically effective amount of a pharmaceutical composition comprising

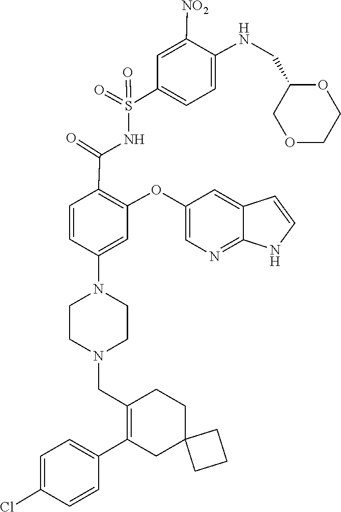 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the method comprises administering to the patient in need thereof a therapeutically effective amount of a pharmaceutical composition comprising

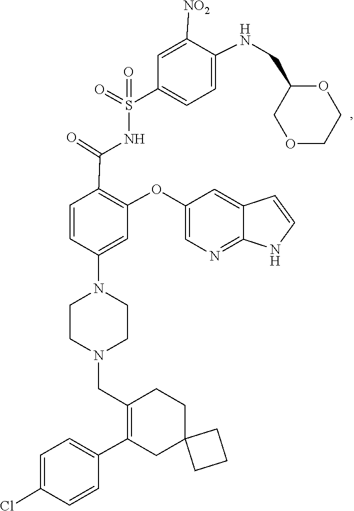 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the method comprises administering to the patient in need thereof a therapeutically effective amount of a pharmaceutical composition comprising a compound selected from a group consisting of the compounds recited in Table 1, as described herein.

In one embodiment, a method of treating non-alcoholic steatohepatitis in a patient comprises administering to the patient in need thereof a therapeutically effective amount of a compound, wherein the compound is (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide.

In one embodiment, the patient in need thereof is diagnosed as having one or more diseases selected from cardiovascular disease, chronic kidney disease, type 2 diabetes mellitus, obesity, and metabolic syndrome, wherein the metabolic syndrome is selected from hyperglycaemia, hyperlipemia, and insulin resistance (IR).

In one embodiment, the compound or pharmaceutical composition is administered to the patient in need thereof at a dose sufficient to elicit one or more effects selected from the group consisting of: reduced liver steatosis, reduced lobular inflammation, reduced hepatocellular ballooning, and reduced liver fibrosis.

In one embodiment, the compound or pharmaceutical composition is administered to the patient in need thereof at a dose sufficient to reduce liver steatosis in the patient.

In one embodiment, the compound or pharmaceutical composition is administered to the patient in need thereof at a dose sufficient to reduce lobular inflammation in the patient.

In one embodiment, the compound or pharmaceutical composition is administered to the patient in need thereof at a dose sufficient to hepatocellular ballooning in the patient.

In one embodiment, the compound or pharmaceutical composition is administered to the patient in need thereof at a dose sufficient to reduce liber fibrosis in the patient.

In one embodiment, the method further comprises administering to the patient in need thereof a therapeutically effective amount of obeticholic acid.

In one embodiment, the obeticholic acid is administered before the compound of formula (I).

In one embodiment, the obeticholic acid is administered concurrently with the compound of formula (I).

In one embodiment, the obeticholic acid is administered after the compound of formula (I).

In one embodiment, a compound of formula (I):

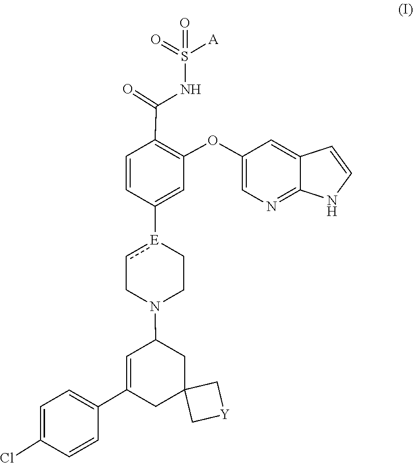

or a pharmaceutically acceptable salt, solvate, hydrate, stereoisomer or tautomer thereof, is used in the manufacture of a medicament for treating non-alcoholic steatohepatitis in a patient in need thereof, wherein:

A is selected from the group consisting of:

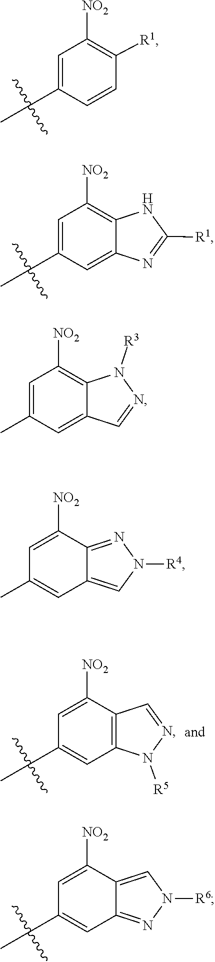

E is selected from the group consisting of:
a carbon atom, wherein [symbol] is a double bond;
—C(H)—, wherein [symbol] is a single bond; and
a nitrogen atom, wherein [symbol] is a single bond;

Y is selected from —C(H)— and —O—;

R¹ is selected from hydrogen and —N(R^{7a})(R^{7b});

R², R³, R⁴, R⁵, and R⁶ are each independently selected from the group consisting of hydrogen, optionally substituted C₁₋₆ alkyl, optionally substituted C₃₋₆ cycloalkyl, heterocyclo, optionally substituted heteroaryl, (heterocyclo)alkyl;

R^{7a} is selected from optionally substituted C₁₋₆ alkyl and optionally substituted (heterocyclo)alkyl; and

R^{7b} is selected from hydrogen and C₁₋₄ alkyl.

In one embodiment, the compound for use in the manufacture of the medicament is a compound of formula (I) that is further given by formula (II):

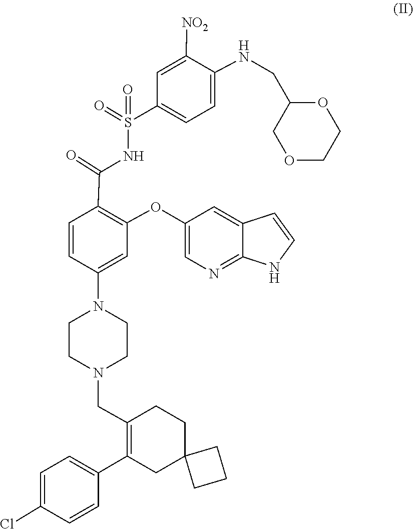 [SMILES: O=C(NS(=O)(=O)c1ccc(NCC2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, stereoisomer, or tautomer thereof.

In one embodiment, the compound for use in the manufacture of the medicament is selected from the group consisting of:

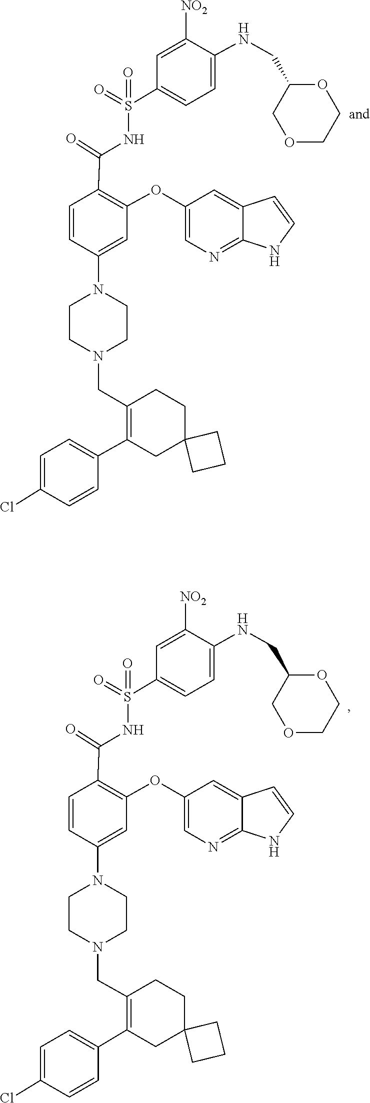 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the compound for use in the manufacture of the medicament is:

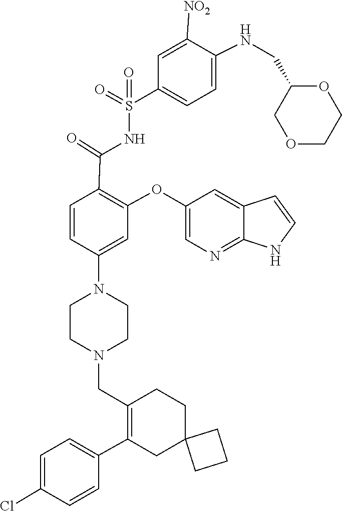 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the compound for use in the manufacture of the medicament is:

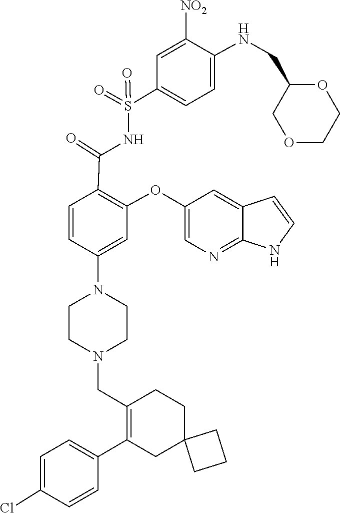 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the compound for use in the manufacture of the medicament is selected from a group of compounds consisting of the compounds recited in Table 1, as described herein.

In one embodiment, (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide is used in the manufacture of a medicament for treating non-alcoholic steatohepatitis in a patient in need thereof.

In one embodiment, the medicament is for treating non-alcoholic steatohepatitis in a patient diagnosed as having cardiovascular disease, chronic kidney disease, type 2 diabetes mellitus, obesity, and metabolic syndrome. wherein the metabolic syndrome is selected from hypertension, hyperglycaemia, hyperlipemia, and insulin resistance (IR).

In one embodiment, the compound or pharmaceutical composition is used for the manufacture of a medicament for treating non-alcoholic steatohepatitis, wherein the compound or pharmaceutical composition is administered to the patient in need thereof at a dose sufficient to elicit one or more effects selected from the group consisting of: reduced liver steatosis, reduced lobular inflammation, reduced hepatocellular ballooning, and reduced liver fibrosis.

In one embodiment, the compound or pharmaceutical composition is used for the manufacture of a medicament for treating non-alcoholic steatohepatitis, wherein the compound or pharmaceutical composition is administered to the patient in need thereof at a dose sufficient to reduce liver steatosis in the patient.

In one embodiment, the compound or pharmaceutical composition is used for the manufacture of a medicament for treating non-alcoholic steatohepatitis, wherein the compound or pharmaceutical composition is administered to the patient in need thereof at a dose sufficient to reduce lobular inflammation in the patient.

In one embodiment, the compound or pharmaceutical composition is used for the manufacture of a medicament for treating non-alcoholic steatohepatitis, wherein the compound or pharmaceutical composition is administered to the patient in need thereof at a dose sufficient to reduce hepatocellular ballooning in the patient.

In one embodiment, the compound or pharmaceutical composition is used for the manufacture of a medicament for treating non-alcoholic steatohepatitis, wherein the compound or pharmaceutical composition is administered to the patient in need thereof at a dose sufficient to reduce the severity of liver fibrosis in the patient.

In one embodiment, a compound for use in the treatment of systemic non-alcoholic steatohepatitis in a patient in need thereof comprises a compound of formula (I):

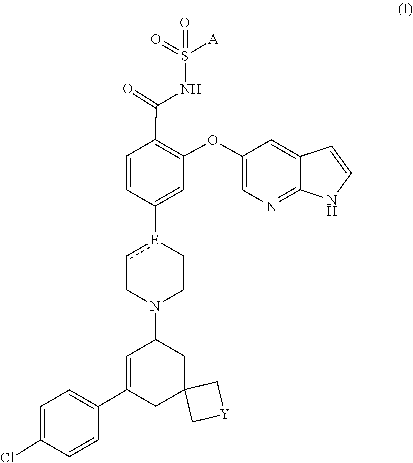

or a pharmaceutically acceptable salt, solvate, hydrate, stereoisomer, or tautomer thereof, wherein:

A is selected from the group consisting of:

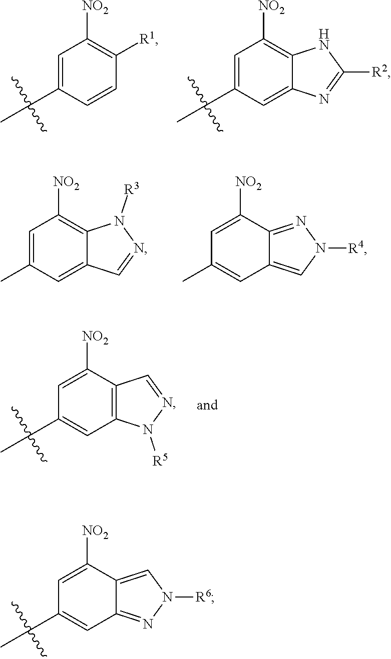

E is selected from the group consisting of:
a carbon atom, wherein [symbol] is a double bond;
—C(H)—, wherein [symbol] is a single bond; and
a nitrogen atom, wherein [symbol] is a single bond;

Y is selected from —C(H)— and —O—;

R¹ is selected from hydrogen and —N(R^{7a})(R^{7b});

R², R³, R⁴, R⁵, and R⁶ are each independently selected from the group consisting of hydrogen, optionally substituted C₁₋₆ alkyl, optionally substituted C₃₋₆ cycloalkyl, heterocyclo, optionally substituted heteroaryl, (heterocyclo)alkyl;

R^{7a} is selected from optionally substituted C₁₋₆ alkyl and optionally substituted (heterocyclo)alkyl; and

R^{7b} is selected from hydrogen and C₁₋₄ alkyl.

In one embodiment, the compound for use in in the treatment of non-alcoholic steatohepatitis in a patient in need thereof comprises a compound of formula (I) that is further given by formula (II):

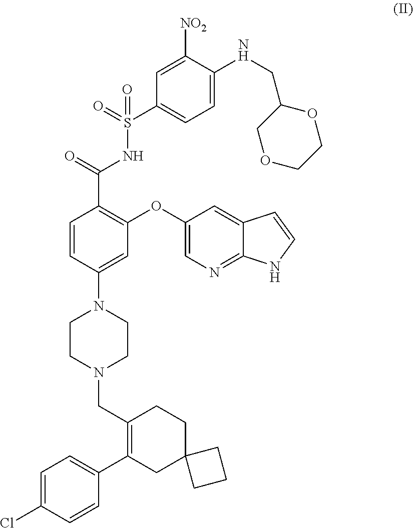 [SMILES: O=C(NS(=O)(=O)c1ccc(NCC2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, hydrate, solvate, stereoisomer, or tautomer thereof.

In one embodiment, the compound for use in in the treatment of non-alcoholic steatohepatitis in a patient in need thereof comprises a compound selected from:

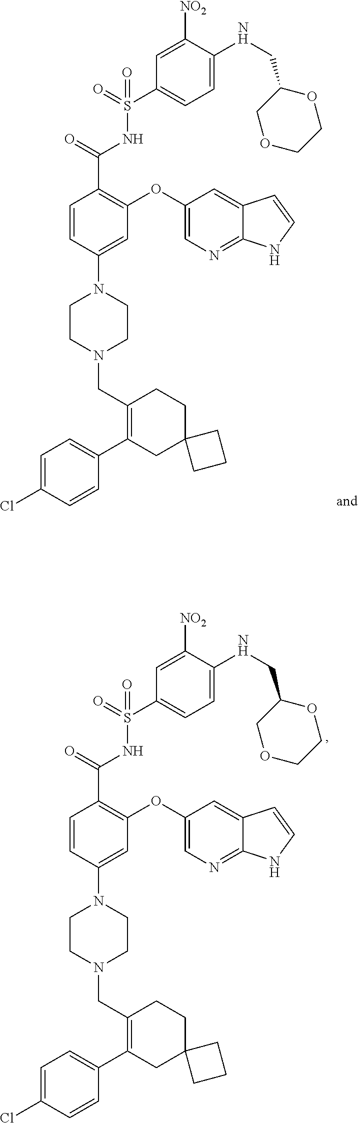 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, solvate, hydrate, or tautomer thereof.

In one embodiment, the compound for use in in the treatment of non-alcoholic steatohepatitis in a patient in need thereof comprises:

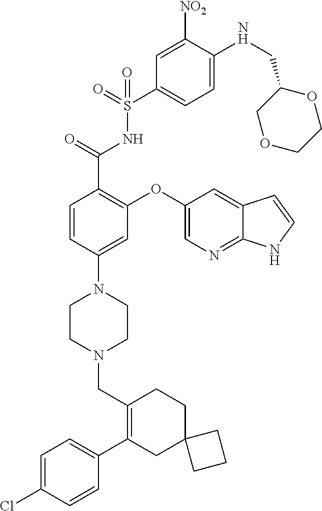 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, hydrate, solvate, or tautomer thereof.

In one embodiment, the compound for use in in the treatment of non-alcoholic steatohepatitis in a patient in need thereof comprises:

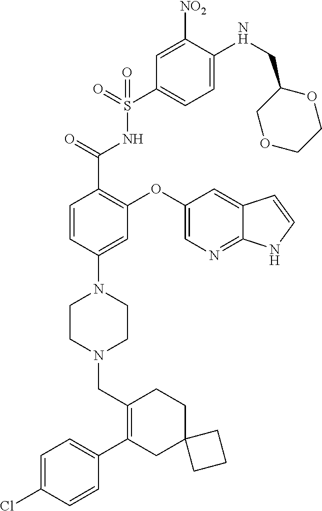 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, hydrate, solvate, or tautomer thereof.

In one embodiment, the compound for use in in the treatment of non-alcoholic steatohepatitis in a patient in need thereof comprises a compound selected from a group consisting of the compounds recited in Table 1, as described herein.

In one embodiment, a compound for use in the treatment of non-alcoholic steatohepatitis in a patient in need thereof comprises (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide.

In one embodiment, the compound is for use in the treatment of non-alcoholic steatohepatitis in a patient, wherein the patient is diagnosed as having one or more diseases selected from cardiovascular disease, chronic kidney disease, type 2 diabetes mellitus, obesity, and metabolic syndrome wherein the metabolic syndrome is selected from hypertension, hyperglycaemia, hyperlipemia, and insulin resistance (IR).

In one embodiment, the compound is for use in the treatment of non-alcoholic steatohepatitis in a patient, wherein the compound or pharmaceutical composition is administered to the patient in need thereof at a dose sufficient to elicit one or more effects selected from the group consisting of: reduced liver steatosis, reduced lobular inflammation, reduced hepatocellular ballooning, and reduced liver fibrosis.

In one embodiment, the compound is for use in the treatment of non-alcoholic steatohepatitis in a patient, wherein the compound or pharmaceutical composition is administered to the patient in need thereof at a dose sufficient to reduce liver steatosis in the patient.

In one embodiment, the compound is for use in the treatment of non-alcoholic steatohepatitis in a patient, wherein the compound or pharmaceutical composition is administered to the patient in need thereof at a dose sufficient to reduce lobular inflammation in the patient.

In one embodiment, the compound is for use in the treatment of non-alcoholic steatohepatitis in a patient, wherein the compound or pharmaceutical composition is administered to the patient in need thereof at a dose sufficient to reduce lobular hepatocellular ballooning in the patient.

In one embodiment, the compound is for use in the treatment of non-alcoholic steatohepatitis in a patient, wherein the compound or pharmaceutical composition is administered to the patient in need thereof at a dose sufficient to reduce liver fibrosis in the patient.

### BRIEF DESCRIPTION OF THE DRAWINGS

FIG. 1A shows HE-stained cross-sections of liver tissue from mice with induced non-alcoholic steatohepatitis treated with Compound 1 and/or OCA in comparison to untreated controls.

FIG. 1B shows NAS score analysis of mice with induced non-alcoholic steatohepatitis treated with Compound 1 and/or OCA in comparison to untreated controls.

FIG. 2A shows Sirius Red-stained cross-sections of liver tissue from mice with induced non-alcoholic steatohepatitis treated with Compound 1 and/or OCA in comparison to untreated controls.

FIG. 2B shows fibrosis score analyses of mice with induced non-alcoholic steatohepatitis treated with Compound 1 and/or OCA in comparison to untreated controls.

FIG. 3A shows HE-stained cross-sections of liver tissue from mice with induced non-alcoholic steatohepatitis treated with Compound 1 or GFT505 in comparison to untreated controls.

FIG. 3B shows NAS score analysis of mice with induced non-alcoholic steatohepatitis treated with Compound 1 or GFT505 in comparison to untreated controls.

FIG. 4A shows Sirius Red-stained cross-sections of liver tissue from mice with induced non-alcoholic steatohepatitis treated with Compound 1 or GFT505 in comparison to untreated controls.

FIG. 4B shows fibrosis score analyses of mice with induced non-alcoholic steatohepatitis treated with Compound 1 or GFT505 in comparison to untreated controls.

### DETAILED DESCRIPTION

The present disclosure relates to compounds, or pharmaceutically acceptable salt, solvates, hydrates, tautomers, and stereoisomers thereof, capable of modulating Bcl-2 family proteins. Compounds capable of modulating Bcl-2 family proteins are useful in treating, preventing, or ameliorating diseases and disorders associated with the activity of Bcl-2 family proteins.

In some aspects, the disclosure features methods of treating or ameliorating non-alcoholic steatohepatitis (NASH) in a patient by administering to the patient in need thereof a therapeutically effective amount of a compound capable of modulating Bcl-2 family proteins, or a pharmaceutical composition comprising a compound capable of modulating Bcl-2 family proteins.

### Definitions

The articles “a” and “an” are used in this disclosure to refer to one or more than one (e.g., to at least one) of the grammatical object of the article. By way of example, “an element” means one element or more than one element.

The term “and/or” is used in this disclosure to mean either “and” or “or” unless indicated otherwise.

The term “about,” as used herein, includes the recited number ±10%. Thus, “about 10” means 9 to 11.

In the present disclosure, the term “alkyl” as used by itself or as part of another group refers to unsubstituted straight- or branched-chain aliphatic hydrocarbons containing one to twelve carbon atoms, i.e., C₁₋₁₂ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. In another embodiment, the alkyl group is a branched chain C_{3_4} alkyl group. In another embodiment, the alkyl group is a straight or branched chain C_{3_4} alkyl group. In another embodiment, the alkyl group is partially or completely deuterated, i.e., one or more hydrogen atoms of the alkyl group are replaced with deuterium atoms. Non-limiting exemplary C₁₋₁₂ alkyl groups include methyl, —CD₃, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tertbutyl, and iso-butyl. Non-limiting exemplary C₁₋₄ groups include methyl, ethyl, propyl, isopropyl, and tert-butyl.

In the present disclosure, the term “optionally substituted alkyl” as used by itself or as part of another group refers to an alkyl that is unsubstituted or substituted with one, two, or three substituents independently selected from the group consisting of halo, nitre, cyano, hydroxy, alkoxy, amino, alkylamino, dialkylamino, and optionally substituted aryl. In one embodiment, the optionally substituted alkyl is substituted with two substituents. In another embodiment, the optionally substituted alkyl is substituted with one substituent. In another embodiment, the optionally substituted alkyl is unsubstituted. Non-limiting exemplary optionally substituted alkyl groups include —CH₂Ph, —CH₂CH₂NO₂, —CH₂CH₂OH, —CH₂CH₂OCH₃, and —CH₂CH₂F.

In the present disclosure, the term “cycloalkyl” as used by itself or as part of another group refers to unsubstituted saturated or partially unsaturated, e.g., containing one or two double bonds, cyclic aliphatic hydrocarbons containing one to three rings having from three to twelve carbon atoms, i.e., C₃₋₁₂ cycloalkyl, or the number of carbons designated. In one embodiment, the cycloalkyl group has two rings. In one embodiment, the cycloalkyl group has one ring. In another embodiment, the cycloalkyl group is a C₃₋₈ cycloalkyl. In another embodiment, the cycloalkyl group is a C₃₋₆ cycloalkyl. In another embodiment, the cycloalkyl group is a C₃₋₅ cycloalkyl. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, cyclopentenyl, cyclopentanone, spiro[3.3]heptane, and bicyclo[3.3.1]nonane.

In the present disclosure, the term “optionally substituted cycloalkyl” as used by itself or as part of another group refers to a cycloalkyl that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo. In one embodiment, the optionally substituted cycloalkyl is substituted with two substituents. In another embodiment, the optionally substituted cycloalkyl is substituted with one substituent. In another embodiment, the optionally substituted cycloalkyl is unsubstituted.

In the present disclosure, the term “haloalkyl” as used by itself or as part of another group refers to an alkyl substituted by one or more fluorine, chlorine, bromine and/or iodine atoms. In one embodiment, the alkyl group is substituted by one, two, or three fluorine and/or chlorine atoms. In another embodiment, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1, 1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “alkoxy” as used by itself or as part of another group refers to an optionally substituted alkyl attached to a terminal oxygen atom. In one embodiment, the alkoxy group is a C₁₋₆ alkyl attached to a terminal oxygen atom. In another embodiment, the alkoxy group is a C₁₋₄ alkyl attached to a terminal oxygen atom. Nonlimiting exemplary alkoxy groups include methoxy, ethoxy, and tert-butoxy.

In the present disclosure, the term “heterocyclo” as used by itself or as part of another group refers to unsubstituted saturated and partially unsaturated, e.g., containing one or two double bonds, cyclic groups containing one, two, or three rings having from three to fourteen ring members, i.e., a 3- to 14-membered heterocyclo, wherein at least one carbon atom of one of the rings is replaced with a heteroatom. The term “heterocyclo” is meant to include cyclic ureido groups such as imidazolidinyl-2-one, cyclic amide groups such as β-lactam, γ-lactam, δ-lactam and ε-lactam, and cyclic carbamate groups such as oxazolidinyl-2-one. In one embodiment, the heterocyclo group is a 4-, 5-, 6-, 7- or 8-membered cyclic group containing one ring and one or two oxygen and/or nitrogen atoms. In one embodiment, the heterocyclo group is a 5- or 6-membered cyclic group containing one ring and one or two nitrogen atoms. In one embodiment, the heterocyclo group is an 8-, 9-, 10-, 11-, or 12-membered cyclic group containing two rings and one or two nitrogen atoms. In one embodiment, the heterocyclo group is a 4- or 5-membered cyclic group containing one ring and one oxygen atom. The heterocyclo can be optionally linked to the rest of the molecule through a carbon or nitrogen atom. Non-limiting exemplary heterocyclo groups include 1,4-dioxane, 2-oxopyrrolidin-3-yl, 2-imidazolidinone, piperidinyl, morpholinyl, piperazinyl, pyrrolidinyl, 8-azabicyclo[3.2.1]octane (nortropane), 6-azaspiro[2.5]octane, 6-azaspiro[3.4]octane, indolinyl, indolinyl-2-one, and 1,3-dihydro-2H-benzo[d]imidazol-2-one.

In the present disclosure, the term “optionally substituted heterocyclo” as used herein by itself or part of another group refers to a heterocyclo that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo.

In the present disclosure, the term “(heterocyclo)alkyl” as used by itself or as part of another group refers to an alkyl substituted with one optionally substituted heterocyclo group. In one embodiment, the (heterocyclo)alkyl is a C₁₋₄ alkyl substituted with one optionally substituted 4- to 6-membered heterocyclo group. The heterocyclo can be linked to the alkyl group through a carbon or nitrogen atom. Non-limiting exemplary (heterocyclo)alkyl groups include:

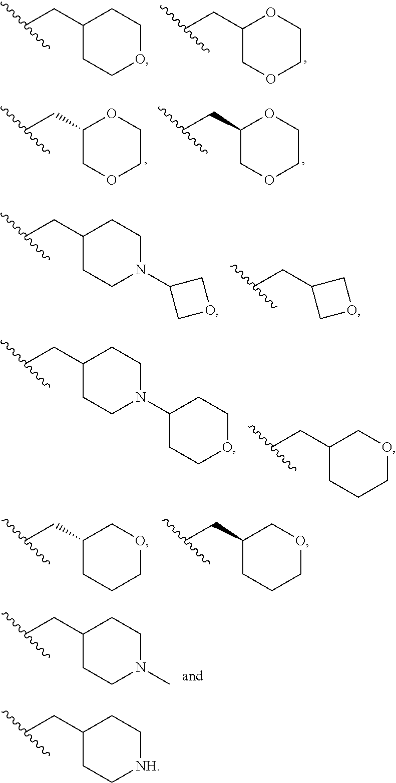

As used herein, the term “heteroaryl” refers to a monocyclic aromatic radical of 5 to 14 ring atoms, containing one or more ring heteroatoms selected from the group consisting of N, O, and S, the remaining ring atoms being C. Examples include, but are not limited to, furyl, thienyl, pyrrolyl, pyridinyl, pyrazolyl, pyrimidinyl, imidazolyl, isoxazolyl, oxazolyl, oxadiazolyl, pyrazinyl, thiophen-2-yl, isothiazolyl, thiazolyl, thiadiazolyl, triazolyl, triazinyl.

As used herein, the term “optionally substituted heteroaryl” refers to a heteroaryl that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo.

As used herein, the term “isomer” refers to compounds that have the same composition and molecular weight but differ in physical and/or chemical properties. The structural difference may be in constitution (e.g., geometric isomers) or in the ability to rotate a plane of polarized light (stereoisomers). With regard to stereoisomers, the compounds of the disclosure may have one or more asymmetric carbon atoms and may occur as racemates, racemic mixtures or as individual enantiomers or diastereomers.

As used herein, the term “stereoisomers” or “stereoisomeric forms” are general terms for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R-S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The terms “enantiomerically pure” or “enantiopure” refer to a sample of a chiral substance all of whose molecules (within the limits of detection) have the same chirality sense. In one embodiment, Compounds of the Disclosure having one or more chiral centers are enantiopure.

The terms “enantiomerically enriched” or “enantioenriched” refer to a sample of a chiral substance whose enantiomeric excess is greater than 50%, e.g., about 60% or more, about 70% or more, about 80% or more, about 90% or more, about 95% or more, about 98% or more, or about 99% or more. Enantiomerically enriched compounds may be enantiomerically pure.

The term “pharmaceutically acceptable salt” as used herein, refers to any salt, e.g., obtained by reaction with an acid or a base, of a compound of the disclosure that is physiologically tolerated in the target patient (e.g., a mammal, e.g., a human).

The use of the terms “salt” and the like, is intended to equally apply to the salt of enantiomers, stereoisomers, rotamers, tautomers, positional isomers, and racemates of the inventive compounds.

The term “solvate” as used herein is a combination, physical association and/or solvation of a compound of the present disclosure with a solvent molecule such as, e.g., a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates.

A “patient” or “subject” is a mammal, e.g., a human, mouse, rat, guinea pig, dog, cat, horse, cow, pig, or non-human primate, such as a monkey, chimpanzee, baboon, or rhesus.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms thereof contemplate administering a therapeutically effective amount of a compound of the disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

The term “administer”, “administering”, or “administration” as used in this disclosure refers to either directly administering a disclosed compound, a pharmaceutically acceptable salt of a disclosed compound or a composition to a subject, a pharmaceutically acceptable salt of a compound, or a composition to a subject, which can form an equivalent amount of active compound within the subject's body.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient that is sufficient, when administered by a method of the disclosure, to efficaciously deliver the active ingredient(s) for the treatment of condition or disease of interest to an individual in need thereof.

The term “carrier”, as used in this disclosure, encompasses carriers, excipients, and diluents and means a material, composition or vehicle, such as a liquid or solid filler, diluent, excipient, solvent, or encapsulating material, involved in carrying or transporting a pharmaceutical agent from one organ, or portion of the body, to another organ, or portion of the body of a subject.

In the present disclosure, the term “Bcl-2 proteins” or “Bcl-2 family of proteins” refers to any one or more of the following proteins: Bax, Bak, Bid, Bcl-2, Bcl-xL, Mcl-1, Bcl-w, Bfl-1/Al, Bim, Puma, Bad, Bik/Blk, Noxa, Bmf, Hrk/DP5, and Beclin-1. See Cold Spring Harb Perspect Biol 2013; 5:a008714.

The term “disease” or “condition” or “disorder” denotes disturbances and/or anomalies that as a rule are regarded as being pathological conditions or functions, and that can manifest themselves in the form of particular signs, symptoms, and/or malfunctions. Compounds of the disclosure inhibit Bcl-2 proteins, such as Bcl-2 and/or Bcl-xL, and can be used in treating or preventing diseases, conditions, or disorders such as non-alcoholic steatohepatitis.

In some embodiments, the compounds of the disclosure can be used to treat a “Bcl-2 protein mediated disorder,” e.g., a Bcl-2-mediated disorder and/or a Bcl-xL-mediated disorder. A Bcl-2 protein mediated disorder is any pathological condition in which a Bcl-2 protein is known to play a role.

### Compounds of the Disclosure

In one aspect, the disclosure relates to compounds of formula (I):

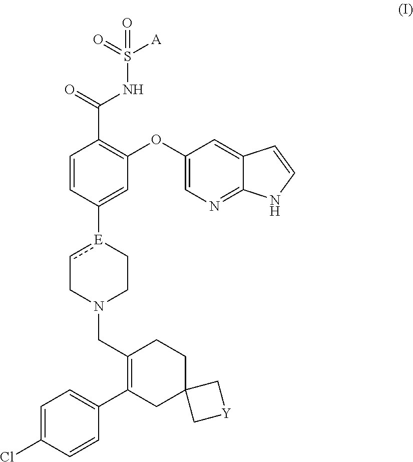

or pharmaceutically acceptable salts, hydrates, solvates, stereoisomers, or tautomers thereof, wherein:

A is selected from the group consisting of:

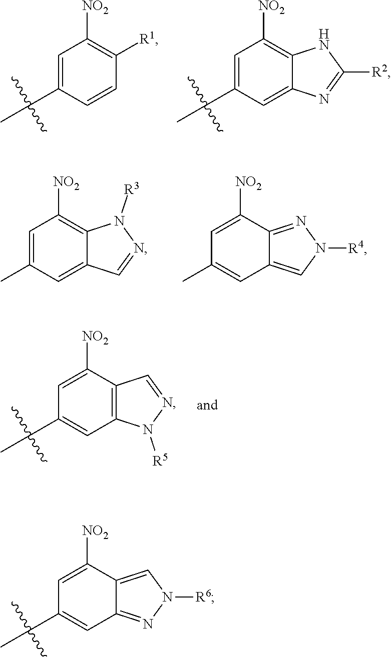

E is selected from the group consisting of:
a carbon atom, wherein [symbol] is a double bond;
—C(H)—, wherein [symbol] is a single bond; and
a nitrogen atom, wherein [symbol] is a single bond;

Y is selected from —C(H)— and —O—;

R¹ is selected from hydrogen and —N(R^{7a})(R^{7b});

R², R³, R⁴, R⁵, and R⁶ are each independently selected from the group consisting of hydrogen, optionally substituted C₁₋₆ alkyl, optionally substituted C₃₋₆ cycloalkyl, heterocyclo, optionally substituted heteroaryl, (heterocyclo)alkyl;

R^{7a} is selected from optionally substituted C₁₋₆ alkyl and optionally substituted (heterocyclo)alkyl; and

R^{7b} is selected from hydrogen and C₁₋₄ alkyl.

In some embodiments, A is

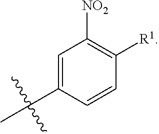

In some embodiments, A is

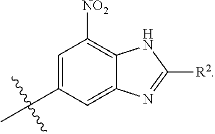

In some embodiments, A is

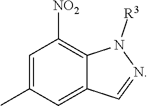

In some embodiments, A is

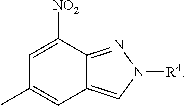

In some embodiments, A is

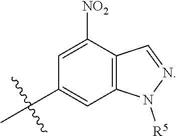

In some embodiments, A is

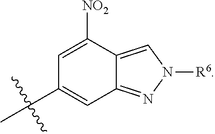

In some embodiments, E is a carbon atom, wherein [symbol] is a double bond. In some embodiments, E is —C(H)—, wherein [symbol] is a single bond. In some embodiments, E is a nitrogen atom, wherein [symbol] is a single bond.

In some embodiments, R¹ is hydrogen. In some embodiments, R¹ is —N(R^{7a})(R^{7b}).

In some embodiments, R² is an optionally substituted C₁₋₆ alkyl. In some embodiments, R² is an optionally substituted C₁₋₄ alkyl. In some embodiments, R² is an optionally substituted C₃ alkyl. In some embodiments, R² is isopropyl. In some embodiments, R² is an optionally substituted C₃₋₆ cycloalkyl. In some embodiments, R² is an optionally substituted C₃₋₅ cycloalkyl. In some embodiments, R² is cyclopropyl. In some embodiments, R² is an optionally substituted heteroaryl. In some embodiments, R² is pyridinyl.

In some embodiments, R³ is a (heterocyclo)alkyl. In some embodiments, R³ is

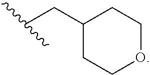 [SMILES: *CC1CCOCC1]

In some embodiments, R⁴ is a (heterocyclo)alkyl. In some embodiments, R⁴ is

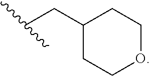 [SMILES: *CC1CCOCC1]

In some embodiments, R⁵ is a (heterocyclo)alkyl. In some embodiments, R⁵ is

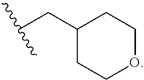 [SMILES: *CC1CCOCC1]

In some embodiments, R⁶ is a heterocyclo. In some embodiments, R⁶ is tetrahydro-2H-pyranyl.

In some embodiments, R⁶ is a (heterocyclo)alkyl. In some embodiments, R⁶ is

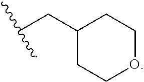 [SMILES: *CC1CCOCC1]

In some embodiments, R⁶ is a heterocyclo. In some embodiments, R⁶ is tetrahydro-2H-pyranyl.

In some embodiments, R^{7a} is an optionally substituted C₁₋₆ alkyl. In some embodiments, R^{7a} is an optionally substituted C₁₋₄ alkyl. In some embodiments, R^{7a} is an optionally substituted methyl. In some embodiments, R^{7a} is methyl. In some embodiments, R^{7a} is an optionally substituted (heterocyclo)alkyl. In some embodiments, R^{7a} is

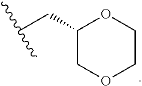 [SMILES: *C[C@H]1COCCO1]

In some embodiments, R^{7a} is

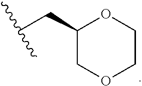 [SMILES: *C[C@@H]1COCCO1]

In some embodiments, R^{7a} is

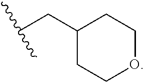 [SMILES: *CC1CCOCC1]

In some embodiments, R^{7b} is hydrogen. In some embodiments, R^{7b} is C₁₋₄ alkyl. In some embodiments, R^{7b} is C₁₋₃ alkyl. In some embodiments, R^{7b} is methyl.

In some embodiments, the disclosure relates to a compound of formula (I), selected from Table 1, or a pharmaceutically acceptable salt, hydrate, solvate, stereoisomer, or tautomer thereof.

TABLE 1

| Cpd. | | |
|---|---|---|
| No. | Structure | Name |
| 1 | 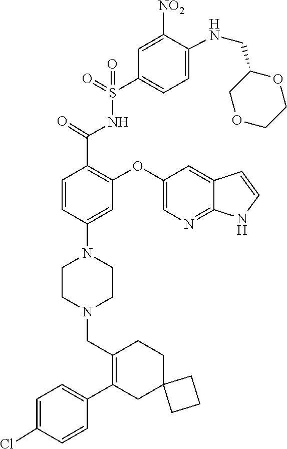 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)benzamide |
| 2 | 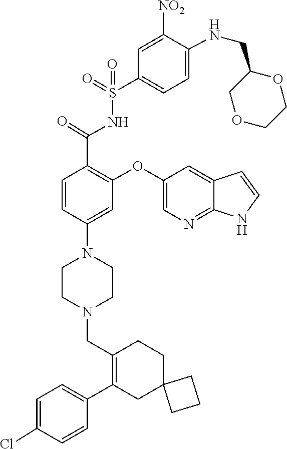 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (R)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4- chlorophenyl)spiro[3.5]non-6-en-7- yl)methyl)piperazin-1-yl)benzamide |
| 3 | 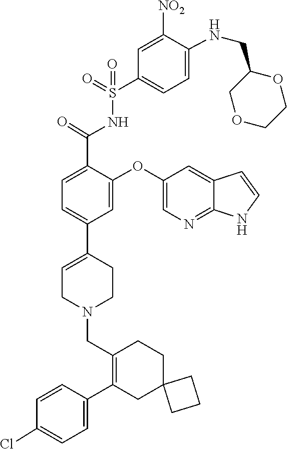 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CCC4)CC3)CC2)cc1Oc1cnc2[nH]ccc2c1] | (R)-N-((4-(((1,4-dioxan-2-yl)methyl)amino)-3- nitrophenyl)sulfonyl)-2-((1H-pyrrolo[2,3- b]pyridin-5-yl)oxy)-4-(1-((6-(4- chlorophenyl)spiro[3.5]non-6-en-7-yl)methyl)- 1,2,3,6-tetrahydropyridin-4-yl)benzamide |
| 4 | 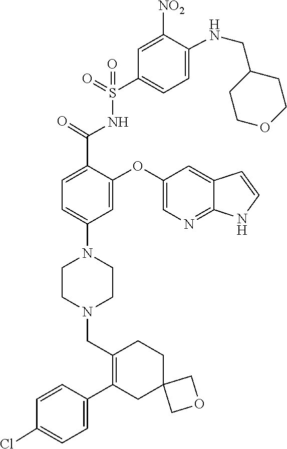 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)-4-(4-((6- (4-chlorophenyl)-2-oxaspiro[3.5]non-6-en-7- yl)methyl)piperazin-1-yl)-N-((3-nitro-4- (((tetrahydro-2H-pyran-4- yl)methyl)amino)phenyl)sulfonyl)benzamide |
| 5 | 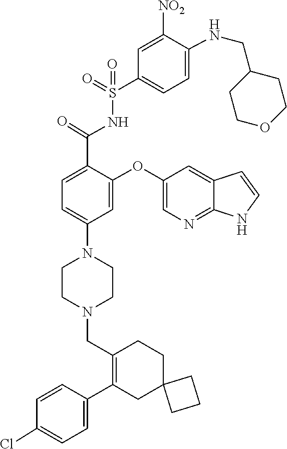 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((3-nitro-4- (((tetrahydro-2H-pyran-4- yl)methyl)amino)phenyl)sulfonyl)benzamide |
| 6 | 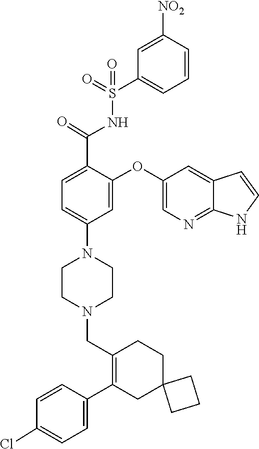 [SMILES: O=C(NS(=O)(=O)c1cccc([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((3- nitrophenyl)sulfonyl)benzamide |
| 7 | 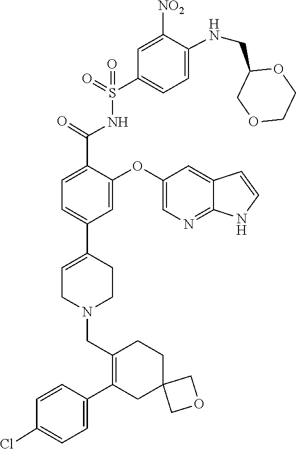 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CC3)COC4)CC2)cc1Oc1cnc2[nH]ccc2c1] | (R)-N-((4-(((1,4-dioxan-2-yl)methyl)amino)-3- nitrophenyl)sulfonyl)-2-((1H-pyrrolo[2,3- b]pyridin-5-yl)oxy)-4-(1-((6-(4-chlorophenyl)-2- oxaspiro[3.5]non-6-en-7-yl)methyl)-1,2,3,6- tetrahydropyridin-4-yl)benzamide |
| 8 | 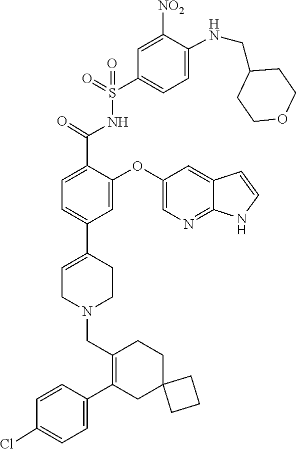 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(1-((6- (4-chlorophenyl)spiro[3.5]non-6-en-7-yl)methyl)- 1,2,3,6-tetrahydropyridin-4-yl)-N-((3-nitro-4- (((tetrahydro-2H-pyran-4- yl)methyl)amino)phenyl)sulfonyl)benzamide |
| 9 | 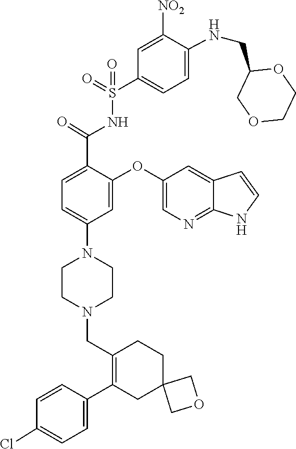 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CC3)COC4)CC2)cc1Oc1cnc2[nH]ccc2c1] | (R)-N-((4-(((1,4-dioxan-2-yl)methyl)amino)-3- nitrophenyl)sulfonyl)-2-((1H-pyrrolo[2,3- b]pyridin-5-yl)oxy)-4-(4-((6-(4-chlorophenyl)-2- oxaspiro[3.5]non-6-en-7-yl)methyl)piperazin-1- yl)benzamide |
| 10 | 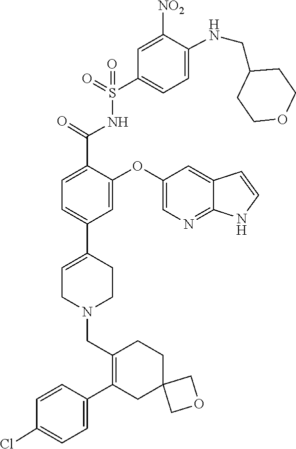 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)-4-(1-((6- (4-chlorophenyl)-2-oxaspiro[3.5]non-6-en-7- yl)methyl)-1,2,3,6-tetrahydropyridin-4-yl)-N-((3- nitro-4-(((tetrahydro-2H-pyran-4- yl)methyl)amino)phenyl)sulfonyl)benzamide |
| 11 | 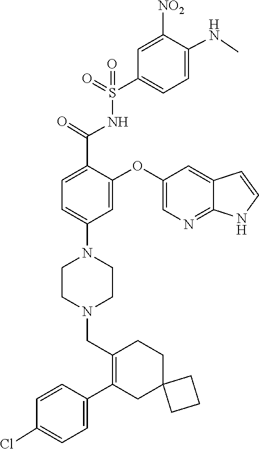 [SMILES: CNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((4-(methylamino)-3- nitrophenyl)sulfonyl)benzamide |
| 12 | 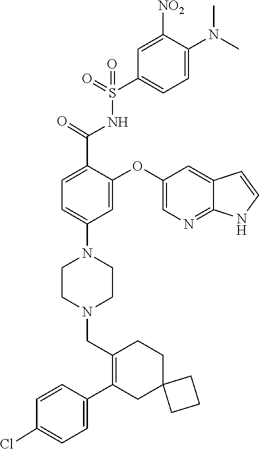 [SMILES: CN(C)c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((4-(dimethylamino)- 3-nitrophenyl)sulfonyl)benzamide |
| 13 | 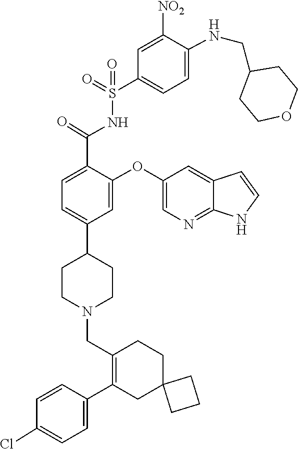 [SMILES: O=C(NS(=O)(=O)c1ccc(NCC2CCOCC2)c([N+](=O)[O-])c1)c1ccc(C2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(1-((6- (4-chlorophenyl)spiro[3.5]non-6-en-7- yl)methyl)piperidin-4-yl)-N-((3-nitro-4- (((tetrahydro-2H-pyran-4- yl)methyl)amino)phenyl)sulfonyl)benzamide |
| 14 | 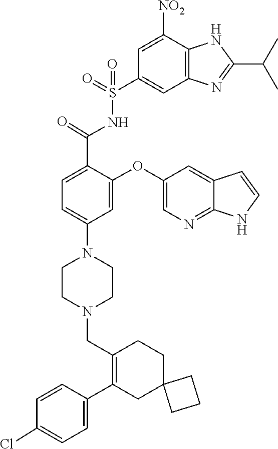 [SMILES: CC(C)c1nc2cc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc([N+](=O)[O-])c2[nH]1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((2-isopropyl-7-nitro- 1H-benzo[d]imidazol-5-yl)sulfonyl)benzamide |
| 15 | 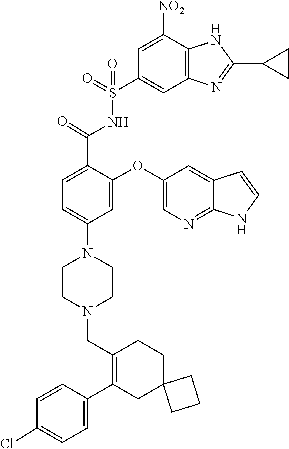 [SMILES: O=C(NS(=O)(=O)c1cc([N+](=O)[O-])c2[nH]c(C3CC3)nc2c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((2-cyclopropyl-7- nitro-1H-benzo[d]imidazol-5- yl)sulfonyl)benzamide |
| 16 | 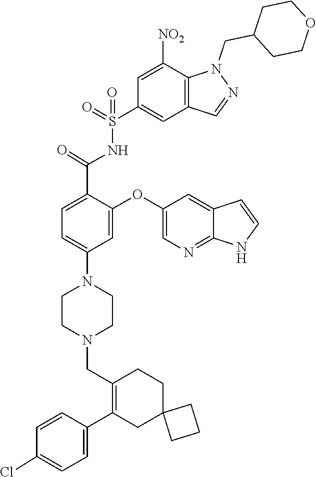 [SMILES: O=C(NS(=O)(=O)c1cc([N+](=O)[O-])c2c(cnn2CC2CCOCC2)c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((7-nitro-1- ((tetrahydro-2H-pyran-4-yl)methyl)-1H-indazol-5- yl)sulfonyl)benzamide |
| 17 | 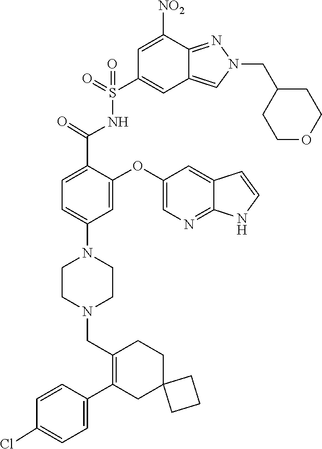 [SMILES: O=C(NS(=O)(=O)c1cc([N+](=O)[O-])c2nn(CC3CCOCC3)cc2c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((7-nitro-2- ((tetrahydro-2H-pyran-4-yl)methyl)-2H-indazol-5- yl)sulfonyl)benzamide |
| 18 | 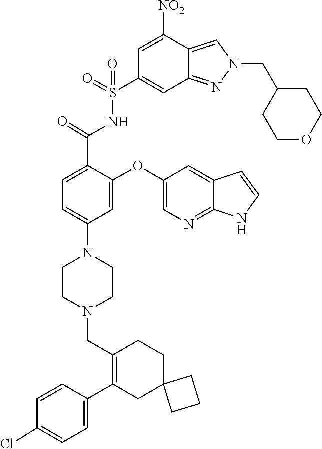 [SMILES: O=C(NS(=O)(=O)c1cc([N+](=O)[O-])c2cn(CC3CCOCC3)nc2c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((4-nitro-2- ((tetrahydro-2H-pyran-4-yl)methyl)-2H-indazol-6- yl)sulfonyl)benzamide |
| 19 | 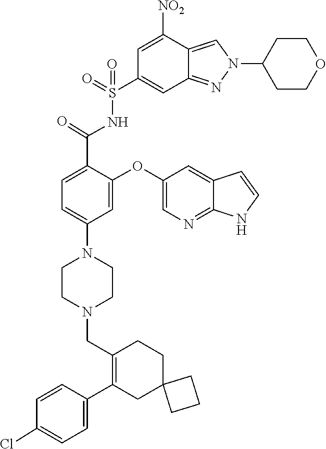 [SMILES: O=C(NS(=O)(=O)c1cc([N+](=O)[O-])c2cn(C3CCOCC3)nc2c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((4-nitro-2- (tetrahydro-2H-pyran-4-yl)-2H-indazol-6- yl)sulfonyl)benzamide |
| 20 | 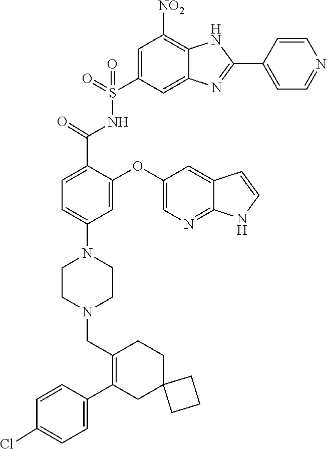 [SMILES: O=C(NS(=O)(=O)c1cc([N+](=O)[O-])c2[nH]c(-c3ccncc3)nc2c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)-4-(4-((6- (4-chlorophenyl)spiro[3.5]non-6-en-7- yl)methyl)piperazin-1-yl)-N-((7-nitro-2-(pyridin-4- yl)-1H-benzo[d]imidazol-5-yl)sulfonyl)benzamide |

Compounds of formula (I) may contain one or more asymmetric centers and may thus give rise to enantiomers, diastereomers, and other stereoisomeric forms. The present disclosure is meant to encompass the use of all such possible forms including racemic and resolved forms, and mixtures thereof. The individual stereoisomers, e.g., enantiomers, can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also intended to be encompassed by the present disclosure. The assay results may reflect the data collected for the racemic form, the enantiomerically pure form, or any other form in terms of stereochemistry. Individual stereoisomers of the compounds of the disclosure may be, for example, substantially free of other isomers, or may be admixed, for example, as racemates or with all other, or other selected, stereoisomers.

In some embodiments, compounds of formula (I) having one or more chiral centers are enantioenriched.

In addition, the present disclosure embraces all geometric and positional isomers. For example, if a compound of formulas (I) incorporates a double bond or a fused ring, both the cis- and trans-forms, as well as mixtures, are embraced within the scope of the disclosure. If the compound contains a double bond, the substituent may be in the E or Z configuration, unless otherwise indicated. If the compound contains a disubstituted cycloalkyl, the cycloalkyl substituent may have a cis- or trans configuration, unless otherwise indicated.

In some embodiments, the disclosure relates to compounds of formula (I) that are further given by formula (II):

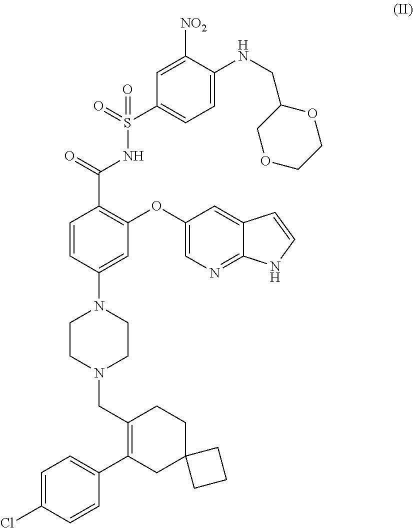 [SMILES: O=C(NS(=O)(=O)c1ccc(NCC2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or pharmaceutically acceptable salts, hydrates, solvates, stereoisomers, or tautomers thereof.

In some embodiments, the disclosure relates to a compound of formula (II) selected from the group consisting of Compound (1) and Compound (2):

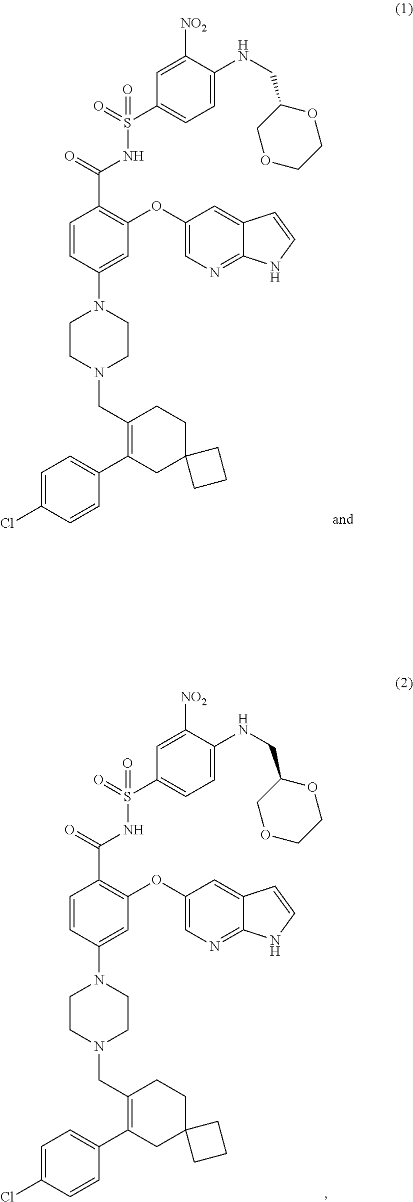 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, hydrate, solvate, or tautomer thereof.

In some embodiments, the disclosure relates to Compound (1)

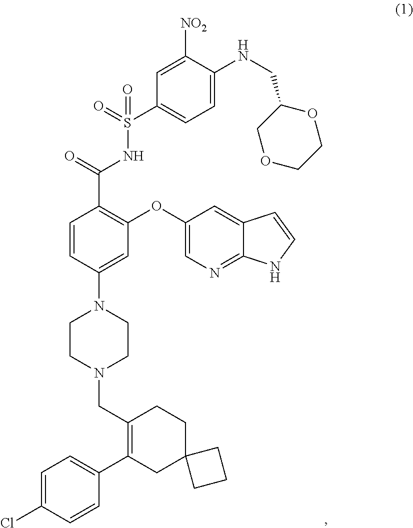 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, hydrate, solvate, or tautomer thereof.

In some embodiments, the disclosure relates to Compound (2)

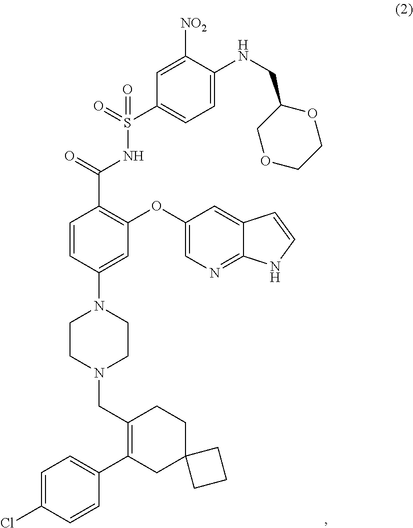 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt, hydrate, solvate, or tautomer thereof.

In some embodiments, the disclosure relates to a mixture of Compound (1) and Compound (2).

In some embodiments, the disclosure relates to a compound selected from:
(S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide; and
(R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide,
   or a pharmaceutically acceptable salt, hydrate, solvate, or tautomer thereof.

In some embodiments, the disclosure relates to (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, or a pharmaceutically acceptable salt, hydrate, solvate, or tautomer thereof.

In some embodiments, the disclosure relates to (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, or a pharmaceutically acceptable salt, hydrate, solvate, or tautomer thereof.

In some embodiments, the disclosure relates to a mixture of (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide and (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, or pharmaceutically acceptable salts, hydrates, solvates, or tautomers thereof.

The present disclosure encompasses any of the compounds of formula (I) being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, sulfur, and chlorine, such as 2H (or deuterium (D)), ³H, ¹¹C, ¹³C, ¹⁴C, ¹⁵N, ¹⁸O, ¹⁷O, ³⁵S, and ³⁶Cl, respectively, e.g., ³H, ¹¹C, and ¹⁴C. In one embodiment, provided is a composition wherein substantially all of the atoms at a position within the compound of formula (I) are replaced by an atom having a different atomic mass or mass number. In another embodiment, provided is a composition wherein a portion of the atoms at a position within the compound of formula (I) are replaced, i.e., the compound of formula (I) is enriched at a position with an atom having a different atomic mass or mass number. Isotopically-labelled compounds of formula (I) can be prepared by methods known in the art.

The present disclosure encompasses the preparation and use of salts of compounds of formula (I), including non-toxic pharmaceutically acceptable salts. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

When compounds of the present invention contain relatively acidic functionalities, base addition salts can be obtained by contacting the neutral form of such compounds with a sufficient amount of the desired base, either neat or in a suitable inert solvent. Salts derived from pharmaceutically-acceptable inorganic bases include aluminum, ammonium, calcium, copper, iron(III), iron(II), lithium, magnesium, manganese, potassium, sodium, zinc, and the like. Salts derived from pharmaceutically-acceptable organic bases include salts of primary, secondary, tertiary and quaternary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When compounds of the present invention contain relatively basic functionalities, acid addition salts can be obtained by contacting the neutral form of such compounds with a sufficient amount of the desired acid, either neat or in a suitable inert solvent. Acids suitable for the preparation of pharmaceutically acceptable acid addition salts include acetic, ascorbic, benzenesulfonic, benzoic, camphorsulfonic, citric, ethanesulfonic, fumaric, gluconic, glucoronic, glutamic, hippuric, hydrobromic, hydrochloric, isethionic, lactic, lactobionic, maleic, malic, mandelic, methanesulfonic, mucic, naphthalenesulfonic, nicotinic, nitric, pamoic, pantothenic, phosphoric, succinic, sulfuric, tartaric, p-toluenesulfonic, and the like.

The compounds of the disclosure may form acid addition salts or base addition salts, which may be pharmaceutically acceptable salts.

The present disclosure encompasses the preparation and use of solvates of compounds of formula (I). Solvates typically do not significantly alter the physiological activity or toxicity of the compounds, and as such may function as pharmacological equivalents. Compounds of formula (I) can be present as solvated forms with a pharmaceutically acceptable solvent, such as water, methanol, ethanol, and the like, and it is intended that the disclosure includes both solvated and unsolvated forms of compounds of formula (I).

In some embodiments, the solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5 (1): Article 12 (2004), and A. L. Bingham et al., Chem. Commun. 603-604 (2001). A typical, non-limiting, process of preparing a solvate would involve dissolving a compound of formula (I) in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

Compounds of formula (I) are capable of modulating the activity of Bcl-2 family proteins. In some embodiments, compounds of formula (I) are capable of modulating the activity of Bcl-2 and/or Bcl-xL. In some embodiments, compounds of formula (I) are capable of inhibiting Bcl-2 and/or Bcl-xL.

Compound (1), (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, is capable of modulating the activity of Bcl-2 family proteins. In some embodiments, Compound (1) is capable of modulating the activity of Bcl-2 and/or Bcl-xL. In some embodiments, Compound (1) is capable of inhibiting Bcl-2 and/or Bcl-xL.

Compound (2), (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, is capable of modulating the activity of Bcl-2 family proteins. In some embodiments, Compound (2) is capable of modulating the activity of Bcl-2 and/or Bcl-xL. In some embodiments, Compound (2) is capable of inhibiting Bcl-2 and/or Bcl-xL.

The present disclosure includes the discovery that compounds of formula (I), and pharmaceutically acceptable salts, solvates, hydrates, stereoisomers, and tautomers thereof, or pharmaceutical compositions comprising a compound of formula (I), are useful for the treatment of diseases or disorders associated with the activity of Bcl-2 family proteins, such non-alcoholic steatohepatitis.

The present disclosure includes the discovery that Compound (1), (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, and pharmaceutically acceptable salts, solvates, hydrates, stereoisomers, and tautomers thereof, or pharmaceutical compositions comprising Compound (1), are useful for the treatment of diseases or disorders associated with the activity of Bcl-2 family proteins, such as non-alcoholic steatohepatitis.

The present disclosure includes the discovery that Compound (2), (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide, and pharmaceutically acceptable salts, solvates, hydrates, stereoisomers, and tautomers thereof, or pharmaceutical compositions comprising Compound (2), are useful for the treatment of diseases or disorders associated with the activity of Bcl-2 family proteins, such as non-alcoholic steatohepatitis.

### Methods of Preparing the Compounds of the Disclosure

The compounds of the present disclosure may be made by a variety of methods, including standard chemistry. Suitable synthetic routes are depicted in the examples given below.

The compounds of the present disclosure, i.e., compounds of formula (I), or a pharmaceutically acceptable salt, hydrate, solvate, stereoisomer, or tautomer thereof, may be prepared by methods known in the art of organic synthesis as set forth in part by the synthetic schemes depicted in the examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of formula (I).

Those skilled in the art will recognize stereocenters exist in the compounds of formula (I). Accordingly, the present disclosure includes both possible stereoisomers (unless otherwise indicated and/or specified in the synthesis) and includes not only racemic compounds but the individual enantiomers and/or diastereomers as well. Unless otherwise indicated, when a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

Compounds of the disclosure are prepared using methods known to those skilled in the art in view of this disclosure, or by the illustrative methods shown in the General Schemes below. For example, methods of preparing the compounds of the disclosure are disclosed in U.S. Pat. No. 10,221,174, the contents of which are incorporated herein in their entirety.

In General Schemes 1-4, presented below, Y, R², and R^{4a} are defined as follows:
Y═CH₂ or O
R²═—NO₂
R^{4a}=A, as defined above with respect to formula (I).

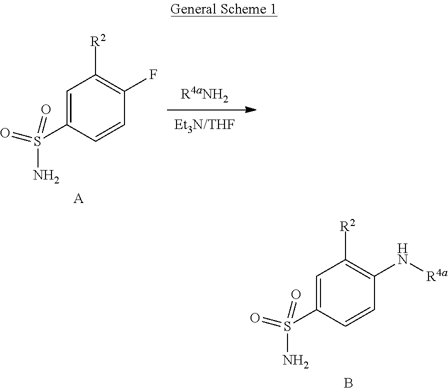

In General Scheme 1, Compound A is reacted with R^{4a}NH₂ in the presence of a base, e.g., triethylamine, to give Compound B.

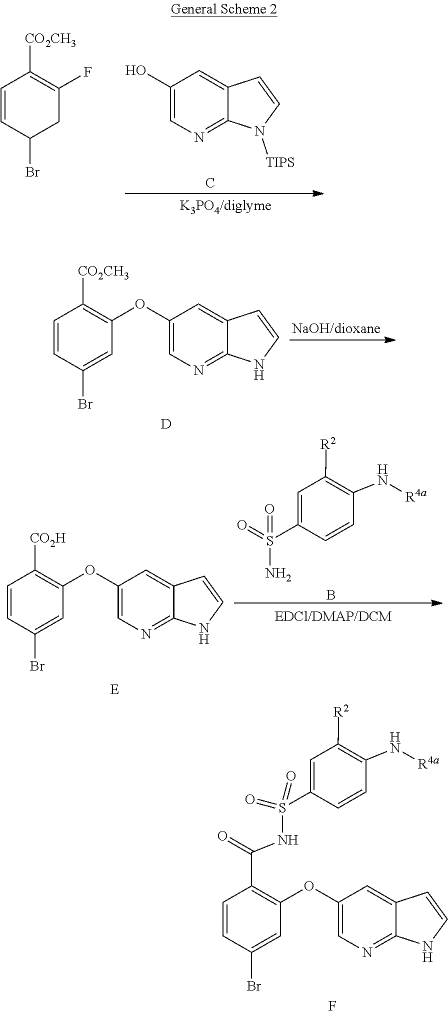

In General Scheme 2, methyl 4-bromo-2-fluorobenzoate is reacted with Compound C to give Compound D, and the ester of Compound D is hydrolyzed to give Compound E. Compound E is coupled with Compound B from General Scheme 1 to give Compound F.

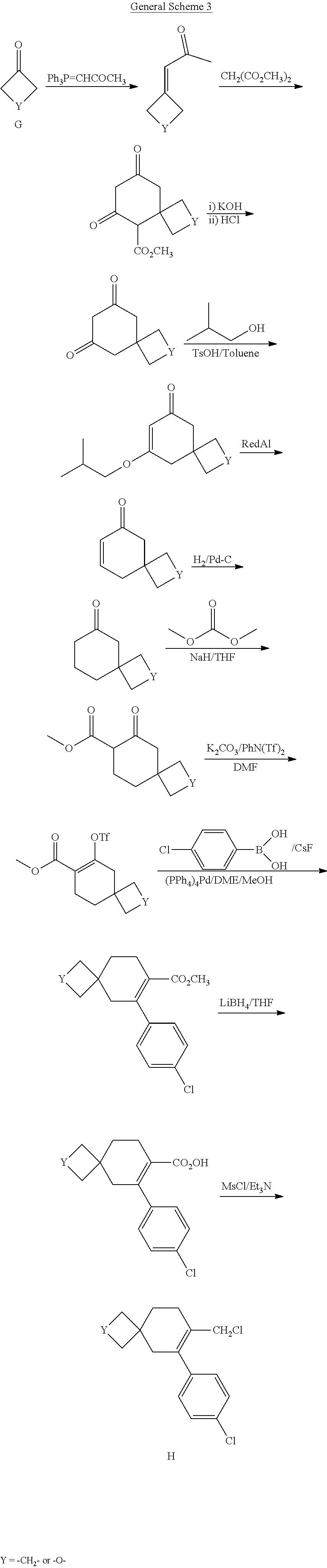

In General Scheme 3, Compound G is transformed Compound H.

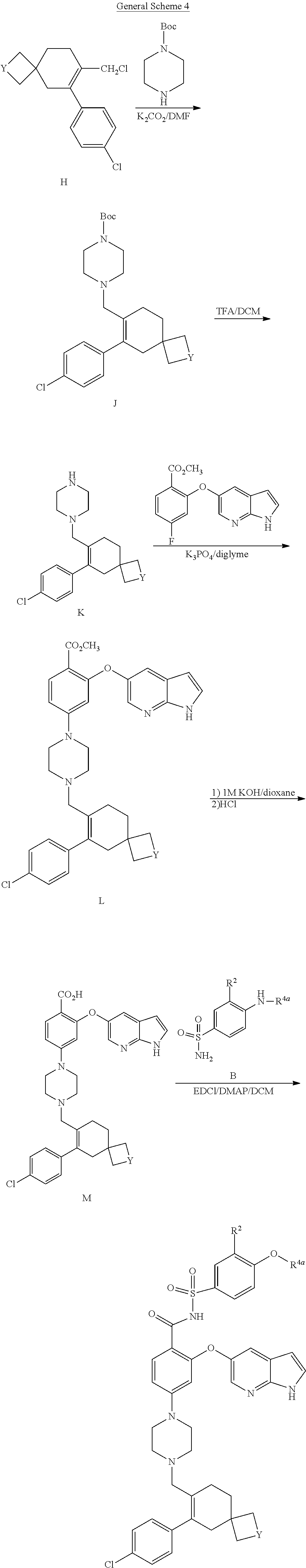

In General Scheme 4, Compound H from General Scheme 3 is reacted with Boc-protected piperidine to give Compound J, and the Boc group is removed to give Compound K. Compound K is reacted with methyl 2-((1H-pyrrolo[2,3-b]pyridin-5-yl)oxy)-4-fluorobenzoate to give Compound L, and the ester of Compound L is hydrolyzed to give Compound M. Compound M is coupled with Compound B from General Scheme 1 to give a compound of formula (I), wherein E is a nitrogen atom and wherein [symbol] is a single bond.

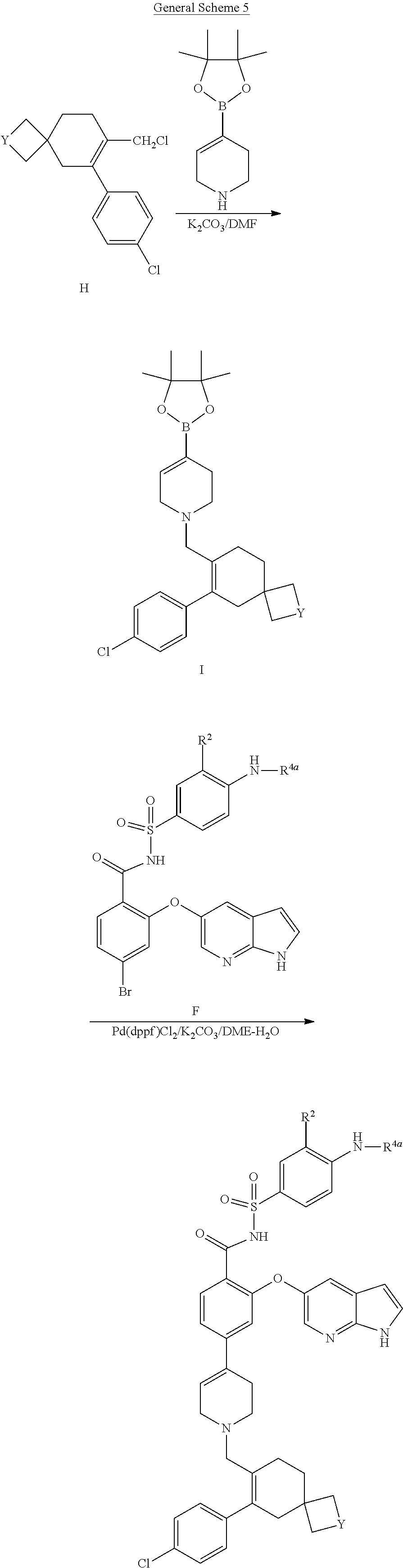

In General Scheme 5, Compound H from General Scheme 3 is reacted with 4-(4,4,5,5-tetramethyl-1,3,2-dioxaborolan-2-yl)-1,2,3,6-tetrahydropyridine to give Compound I. Compound I is coupled with Compound F from General Scheme 2 to give a compound of formula (I), wherein E is a carbon atom and wherein [symbol] is a double bond.

### Methods of Using the Compounds of the Disclosure

Compounds of formula (I) are inhibitors of Bcl-2 proteins, such as Bcl-2, and/or Bcl-xL, and thus, in some embodiments, the compounds are tool compounds useful for studying processes mediated by Bcl-2 proteins in vitro or in vivo. In vitro, the tool compounds of formula (I) may be useful for studying the effects of Bcl-2 family protein inhibition on purified proteins, cellular extracts, in intact cells and cell line models, and the like. In vivo, the tool compounds of formula (I) may be useful for studying the effects of Bcl-2 family protein inhibition in cell line derived xenografts, in patient derived xenografts, in knock-in mouse model, in knock-out mouse models, and the like.

Compounds of formula (I) are inhibitors of Bcl-2 proteins, such as Bcl-2, and/or Bcl-xL, and thus a number of diseases, conditions, or disorders mediated by Bcl-2 proteins can be treated or prevented by administering these compounds to a subject. The present disclosure is thus directed generally to a method for treating or preventing a disease, condition, or disorder responsive to the inhibition of Bcl-2 proteins, such as Bcl-2, and/or Bcl-xL, in an animal suffering from, or at risk of suffering from, the disease, condition, or disorder The method comprises administering to the animal an effective amount of one or more compounds of the disclosure.

In one embodiment, compounds of the disclosure have a Bcl-2 and/or Bcl-xL IC₅₀ of less than about 10 μM. In another embodiment, compounds of the disclosure have a Bcl-2 and/or Bcl-xL IC₅₀ of less than about 5 μM. In another embodiment, compounds of the disclosure have a Bcl-2 and/or Bcl-xL IC₅₀ of less than about 1 μM. In another embodiment, compounds of the disclosure have a Bcl-2 and/or Bcl-xL IC₅₀ of less than about 0.5 μM. In another embodiment, compounds of the disclosure have a Bcl-2 and/or Bcl-xL IC₅₀ of less than about 0.1 μM. In another embodiment, compounds of the disclosure have a Bcl-2 and/or Bcl-xL IC₅₀ of less than about 0.05 μM. In another embodiment, compounds of the disclosure have a Bcl-2 and/or Bcl-xL IC₅₀ of less than about 0.025 μM. In another embodiment, compounds of the disclosure have a Bcl-2 and/or Bcl-xL IC₅₀ of less than about 0.010 μM. In another embodiment, compounds of the disclosure have a Bcl-2 and/or Bcl-xL IC₅₀ of less than about 0.005 μM. In another embodiment, compounds of the disclosure have a Bcl-2 and/or Bcl-xL IC₅₀ of less than about 0.0025 μM. In another embodiment, compounds of the disclosure have a Bcl-2 and/or Bcl-xL IC₅₀ of less than about 0.001 μM.

The present disclosure is further directed to a method of inhibiting Bcl-2 family proteins in an animal, e.g., a human, in need thereof, the method comprising administering to the animal a therapeutically effective amount of at least one compound of the disclosure. In another embodiment, the present disclosure is directed to a method of inhibiting Bcl-2 family proteins in an animal, e.g., a human, in need thereof, the method comprising administering to the animal a therapeutically effective amount of a pharmaceutical composition comprising at least one compound of the disclosure.

The present disclosure is further directed to a method of inhibiting Bcl-2 in an animal, e.g., a human, in need thereof, the method comprising administering to the animal a therapeutically effective amount of at least one compound of the disclosure. In another embodiment, the present disclosure is directed to a method of inhibiting Bcl-2 in an animal, e.g., a human, in need thereof, the method comprising administering to the animal a therapeutically effective amount of a pharmaceutical composition comprising at least one compound of the disclosure.

The present disclosure is further directed to a method of inhibiting Bcl-xL in an animal, e.g., a human, in need thereof, the method comprising administering to the animal a therapeutically effective amount of at least one compound of the disclosure. In another embodiment, the present disclosure is directed to a method of inhibiting Bcl-xL in an animal, e.g., a human, in need thereof, the method comprising administering to the animal a therapeutically effective amount of a pharmaceutical composition comprising at least one compound of the disclosure.

In one aspect, the present disclosure provides a method of treating or preventing a disease in a subject, e.g., a human, comprising administering a therapeutically effective amount of a compound of the disclosure or a pharmaceutical composition comprising at least one compound of the disclosure. In some embodiments, the present disclosure provides a method of treating, preventing, or ameliorating a non-alcoholic fatty liver disease in a subject, e.g. a human, comprising administering a therapeutically effective amount of a compound of the disclosure or a pharmaceutical composition comprising at least one compound of the disclosure.

The present disclosure provides a method of treating or ameliorating non-alcoholic steatohepatitis in a subject, e.g. a human, comprising administering a therapeutically effective amount of a compound of the disclosure, or a pharmaceutical composition comprising at least one compound of the disclosure, to the patient in need thereof.

Patients diagnosed with non-alcoholic steatohepatitis are frequently diagnosed with a number of other common comorbidities. For example, patients suffering from non-alcoholic steatohepatitis or non-alcoholic fatty liver disease are commonly diagnosed concurrently with illnesses selected from, but not limited to, cardiovascular disease, chronic kidney disease, type 2 diabetes mellitus, obesity, and/or a metabolic syndrome, wherein the metabolic syndrome may include, but is not limited to, hypertension, hyperglycaemia, hyperlipemia, and/or insulin resistance (IR). Accordingly, in some embodiments, the present disclosure relates to a method of treating or ameliorating non-alcoholic steatohepatitis in a subject, wherein the subject is additionally diagnosed with one or more additional diseases or conditions selected from cardiovascular disease, chronic kidney disease, type 2 diabetes mellitus, obesity, and metabolic syndrome. In one embodiment, the present disclosure relates to a method of treating or ameliorating non-alcoholic steatohepatitis in a subject, wherein the subject is additionally diagnosed with cardiovascular disease. In one embodiment, the present disclosure relates to a method of treating or ameliorating non-alcoholic steatohepatitis in a subject, wherein the subject is additionally diagnosed with chronic kidney disease. In one embodiment, the present disclosure relates to a method of treating or ameliorating non-alcoholic steatohepatitis in a subject, wherein the subject is additionally diagnosed with type 2 diabetes mellitus. In one embodiment, the present disclosure relates to a method of treating or ameliorating non-alcoholic steatohepatitis in a subject, wherein the subject is additionally diagnosed with obesity. In one embodiment, the present disclosure relates to a method of treating or ameliorating non-alcoholic steatohepatitis in a subject, wherein the subject is additionally diagnosed with metabolic syndrome.

Compounds of the disclosure can be administered to a subject in the form of a raw chemical without any other components present. Compounds of the disclosure can also be administered to a subject as part of a pharmaceutical composition containing the compound combined with one or more suitable pharmaceutically acceptable carriers. Such carriers can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, Pa., 19th ed. 1995.

Pharmaceutical compositions within the scope of the present disclosure include all compositions where a compound of the disclosure is combined with one or more pharmaceutically acceptable carriers. In one embodiment, the compound of the disclosure is present in the composition in an amount that is effective to achieve its intended therapeutic purpose. While individual needs may vary, a determination of optimal ranges of effective amounts of each compound is within the skill of the art. Typically, a compound of the disclosure can be administered to a mammal, e.g., a human, orally at a dose of from about 0.0025 to about 1500 mg per kg body weight of the mammal, or an equivalent amount of a pharmaceutically acceptable salt, hydrate, solvate, stereoisomer, or tautomer thereof, per day to treat the particular disorder. A useful oral dose of a compound of the disclosure administered to a mammal is from about 0.0025 to about 200 mg per kg body weight of the mammal, or an equivalent amount of the pharmaceutically acceptable salt or solvate thereof. In some embodiments, the compound of the disclosure may be administered to a mammal at a dose of about 10 mg per kg body weight of the mammal, or an equivalent amount of the pharmaceutically acceptable salt or solvate thereof. In some embodiments, the compound of the disclosure may be administered to a mammal at a dose of about 30 mg per kg body weight of the mammal, or an equivalent amount of the pharmaceutically acceptable salt or solvate thereof. For intramuscular injection, the dose is typically about one-half of the oral dose. In some embodiments, the compound of the disclosure, or a pharmaceutically acceptable salt, hydrate, solvate, stereoisomer, or tautomer thereof is administered once per day.

A unit oral dose may comprise from about 0.01 mg to about 1 g of the compound of the disclosure, e.g., about 0.01 mg to about 500 mg, about 0.01 mg to about 250 mg, about 0.01 mg to about 100 mg, 0.01 mg to about 50 mg, e.g., about 0.1 mg to about 10 mg, of the compound. The unit dose can be administered one or more times daily, e.g., as one or more tablets or capsules, each containing from about 0.01 mg to about 1 g of the compound, or an equivalent amount of a pharmaceutically acceptable salt, hydrate, solvate, stereoisomer, or tautomer thereof.

In some embodiments, the compound of the disclosure, or a pharmaceutical composition comprising at least one compound of the disclosure, is administered to the patient in need thereof at a dose sufficient to elicit one or more effects selected from the group consisting of reduced liver steatosis, reduced lobular inflammation, reduced hepatocellular ballooning, and reduced liver fibrosis.

In one embodiment, the compound of the disclosure, or a pharmaceutical composition comprising at least one compound of the disclosure, is administered to the patient in need thereof at a dose sufficient to reduce liver steatosis in the patient.

In one embodiment, the compound of the disclosure, or a pharmaceutical composition comprising at least one compound of the disclosure, is administered to the patient in need thereof at a dose sufficient to reduce lobular inflammation in the patient.

In one embodiment, the compound of the disclosure, or a pharmaceutical composition comprising at least one compound of the disclosure, is administered to the patient in need thereof at a dose sufficient to reduce hepatocellular ballooning in the patient.

In one embodiment, the compound of the disclosure, or a pharmaceutical composition comprising at least one compound of the disclosure, is administered to the patient in need thereof at a dose sufficient to reduce liver fibrosis in the patient.

A compound of the disclosure or a pharmaceutical composition comprising a compound of the disclosure can be administered to any patient or subject that may experience the beneficial effects of a compound of the disclosure. Foremost among such patients or subject are mammals, e.g., humans and companion animals, although the disclosure is not intended to be so limited. In one embodiment, the patient or subject is a human.

A compound of the disclosure or a pharmaceutical composition comprising a compound of the disclosure can be administered by any means that achieves its intended purpose. For example, administration can be by the oral, parenteral, subcutaneous, intravenous, intramuscular, intraperitoneal, transdermal, intranasal, transmucosal, rectal, intravaginal or buccal route, or by inhalation. The dosage administered and route of administration will vary, depending upon the circumstances of the particular subject, and taking into account such factors as age, gender, health, and weight of the recipient, condition or disorder to be treated, kind of concurrent treatment, if any, frequency of treatment, and the nature of the effect desired.

In one embodiment, a compound of the disclosure or a pharmaceutical composition comprising a compound of the disclosure can be administered orally. In another embodiment, a pharmaceutical composition of the present disclosure can be administered orally and is formulated into tablets, dragees, capsules, or an oral liquid preparation. In one embodiment, the oral formulation comprises extruded multiparticulates comprising the compound of the disclosure.

Alternatively, a compound of the disclosure or a pharmaceutical composition comprising a compound of the disclosure can be administered rectally, and is formulated in suppositories.

Alternatively, a compound of the disclosure or a pharmaceutical composition comprising a compound of the disclosure can be administered by injection.

Alternatively, a compound of the disclosure or a pharmaceutical composition comprising a compound of the disclosure can be administered transdermally.

Alternatively, a compound of the disclosure or a pharmaceutical composition comprising a compound of the disclosure can be administered by inhalation or by intranasal or transmucosal administration.

Alternatively, a compound of the disclosure or a pharmaceutical composition comprising a compound of the disclosure can be administered by the intravaginal route.

A pharmaceutical composition of the present disclosure can contain from about 0.01 to 99 percent by weight, e.g., from about 0.25 to 75 percent by weight, of a compound of the disclosure, e.g., about 1%, about 5%, about 10%, about 15%, about 20%, about 25%, about 30%, about 35%, about 40%, about 45%, about 50%, about 55%, about 60%, about 65%, about 70%, or about 75% by weight of a compound of the disclosure.

A pharmaceutical composition of the present disclosure can contain one or more of a compound of formula (I). In some embodiments, the pharmaceutical composition contains Compound 1 and Compound 2 of the foregoing structures, or a pharmaceutically acceptable salt, hydrate, solvate, or tautomer thereof. In one embodiment, the pharmaceutical composition contains Compound 1 of the foregoing structure, or a pharmaceutically acceptable salt, solvate, hydrate, or tautomer thereof, at a purity of at least 90%, wherein the composition comprises less than 10%, e.g. less than 9%, less than 8%, less than 7%, less than 6%, less than 5%, less than 4%, less than 3%, less than 2% or less than 1% of Compound 2 of the foregoing structure.

A pharmaceutical composition of the present disclosure is manufactured in a manner which itself will be known in view of the instant disclosure, for example, by means of conventional mixing, granulating, dragee-making, dissolving, extrusion, or lyophilizing processes. Thus, pharmaceutical compositions for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification orin order to characterize combinations of active compound doses.

Examples of other pharmaceutical preparations that can be used orally include push-fit capsules made of gelatin, or soft, sealed capsules made of gelatin and a plasticizer such as glycerol or sorbitol. The push-fit capsules can contain a compound in the form of granules, which can be mixed with fillers such as lactose, binders such as starches, and/or lubricants such as talc or magnesium stearate and, optionally, stabilizers, or in the form of extruded multiparticulates. In soft capsules, the active compounds are preferably dissolved or suspended in suitable liquids, such as fatty oils or liquid paraffin. In addition, stabilizers can be added.

Possible pharmaceutical preparations for rectal administration include, for example, suppositories, which consist of a combination of one or more active compounds with a suppository base. Suitable suppository bases include natural and synthetic triglycerides, and paraffin hydrocarbons, among others. It is also possible to use gelatin rectal capsules consisting of a combination of active compound with a base material such as, for example, a liquid triglyceride, polyethylene glycol, or paraffin hydrocarbon.

Suitable formulations for parenteral administration include aqueous solutions of the active compound in a water-soluble form such as, for example, a water-soluble salt, alkaline solution, or acidic solution. Alternatively, a suspension of the active compound can be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

In another embodiment, the present disclosure provides kits which comprise a compound of the disclosure (or a pharmaceutical composition comprising a compound of the disclosure) packaged in a manner that facilitates their use to practice methods of the present disclosure. In one embodiment, the kit includes a compound of the disclosure (or a pharmaceutical composition comprising a compound of the disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration.

In another embodiment, a compound of the disclosure, or a pharmaceutical composition comprising a compound of the disclosure, is administered to a subject in conjunction with a second therapeutic agent. The second therapeutic agent is different from the compound of the disclosure. A compound of the disclosure and the second therapeutic agent can be administered simultaneously or sequentially to achieve the desired effect. In some embodiments, the second therapeutic agent is administered before the compound of the disclosure, or a pharmaceutical composition comprising the compound of the disclosure. In some embodiments, the second therapeutic agent is administered after the compound of the disclosure, or a pharmaceutical composition comprising the compound of the disclosure. In some embodiments, the second therapeutic agent is administered simultaneously with the compound of the disclosure, or a pharmaceutical composition comprising the compound of the disclosure. In addition, the compound of the disclosure and second therapeutic agent can be administered as a single composition or two separate compositions.

The second therapeutic agent is administered in an amount to provide its desired therapeutic effect. The effective dosage range for each second therapeutic agent is known in the art, and the second therapeutic agent is administered to an individual in need thereof within such established ranges.

A compound of the disclosure and the second therapeutic agent can be administered together as a single-unit dose or separately as multi-unit doses, wherein the compound of the disclosure is administered before the second therapeutic agent or vice versa. One or more doses of the compound of the disclosure and/or one or more dose of the second therapeutic agent can be administered. The compound of the disclosure therefore can be used in conjunction with one or more second therapeutic agents, for example, but not limited to, therapeutic agents for the treatment of non-alcoholic fatty liver disease.

In some embodiments, the second therapeutic agent is a therapeutic agent for the treatment of a non-alcoholic fatty liver disease. In some embodiments, the second therapeutic agent is a therapeutic agent for the treatment of a non-alcoholic steatohepatitis. For example, therapeutic agents for the treatment of non-alcoholic steatohepatitis, or currently under investigation for the treatment of non-alcoholic steatohepatitis, may include, but are not limited to, lipase inhibitors, microbiome modulators, PPAR agonists, GLP-1/DPP-4 agents, FXR-bile acid agents, anti-lipid agents, anti-inflammatory therapeutics, anti-caspase agents, anti-oxidants, THR-β agonists and antifibrotic agents.

Non-limiting examples of lipase inhibitors for use in the treatment of non-alcoholic steatosis include orlistat, a lipase inhibitor for the treatment of obesity that functions by reducing dietary fat absorption.

Non-limiting examples of microbiome modulators for use in the treatment of non-alcoholic steatosis include macrolide antibiotics, such as solithromycin, which may modulate the gut flora of the patient to reduce the release of bacterially produced biomolecular products such as lipopolysaccharides.

Non-limiting examples of PPAR agonists for use in the treatment of non-alcoholic steatosis include elafibranor, also known as GFT505, pioglitazone, and saroglitazar.

Non-limiting examples of GLP-1/DDP-4 agents for use in the treatment of non-alcoholic steatosis include exenatide, liraglutide, sitagliptin, and vildagliptin.

Non-limiting examples of FXR-bile acid agents for use in the treatment of non-alcoholic steatosis include obeticholic acid (OCA) and fibroblast growth factoru 19 (FGF-19).

Non-limiting examples of anti-lipid agents for use in the treatment of non-alcoholic steatosis include aramchol and statins, such as atorvastatin, fluvastatin, lovastatin, pitavastatin, pravastatin, rosuvastatin, and simvastatin

Non-limiting examples of anti-inflammatory therapeutics for use in the treatment of non-alcoholic steatosis include cenicriviroc and pentoxifylline.

Non-limiting examples of anti-caspase agents for use in the treatment of non-alcoholic steatosis include emricasan.

Non-limiting examples of anti-oxidant agents for use in the treatment of non-alcoholic steatosis include vitamin E and pentoxifylline.

Non-limiting examples of THR-β agonists for use in the treatment of non-alcoholic steatosis include resmetirom (MGL-3196) and ASC41.

Non-limiting examples of antifibrotic agents for use in the treatment of non-alcoholic steatosis include simtuzumab and belapectin, also known as GR-MD-02.

The above-mentioned second therapeutically active agents, one or more of which can be used in combination with a compound of the disclosure, are prepared and administered as described in the art.

In some embodiments, the second therapeutic agent is obeticholic acid. In some embodiments, the obeticholic acid is administered at a dose of about 0.1 to about 100 mg/kg to the patient in need thereof. In some embodiments, the obeticholic acid is administered at a dose of about 30 mg/kg to the patient in need thereof. In one embodiment, the obeticholic acid is administered before the compound of the disclosure. In one embodiment, the obeticholic acid is administered concurrently with the compound of the disclosure. In one embodiment, the obeticholic acid is administered concurrently with the compound of the disclosure, wherein the compound of the disclosure and obtecholic acid are administered in a single pharmaceutical composition. In one embodiment, the obeticholic acid is administered after the compound of the disclosure. In one embodiment, the obeticholic acid is administered at a dose of about 30 mg/kg and the compound of the disclosure is administered at a dose of about 50 mg/kg.

Given the relationship between body mass index and non-alcoholic steatohepatitis, patients diagnosed with non-alcoholic steatohepatitis commonly are instructed to modify their diets by reducing caloric intake and to maintain a regular physical exercise regimen. Accordingly, in some embodiments, patients to be treated for non-alcoholic steatohepatitis using the methods disclosed herein may also be consuming reduced calorie diets and/or may be maintaining a regular physical exercise regimen.

### EXAMPLES

### Example 1—Synthesis of Intermediates

### Intermediate 1

### Synthesis of 1-cyclobutylidenepropan-2-one

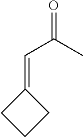

To a solution of cyclobutanone (5.0 g, 71.4 mmol) in toluene (200 ml) was added 1-(triphenylphosphoranylidene)-2-propanone (22.7 g, 71.4 mmol) and the mixture was refluxed overnight. Solvent was removed under reduced pressure and the residue was purified by silica gel column chromatography (ethyl acetate/hexane 1/10-1/5) to afford 1-cyclobutylidenepropan-2-one (5.0 g) as yellow oil. ¹H NMR (400 MHz, CDCl₃) δ 5.95-5.93 (m, 1H), 3.19-3.13 (m, 2H), 2.91-2.84 (m, 2H), 2.21 (s, 3H), 2.21-2.11 (m, 2H).

### Intermediate 2

### Synthesis of Spiro[3.5]nonane-6,8-dione

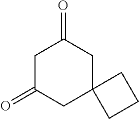 [SMILES: O=C1CC(=O)CC2(CCC2)C1]

To a solution of 1-cyclobutylidenepropan-2-one (23.1 g, 0.21 mol) and methyl malonate (30.3 g, 0.23 mol) in methanol (150 ml) was added sodium methoxide (41 0.4 g, 30% in methanol). The mixture was heated to reflux under nitrogen for 4 h and concentrated. The resulting residue was hydrolyzed in 2N potassium hydroxide (200 ml) at 70° C. for 4 h. The mixture was extracted with ethyl acetate (100 ml), then titrated to pH 3-5 with 1N hydrochloric acid. The resulting solution was heated to 70° C. for 5 h and extracted with ethyl acetate (100 ml×3). The combined organic layers were dried over magnesium sulfate and concentrated to afford spiro[3.5]nonane-6,8-dione (19.8 g, 62.3%) as yellow solid. This product was used directly in the next step without further purification. ¹H NMR (400 MHz, DMSO-d6) δ 11.05 (s, 1H), 5.17 (s, 1H), 2.50-2.35 (m, 4H), 1.92-1.79 (m, 2H), 1.79-1.72 (m, 4H).

### Intermediate 3

### 8-Isobutoxyspiro[3.5]non-7-en-6-one

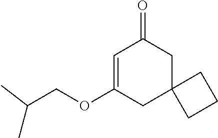

To a solution of spiro[3.5]nonane-6,8-dione (19.8 g, 0.13 mol) in toluene (150 ml) was added 4-toluenesulfonic acid (248 mg, 0.0013 mol) and iso-butyl alcohol (14.5 g, 0.2 mol). The mixture was heated to reflux and water was removed by azeotropic distillation. Solvent was removed under vacuum and the residue was purified by silica gel column chromatography (ethyl acetate/petrol ether 1/10-1/3) to afford 8-isobutoxyspiro[3.5]non-7-en-6-one (25.0 g, 92.7%) as light yellow oil. ¹H NMR (400 MHz, CDCl₃) δ 5.31 (s, 1H), 3.59 (d, J=6.8 Hz, 2H), 2.51 (s, 2H), 2.45 (s, 2H), 2.12-1.96 (m, 1H), 1.93-1.83 (m, 6H), 0.99 (d, J=6.8 Hz, 6H).

### Intermediate 4

### Synthesis of Spiro[3.5]non-7-en-6-one

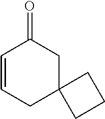

To a solution of 8-isobutoxyspiro[3.5]non-7-en-6-one (25.0 g, 0.12 mol) in toluene (100 mL) was added Red-Al® (40 ml, 70% in toluene, 0.18 mol) dropwise at room temperature. The mixture was heated to 45° C. for 4 h, then quenched by 1N hydrochloric acid. The mixture was filtered and the filtrate was concentrated and purified by silica gel column chromatography (ethyl acetate/petrol ether 1/10) to afford spiro[3.5]non-7-en-6-one (9.0 g, 55%) as light yellow oil.

### Intermediate 5

### Synthesis of Spiro[3.5]nonan-6-one

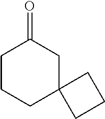

Spiro[3.5]non-7-en-6-one (9.0 g) was hydrogenated under 1 atm hydrogen, catalyzed by 10% Pd/C (1.0 g) in methanol (80 ml) for 5.5 h. Pd/C was removed by filtration and the filtrate was concentrated to afford spiro[3.5]nonan-6-one (8.8 g, 96.4%) as colorless oil which was used directly in the next step without further purification. ¹H NMR (400 MHz, CDCl₃) δ 2.38 (s, 2H), 2.23-2.20 (m, 2H), 1.89-1.75 (m, 10H).

### Intermediate 6

### Synthesis of Methyl 6-oxospiro[3.5]nonane-7-carboxylate

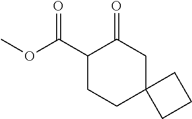

To a suspension of sodium hydride (5.1 g, 0.13 mol) in tetrahydrofuran (150 mL) was added methyl carbonate (28.7 g, 0.32 mol) at room temperature, followed by spiro[3.5]nonan-6-one in tetrahydrofuran (30 mL). The mixture was refluxed for 2 h. The reaction was quenched by saturated aqueous ammonium chloride and extracted with ethyl acetate (100 mL×3). The combined organic layers were washed with brine and concentrated. The resulting residue was purified by silica gel column chromatography to afford methyl 6-oxospiro[3.5]nonane-7-carboxylate (4.0 g, 32%) as light yellow oil.

### Intermediate 7

### Synthesis of Methyl 6-(((trifluoromethyl)sulfonyl)oxy)spiro[3.5]non-6-ene-7-carboxylate

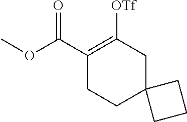 [SMILES: COC(=O)C1=C(OS(=O)(=O)C(F)(F)F)CC2(CCC2)CC1]

To a solution of methyl 6-oxospiro[3.5]nonane-7-carboxylate (4.0 g, 0.02 mol) in tetrahydrofuran (25 mL) were added potassium carbonate (5.6 g, 0.04 mol) and N,N-bis(trifluoromethylsulfonyl)aniline (7.9 g, 0.022 mol). The mixture was stirred at room temperature overnight, diluted with water, and extracted with ethyl acetate (100 mL×3). The combined organic layers were washed with saturated brine, dried over magnesium sulfate and concentrated. The resulting residue was purified by silica gel column chromatography (ethyl acetate/petrol ether 1/50-1/10) to afford methyl 6-(((trifluoromethyl)sulfonyl)oxy)spiro[3.5]non-6-ene-7-carboxylate (5.0 g, 76%) as light yellow oil.

### Intermediate 8

### Synthesis of Methyl 6-(4-chlorophenyl)spiro[3.5]non-6-ene-7-carboxylate

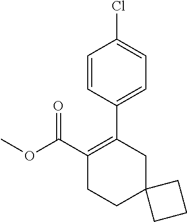 [SMILES: COC(=O)C1=C(c2ccc(Cl)cc2)CC2(CCC2)CC1]

The mixture of methyl 6-(((trifluoromethyl)sulfonyl)oxy)spiro[3.5]non-6-ene-7-carboxylate (5.0 g, 0.015 mol), 4-chlorophenyl boronic acid (2.58 g, 0.017 mol), CsF (4.63 g, 0.03 mol) and Pd(PPh₃)₄ (173 mg, 0.15 mol) in 1,2-dimethoxy-ethane (30 ml) and methanol (15 ml) was heated to 70° C. under nitrogen for 2 h. Solvents were removed under reduced pressure and the residue was purified by silica gel column chromatography (ethyl acetate/petrol ether 1/10) to afford methyl 6-(4-chlorophenyl)spiro[3.5]non-6-ene-7-carboxylate (4.0 g, 92%) as colorless oil. ¹H NMR (400 MHz, CDCl₃) δ 7.30 (d, J=8.5 Hz, 2H), 7.06 (d, J=8.5 Hz, 2H), 3.48 (s, 3H), 2.50-2.44 (m, 2H), 2.43 (t, J=2.3 (2.3 or 6.3?) Hz, 2H), 2.02-1.80 (m, 6H), 1.74 (t, J=6.3 Hz, 2H).

### Intermediate 9

### Synthesis of (6-(4-Chlorophenyl)spiro[3.5]non-6-en-7-yl)methanol

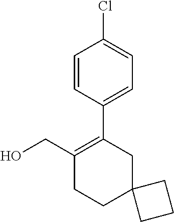 [SMILES: OCC1=C(c2ccc(Cl)cc2)CC2(CCC2)CC1]

To a solution of methyl 6-(4-chlorophenyl)spiro[3.5]non-6-ene-7-carboxylate (4.0 g, 0.014 mol) in tetrahydrofuran (20 mL) was added a solution of LiBH₄ (910 mg, 0.042 mol) in tetrahydrofuran (10 mL). The mixture was stirred at room temperature overnight, quenched by 1N aqueous hydrochloric acid and extracted with ethyl acetate (100 mL×3). The combined organic layers were washed with brine, dried over magnesium sulfate and concentrated. The resulting residue was purified by silica gel column chromatography (ethyl acetate/petrol ether 1/10-1/3) to afford (6-(4-chlorophenyl)spiro[3.5]non-6-en-7-yl)methanol (3.0 g, 81.7%) as a white solid. ¹H NMR (400 MHz, CDCl₃) δ 7.31 (d, J=8.4 Hz, 2H), 7.09 (d, J=8.4 Hz, 2H), 3.93 (d, J=4.2 Hz, 2H), 2.37-2.26 (m, 2H), 2.01-1.77 (m, 8H), 1.74 (t, J=6.3 Hz, 2H).

### Intermediate 10

### Synthesis of 7-(Chloromethyl)-6-(4-chlorophenyl)spiro[3.5]non-6-ene

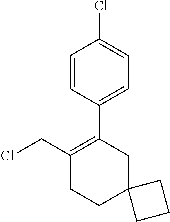 [SMILES: ClCC1=C(c2ccc(Cl)cc2)CC2(CCC2)CC1]

To a solution of (6-(4-chlorophenyl)spiro[3.5]non-6-en-7-yl)methanol (3.5 g, 0.013 mol) and trimethylamine (2.7 g, 0.026 mol) in dichloromethane (20 mL) was added methylsulfonyl chloride (3.0 g, 0.026 mol) dropwise. The mixture was stirred at room temperature for 5 h. Solvent was removed under reduced pressure and the residue was purified by silica gel column chromatography to afford 7-(chloromethyl)-6-(4-chlorophenyl)spiro[3.5]non-6-ene (2.75 g, 75.5%) as yellow oil. ¹H NMR (400 MHz, CDCl₃) δ 7.31 (d, J=8.4 Hz, 2H), 7.09 (d, J=8.5 Hz, 2H), 3.93 (s, 2H), 2.34-2.25 (m, 4H), 1.97-1.78 (m, 6H), 1.74 (t, J=6.3 Hz, 2H).

### Intermediate 11

### Synthesis of Methyl 2-((1H-pyrrolo[2,3-b]pyridin-5-yl)oxy)-4-bromobenzoate

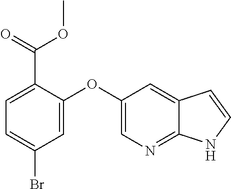 [SMILES: COC(=O)c1ccc(Br)cc1Oc1cnc2[nH]ccc2c1]

A mixture of 1-(triisopropylsilyl)-1H-pyrrolo[2,3-b]pyridin-5-ol (1.91 g), methyl 4-bromo-2-fluorobenzoate (1.70 g), and K3P04 (1.86 g) in diglyme (20 mL) was stirred at 115° C. for 1 h. The reaction was cooled, diluted with ethyl acetate (100 mL), washed with water followed by brine, and concentrated. The residue was purified by silica gel chromatography (ethyl acetate/hexane 1/3) to afford methyl 2-((1H-pyrrolo[2,3-b]pyridin-5-yl)oxy)-4-bromobenzoate (1.8 g) as white solid. ¹H NMR (400 MHz, CDCl₃) δ 9.28 (s, 1H), 8.18 (d, J=2.5 Hz, 1H), 7.79 (d, J=8.4 Hz, 1H), 7.62 (d, J=2.5 Hz, 1H), 7.40-6.96 (m, 2H), 6.96 (d, J=1.7 Hz, 1H), 6.51-6.48 (m, 1H), 3.89 (s, 3H).

### Intermediate 12

### Synthesis of 2-((1H-pyrrolo[2,3-b]pyridin-5-yl)oxy)-4-bromobenzoic acid

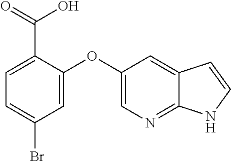 [SMILES: O=C(O)c1ccc(Br)cc1Oc1cnc2[nH]ccc2c1]

To a solution of methyl 2-((1H-pyrrolo[2,3-b]pyridin-5-yl)oxy)-4-bromobenzoate (300 mg, 0.867 mmol) in dioxane (10 mL) was added 1N NaOH (2.2 mL, 2.2 mmol) and the mixture was stirred at room temperature for 2 h. The mixture was acidified by 1N HCl and extracted with ethyl acetate, washed with brine, and dried over anhydrous MgSO₄. Evaporation under reduced pressure afforded crude 2-((1H-pyrrolo[2,3-b]pyridin-5-yl)oxy)-4-bromobenzoic acid as a colorless oil. This product was used directly in the next step without further purification.

### Intermediate 13

### Synthesis of 2-((1H-pyrrolo[2,3-b]pyridin-5-yl)oxy)-4-bromo-N-((3-nitro-4-(((tetrahydro-2H-pyran-4-yl)methyl)amino)phenyl)sulfonyl) benzamide

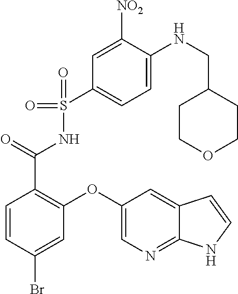 [SMILES: O=C(NS(=O)(=O)c1ccc(NCC2CCOCC2)c([N+](=O)[O-])c1)c1ccc(Br)cc1Oc1cnc2[nH]ccc2c1]

To a solution of 2-((1H-pyrrolo[2,3-b]pyridin-5-yl)oxy)-4-bromobenzoic acid (100 mg, 0.3 mmol) in DCM (10 mL) were added 3-nitro-4-(((tetrahydro-2H-pyran-4-yl)methyl)amino)benzenesulfonamide (95 mg, 0.3 mmol), DMAP (55 mg, 0.45 mmol) and EDCI (115 mg, 0.6 mmol) and the mixture was stirred at room temperature for 24 h. Solvent was removed under reduced pressure and the residue was purified by silica gel chromatography (DCM/MeOH 95/5) to afford 2-((1H-pyrrolo[2,3-b]pyridin-5-yl)oxy)-4-bromoN-((3-nitro-4-(((tetrahydro-2H-pyran-4-yl)methyl)amino)phenyl) sulfonyl)benzamide as a yellow oil (80 mg). MS m/z 630 [M+H]⁺.

### Intermediate 14

### Synthesis of (S)—N-((4-(((1,4-Dioxan-2-yl)methyl)amino)-3-nitrophenyl)sulfonyl)-2-((1H-pyrrolo[2,3-b]pyridin-5-yl)oxy)-4-bromobenzamide

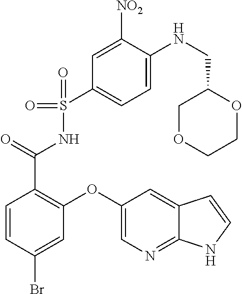 [SMILES: O=C(NS(=O)(=O)c1ccc(NC[C@H]2COCCO2)c([N+](=O)[O-])c1)c1ccc(Br)cc1Oc1cnc2[nH]ccc2c1]

To a solution of 2-((1H-pyrrolo[2,3-b]pyridin-5-yl)oxy)-4-bromobenzoic acid (100 mg, 0.3 mmol) in DCM (10 mL) were added (S)-4-(((1,4-dioxan-2-yl)methyl)amino)-3-nitrobenzenesulfonamide (95 mg, 0.3 mmol), DMAP (55 mg, 0.45 mmol) and EDCI (115 mg, 0.6 mmol) and the mixture was stirred at room temperature for 24 h. Solvent was removed under reduced pressure and the residue was purified by silica gel chromatography (DCM/MeOH 95/5) to afford (S)—N-((4-(((1,4-Dioxan-2-yl)methyl)amino)-3-nitrophenyl)sulfonyl)-2-((1H-pyrrolo[2,3-b]pyridin-5-yl)oxy)-4-bromobenzamide. ¹H NMR (400 MHz, DMSO-d6) δ 11.79 (s, 1H), 8.59-8.52 (m, 2H), 8.05 (d, J=2.6 Hz, 1H), 7.85 (dd, J=9.2, 2.4 Hz, 1H), 7.66 (d, J=2.6 Hz, 1H), 7.59-7.49 (m, 1H), 7.48 (d, J=8.2 Hz, 1H), 7.34 (dd, J=8.2, 1.8 Hz, 1H), 7.12 (d, J=9.2 Hz, 1H), 6.88 (d, J=1.8 Hz, 1H), 6.50-6.40 (m, 1H), 3.83-3.37 (m, 2H), 3.72-3.56 (m, 2H), 3.56-3.42 (m, 2H), 3.37-3.01 (m, 3H).

### Intermediate 15

### Synthesis of (R)—N-((4-(((1,4-dioxan-2-yl)methyl)amino)-3-nitrophenyl)sulfonyl)-2-((1H-pyrrolo[2,3-b]pyridin-5-yl)oxy)-4-bromobenzamide

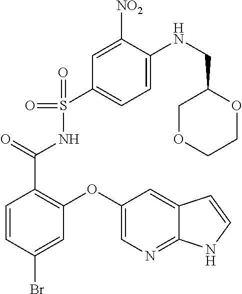 [SMILES: O=C(NS(=O)(=O)c1ccc(NC[C@@H]2COCCO2)c([N+](=O)[O-])c1)c1ccc(Br)cc1Oc1cnc2[nH]ccc2c1]

To a solution of 2-((1H-pyrrolo[2,3-b]pyridin-5-yl)oxy)-4-bromobenzoic acid (100 mg, 0.3 mmol) in DCM (10 mL) were added (R)-4-(((1,4-dioxan-2-yl)methyl)amino)-3-nitrobenzenesulfonamide (95 mg, 0.3 mmol), DMAP (55 mg, 0.45 mmol) and EDCI (115 mg, 0.6 mmol) and the mixture was stirred at room temperature for 24 h. Solvent was removed under reduced pressure and the residue was purified by silica gel chromatography (DCM/MeOH 95/5) to afford (R)—N-((4-(((1,4-dioxan-2-yl)methyl)amino)-3-nitrophenyl)sulfonyl)-2-((1H-pyrrolo[2,3-b]pyridin-5-yl)oxy)-4-bromobenzamide. ¹H NMR (400 MHz, DMSO-d6) δ 11.79 (s, 1H), 8.59-8.52 (m, 2H), 8.05 (d, J=2.6 Hz, 1H), 7.85 (dd, J=9.2, 2.4 Hz, 1H), 7.66 (d, J=2.6 Hz, 1H), 7.59-7.49 (m, 1H), 7.48 (d, J=8.2 Hz, 1H), 7.34 (dd, J=8.2, 1.8 Hz, 1H), 7.12 (d, J=9.2 Hz, 1H), 6.88 (d, J=1.8 Hz, 1H), 6.50-6.40 (m, 1H), 3.83-3.37 (m, 2H), 3.72-3.56 (m, 2H), 3.56-3.42 (m, 2H), 3.37-3.01 (m, 3H).

### Intermediate 16

### Synthesis of tert-Butyl-4-(3-((1H-pyrrolo[2,3-b]pyridin-5-yl)oxy)-4-(ethoxycarbonyl)phenyl)piperazine-1-carboxylate

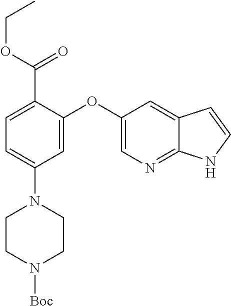 [SMILES: CCOC(=O)c1ccc(N2CCN(C(=O)OC(C)(C)C)CC2)cc1Oc1cnc2[nH]ccc2c1]

A mixture of ethyl 2-((1H-pyrrolo[2,3-b]pyridin-5-yl)oxy)-4-fluorobenzoate (2.1 g, 7 mmol), N-Boc-piperazine (2.61 g, 0.014 mol) and dipotassium hydrogenphosphate (2.44 g, 0.014 mol) in dimethyl sulfoxide was heated to 135° C. overnight. Water was added to the reaction mixture and the mixture was extracted with ethyl acetate (50 mL×3). The combined organic layers were washed with brine, concentrated and purified by silica gel column chromatography to afford tert-butyl 4-(3-((1H-pyrrolo[2,3-b]pyridin-5-yl)oxy)-4-(ethoxycarbonyl)phenyl)piperazine-1-carboxylate (2.4 g, 73%) as a white solid. ¹H NMR (400 MHz, CDCl₃) δ 9.42 (br s, 1H), 8.20 (d, J=2.5 Hz, 1H), 7.95 (d, J=8.9 Hz, 1H), 7.53 (d, J=2.5 Hz, 1H), 7.37 (dd, J=3.5, 2.5 Hz, 1H), 6.66 (dd, J=8.9, 2.5 Hz, 1H), 6.46 (dd, J=3.5, 2.0 Hz, 1H), 6.36 (d, J=2.5 Hz, 1H), 4.28 (q, J=7.1 Hz, 2H), 3.55-3.50 (m, 4H), 3.21-3.17 (m, 4H), 1.47 (s, 9H), 1.26 (t, J=7.1 Hz, 3H).

### Intermediate 17

### Synthesis of Ethyl 2-((1H-pyrrolo[2,3-b]pyridin-5-yl)oxy)-4-(piperazin-1-yl)benzoate

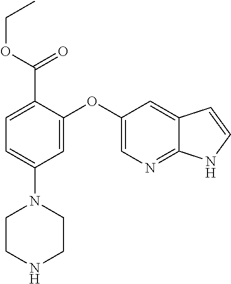 [SMILES: CCOC(=O)c1ccc(N2CCNCC2)cc1Oc1cnc2[nH]ccc2c1]

Trifluoroacetic acid (6 mL) was added to a solution of tert-butyl 4-(3-((1H-pyrrolo[2,3-b]pyridin-5-yl)oxy)-4-(ethoxycarbonyl)phenyl)piperazine-1-carboxylate (2.1 g) in dichloromethane (10 mL) and the mixture was stirred at room temperature for 3 h. Solvent was removed under reduced pressure and the crude ethyl 2-((1H-pyrrolo[2,3-b]pyridin-5-yl)oxy)-4-(piperazin-1-yl)benzoate (2.5 g) was used directly in the next step without further purification.

### Intermediate 18

### Synthesis of Ethyl 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoate

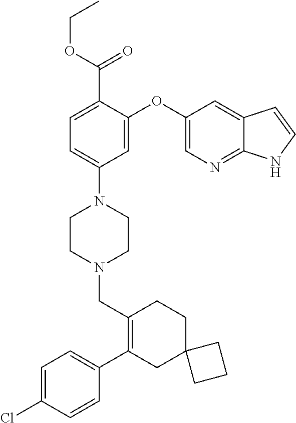 [SMILES: CCOC(=O)c1ccc(N2CCN(CC3=C(c4ccc(Cl)cc4)CC4(CCC4)CC3)CC2)cc1Oc1cnc2[nH]ccc2c1]

To a solution of 7-(chloromethyl)-6-(4-chlorophenyl)spiro[3.5]non-6-ene (851 mg, 3 mmol) in N,N-dimethyl formamide (10 mL) were added potassium carbonate (1.26 g, 9 mmol), potassium iodide (100 mg, 0.6 mmol) and ethyl 2-((1H-pyrrolo[2,3-b]pyridin-5-yl)oxy)-4-(piperazin-1-yl)benzoate (1.53 g, 3.3 mmol). The mixture was stirred at room temperature overnight. The reaction mixture was diluted with water and extracted with ethyl acetate (50 mL×3). The combined organic layers were washed with brine, concentrated and purified by silica gel column chromatography (ethyl acetate/petrol ether 1/5-1/1) to afford ethyl 2-((1H-pyrrolo[2,3-b]pyridin-5-yl)oxy)-4-(4-((6-(4-chlorophenyl)spiro[3.5]non-6-en-7-yl) methyl)piperazin-1-yl)benzoate (1.3 g, 71%) as white solid. ¹H NMR (400 MHz, CDCl₃) δ 9.98 (s, 1H), 8.20 (d, J=2.6 Hz, 1H), 7.91 (d, J=9.0 Hz, 1H), 7.51 (d, J=2.6 Hz, 1H), 7.38 (t, J=3.5 Hz, 1H), 7.28 (d, J=8.3 Hz, 2H), 6.97 (d, J=8.3 Hz, 2H), 6.62 (dd, J=9.0, 2.5 Hz, 1H), 6.45 (dd, J=3.5, 2.0 Hz, 1H), 6.32 (d, J=2.5 Hz, 1H), 4.26 (q, J=7.1 Hz, 2H), 3.20-3.12 (m, 4H), 2.77 (s, 2H), 2.31-2.17 (m, 8H), 1.98-1.72 (m, 6H), 1.68 (t, J=6.3 Hz, 2H), 1.25 (t, J=7.1 Hz, 3H).

### Intermediate 19

### Synthesis of 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoic acid

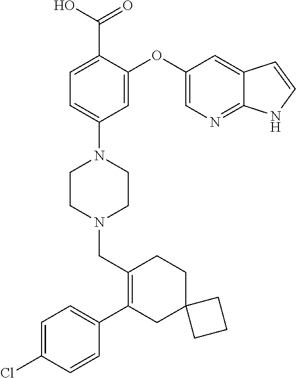 [SMILES: O=C(O)c1ccc(N2CCN(CC3=C(c4ccc(Cl)cc4)CC4(CCC4)CC3)CC2)cc1Oc1cnc2[nH]ccc2c1]

The solution of ethyl 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoate (1 0.3 g, 2.1 mmol) and 2N potassium hydroxide (12 mL, 0.042 mol) in dioxane (15 mL) was heated to 60° C. overnight. The mixture was neutralized with 1N aqueous hydrochloric acid to pH 7 and extracted with ethyl acetate (50 mL×3). The combined organic layers were washed with brine, dried over magnesium sulfate, and concentrated to afford 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoic acid (1.1 g, 88.7%) as a white solid. ¹H NMR (400 MHz, CDCl₃) δ 10.34 (s, 1H), 8.19 (d, J=2.6 Hz, 1H), 8.02 (d, J=9.0 Hz, 1H), 7.63 (d, J=2.6 Hz, 1H), 7.38-7.34 (m, 1H), 7.27 (d, J=8.3 Hz, 2H), 6.96 (d, J=8.3 Hz, 2H), 6.63 (dd, J=9.0, 2.4 Hz, 1H), 6.44 (dd, J=3.5, 1.5 Hz, 1H), 6.22 (d, J=2.4 Hz, 1H), 3.81 (s, 2H), 3.17-3.10 (m, 4H), 2.80 (s, 2H), 2.30-2.20 (m, 6H), 1.98-1.72 (m, 6H), 1.67 (t, J=6.3 Hz, 2H).

### Intermediate 20

### Synthesis of 1-(Oxetan-3-ylidene)propan-2-one

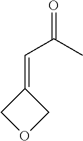

To a solution of oxetan-3-one (20.6 g, 0.28 mol) in DCM (300 ml) was added 1-(triphenylphosphoranylidene)propan-2-one (98.6 g, 0.31 mol). The mixture was stirred at room temperature overnight. DCM was removed under reduced pressure until solid was precipitated. The solid was removed by filtration and the filtrate was concentrated and purified by silica gel column chromatography (ethyl acetae/heptane 1/5-1/3) to afford 1-(oxetan-3-ylidene)propan-2-one (23.3 g, 74.3%) as a yellow oil.

### Intermediate 21

### Synthesis of 2-Oxaspiro[3.5]nonane-6,8-dione

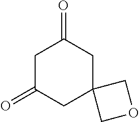 [SMILES: O=C1CC(=O)CC2(COC2)C1]

To a solution of 1-(oxetan-3-ylidene)propan-2-one (23.3 g, 0.21 mol) and methyl malonate (30.2 g, 0.23 mol) in methanol (150 ml) was added sodium methoxide (41.3 g, 30% MeOH solution). The mixture was heated to reflux under nitrogen for 1 h. Solvent was removed under reduced pressure to afford methyl 6-hydroxy-8-oxo-2-oxaspiro[3.5]non-6-ene-5-carboxylate which was used in the next step directly without purification. To an aqueous solution of KOH (2 mol/L, 200 ml) was added methyl 6-hydroxy-8-oxo-2-oxaspiro[3.5]non-6-ene-5-carboxylate. After stirring at room temperature for 30 min, the aqueous solution was extracted with ethyl acetate (150 ml×3). The aqueous layer was adjusted to pH 3-5 with 1 N hydrochloric acid and heated at 50° C. for 4 h. Water was removed under reduced pressure and the residue was purified by silica gel column chromatography to afford 2-oxaspiro[3.5]nonane-6,8-dione (2.5 g, 77.0%) as light yellow solid. This product was used directly in the next step without further purification.

### Intermediate 22

### Synthesis of 8-Isobutoxy-2-oxaspiro[3.5]non-7-en-6-one

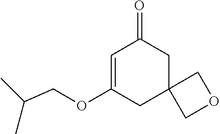

To a solution of 2-oxaspiro[3.5]nonane-6,8-dione (25 g, 0.16 mol) in toluene (150 ml) were added TsOH (238 mg, 0.0016 mol) and isobutyl alcohol (18 g, 0.24 mol). The reaction was completed after stirring at room temperature for 1 h. Solvent was removed under reduced pressure and the residue was purified by silica gel column chromatography (ethyl acetate/petrol ether 1/5-1/3) to afford 8-isobutoxy-2-oxaspiro[3.5]non-7-en-6-one (6 g, 43%) as light yellow oil. ¹H NMR (400 MHz, CDCl₃) δ 5.34 (s, 1H), 4.47 (d, J=6.1 Hz, 2H), 4.45 (d, J=6.1 Hz, 2H), 3.60 (d, J=6.8 Hz 2H), 2.80 (s, 2H), 2.68 (s, 2H), 2.09-2.01 (m, 1H), 0.98 (d, J=6.8 Hz, 6H).

### Intermediate 23

### Synthesis of 2-Oxaspiro[3.5]non-7-en-6-one

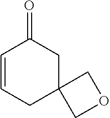

To a solution of 8-isobutoxy-2-oxaspiro[3.5]non-7-en-6-one (14.7 g, 0.07 mol) in toluene (100 ml) was added Red-Al® (40.4 g, 70% in Toluene) dropwise. The mixture was heated to 45° C. for 2 h and quenched by 1 N HCl solution. The mixture was concentrated and purified by silica gel column chromatography (ethyl acetae/petrol ether 1/10-1/5) to afford 2-oxaspiro[3.5]non-7-en-6-one (8.8 g, 91%) as colorless oil. This product was used directly in the next step without further purification.

### Intermediate 24

### Synthesis of 2-Oxaspiro[3.5]nonan-6-one

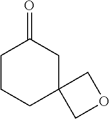

To a solution of 2-oxaspiro[3.5]non-7-en-6-one (8.8 g) in tetrahydrofuran (80 ml) was added Pd/C (1 g). The mixture was hydrogenated under 1 atm hydrogen at room temperature for 2 h. After the reaction was completed, Pd/C was removed by filtration and the solution was concentrated to afford 2-oxaspiro[3.5]nonan-6-one (8.0 g, 89.6%) as colorless oil. This product was used directly in the next step without further purification.

### Intermediate 25

### Synthesis of Methyl 6-oxo-2-oxaspiro[3.5]nonane-7-carboxylate

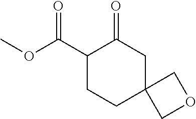

To a suspension of sodium hydride (4.6 g, 0.11 mol) in tetrahydrofuran (150 ml) under nitrogen was added methyl carbonate (25.7 g, 0.28 mol) dropwise. After dropping was completed, the mixture was heated to reflux. A solution of 2-oxaspiro[3.5]nonan-6-one (11.2 g, 0.057 mol) in tetrahydrofuran (30 ml) was then added. The reaction was heated at reflux for 2 h and quenched by saturated aqueous ammonium chloride, and extracted with ethyl acetate (100 ml×3). The combined organic layer was washed with brine, concentrated under reduced pressure and the residue was purified by silica gel column chromatography to afford methyl 6-oxo-2-oxaspiro[3.5]nonane-7-carboxylate (4.5 g, 69%) as colorless oil. This product was used directly in the next step without further purification.

### Intermediate 26

### Synthesis of Methyl 6-(((trifluoromethyl)sulfonyl)oxy)-2-oxaspiro[3.5]non-6-ene-7-carboxylate

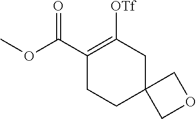 [SMILES: COC(=O)C1=C(OS(=O)(=O)C(F)(F)F)CC2(CC1)COC2]

To a suspension of methyl 6-oxo-2-oxaspiro[3.5]nonane-7-carboxylate (4.5 g, 0.02 mol) and potassium carbonate (6.3 g, 0.046 mol) in DMF (30 ml) was added N,N-bis(trifluoromethylsulfonyl)aniline (8.9 g, 0.025 mol). The mixture was stirred at room temperature overnight, diluted with water, and extracted with ethyl acetate (100 ml×3). The combined organic layer was washed with brine, dried over MgSO4, and concentrated under reduced pressure. The residue was purified by silica gel column chromatography (ethyl acetate/Petrol ether 1/10-1/3) to afford methyl 6-(((trifluoromethyl)sulfonyl)oxy)-2-oxaspiro[3.5]non-6-ene-7-carboxylate (6.6 g, 86%) as light yellow oil. This product was used directly in the next step without further purification.

### Intermediate 27

### Synthesis of Methyl 6-(4-chlorophenyl)-2-oxaspiro[3.5]non-6-ene-7-carboxylate

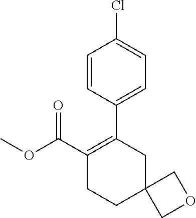 [SMILES: COC(=O)C1=C(c2ccc(Cl)cc2)CC2(CC1)COC2]

To a solution of methyl 6-(((trifluoromethyl)sulfonyl)oxy)-2-oxaspiro[3.5]non-6-ene-7-carboxylate (6.6 g, 0.02 mol) in 1,2-dimethoxy-ethan (30 ml) and methanol (10 ml) were added 4-chloro-phenyl boronic acid (3.13 g, 0.02 mol), CsF (6.08 g, 0.04 mol) and Pd(PPh₃)₄ (231 mg, 0.2 mmol) and the mixture was heated to 70° C. under nitrogen for 30 min. Solvents were removed under reduced pressure and the residue was purified by silica gel column chromatography (ethyl acetate/petrol ether 1/5-1/3) to afford methyl 6-(4-chlorophenyl)-2-oxaspiro[3.5]non-6-ene-7-carboxylate (5.1 g, 87.3%) as light yellow solid. ¹H NMR (400 MHz, CDCl₃) δ 7.33 (d, J=8.4 Hz, 2H), 7.07 (d, J=8.4 Hz, 2H), 4.54 (d, J=5.6 Hz, 2H), 4.48 (d, J=5.6 Hz, 2H), 3.48 (s, 3H), 2.74-2.70 (m, 2H), 2.55-2.50 (m, 2H), 2.04 (t, J=6.4 Hz, 2H).

### Intermediate 28

### Synthesis of (6-(4-Chlorophenyl)-2-oxaspiro[3.5]non-6-en-7-yl)methanol

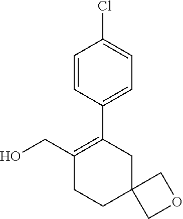 [SMILES: OCC1=C(c2ccc(Cl)cc2)CC2(CC1)COC2]

To a solution of methyl 6-(4-chlorophenyl)-2-oxaspiro[3.5]non-6-ene-7-carboxylate (2.1 g, 0.0072 mol) in tetrahydrofuran (20 ml) was added LiBH₄ (475 mg, 0.022 mol) in tetrahydrofuran (10 ml) dropwise at room temperature. The mixture was stirred at room temperature for 4 h, quenched by 1 N HCl solution, and extracted with ethyl acetate (100 ml×3). The combined organic layers were washed with brine, dried over MgSO₄ and concentrated. The residue was purified by silica gel column chromatography (ethyl acetate/petrol ether 1/5-1/1) to afford (6-(4-chlorophenyl)-2-oxaspiro[3.5]non-6-en-7-yl)methanol (1.5 g, 78.9%) as white solid. ¹H NMR (400 MHz, CDCl₃) δ 7.34 (d, J=8.4 Hz, 2H), 7.07 (d, 2H, J=8.4 Hz), 4.54 (d, 2H, J=6.0 Hz), 4.46 (d, 2H, J=5.6 Hz), 3.93 (s, 2H), 2.62 (s, 2H), 2.40-2.33 (m, 2H), 2.03 (t, 2H, J=6.4 Hz).

### Intermediate 29

### Synthesis of 7-(Chloromethyl)-6-(4-chlorophenyl)-2-oxaspiro[3.5]non-6-ene

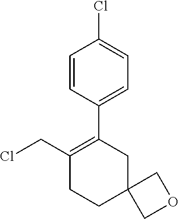 [SMILES: ClCC1=C(c2ccc(Cl)cc2)CC2(CC1)COC2]

To a solution of (6-(4-chlorophenyl)-2-oxaspiro[3.5]non-6-en-7-yl)methanol (1.5 g, 5.7 mmol) and triethylamine (836 mg, 8.6 mmol) in dichloromethane (15 ml) was added methylsulfonyl chloride (980 mg, 8.6 mmol) and the mixture was stirred at room temperature for 3.5 h. Solvent was removed under reduced pressure and the residue was purified by silica gel column chromatography to afford 7-(chloromethyl)-6-(4-chlorophenyl)-2-oxaspiro[3.5]non-6-ene (1.4 g, 87.0%) as white solid. ¹H NMR (400 MHz, CDCl₃) δ 7.35 (d, 2H, J=8.4 Hz), 7.16 (d, 2H, J=8.4 Hz), 4.53 (d, 2H, J=6.0 Hz), 4.45 (d, 2H, J=5.6 Hz), 3.86 (s, 2H), 2.64 (s, 2H), 2.40-2.33 (m, 2H), 2.03 (t, 2H, J=6.4 Hz).

### Intermediate 30

### Synthesis of Ethyl 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzoate

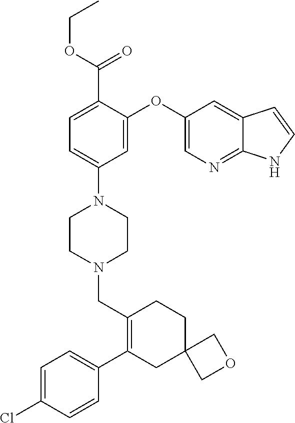 [SMILES: CCOC(=O)c1ccc(N2CCN(CC3=C(c4ccc(Cl)cc4)CC4(CC3)COC4)CC2)cc1Oc1cnc2[nH]ccc2c1]

To a solution of ethyl 2-((1H-pyrrolo[2,3-b]pyridin-5-yl)oxy)-4-(piperazin-1-yl)benzoate (382 mg, 0.82 mmol) in DMF (10 ml) were added 7-(chloromethyl)-6-(4-chlorophenyl)-2-oxaspiro[3.5]non-6-ene (200 mg, 0.75 mmol), potassium carbonate (310 mg, 2.25 mmol), DIPEA (290 mg, 2.25 mmol) and potassium iodide (24.9 mg, 0.15 mmol) and the mixture was stirred at room temperature overnight. The mixture was diluted with water and extracted with ethyl acetate (50 ml×3). The combined organic layers were washed with brine, concentrated and purified by silica gel column chromatography (ethyl acetate/petrol ether 1/5-1/1) to afford ethyl 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzoate (370 mg, 80.6%) as white solid. MS m/z 613 [M+H]⁺.

### Intermediate 31

### Synthesis of 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzoic acid

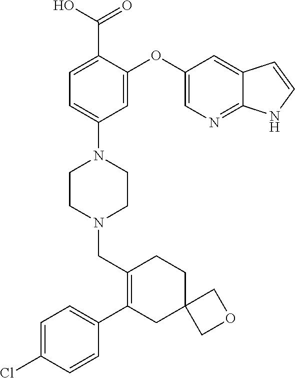 [SMILES: O=C(O)c1ccc(N2CCN(CC3=C(c4ccc(Cl)cc4)CC4(CC3)COC4)CC2)cc1Oc1cnc2[nH]ccc2c1]

To a solution of ethyl 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzoate (370 mg, 0.6 mmol) in dioxane (10 ml) was added 2N potassium hydroxide (6 ml, 12 mmol) and the mixture was stirred at 60° C. overnight. The solution was neutralized with 1N hydrochloric acid to pH 7 and extracted with ethyl acetate (100 ml×3). The combined organic layers were washed with brine, dried over magnesium sulfate and concentrated to afford 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzoic acid (1.1 g, 88.7%) as white solid. MS m/z 585 [M+H]⁺.

### Intermediate 32

### Synthesis of Methyl 2-((1H-pyrrolo[2,3-b]pyridin-5-yl)oxy)-4-(1-((6-(4-ch lorophenyl)spiro[3.5]non-6-en-7-yl) methyl)piperidin-4-yl)benzoate

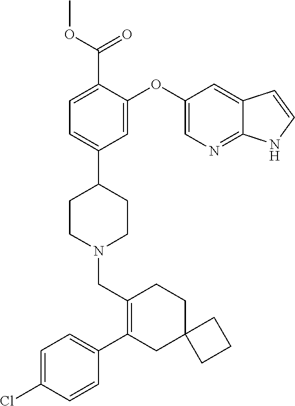 [SMILES: COC(=O)c1ccc(C2CCN(CC3=C(c4ccc(Cl)cc4)CC4(CCC4)CC3)CC2)cc1Oc1cnc2[nH]ccc2c1]

To as solution of 7-(chloromethyl)-6-(4-chlorophenyl)spiro[3.5]non-6-ene (850 mg, 3.04 mmol) in N,N-dimethyl formamide (10 ml) were added potassium carbonate (1.26 g, 2.2 mmol), potassium iodide (100 mg, 0.61 mmol), and methyl 2-((1H-pyrrolo[2,3-b]pyridin-5-yl)oxy)-4-(piperidin-4-yl)benzoate (1.0 g, 3.34 mmol) the mixture was stirred at room temperature overnight. Then the mixture was diluted with water and extracted with ethyl acetate. The combined organic layers were washed with brine and concentrated. The resulting residue was purified by silica gel column chromatography to afford methyl 2-((1H-pyrrolo[2,3-b]pyridin-5-yl)oxy)-4-(1-((6-(4-chlorophenyl)spiro[3.5]non-6-en-7-yl)methyl)piperidin-4-yl)benzoate (1.0 g, 55.2%) as light yellow solid. 1H NMR (400 MHz, CDCl₃) δ 9.39 (br s, 1H), 8.19 (d, J=2.6 Hz, 1H), 7.87 (d, J=8.1 Hz, 1H), 7.57 (d, J=2.6 Hz, 1H), 7.39 (dd, J=3.5, 2.5 Hz, 1H), 7.30-7.23 (m, 2H), 7.04-6.93 (m, 3H), 6.72 (d, J=1.6 Hz, 1H), 6.49 (dd, J=3.5, 2.0 Hz, 1H), 3.87 (s, 3H), 2.81-2.75 (m, 2H), 2.73-2.71 (m, 2H), 2.28 (s, 2H), 2.25-2.15 (m, 2H), 1.98-1.76 (m, 6H), 1.75-1.51 (m, 9H).

### Example 2—Synthesis of (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide (Compound 2)

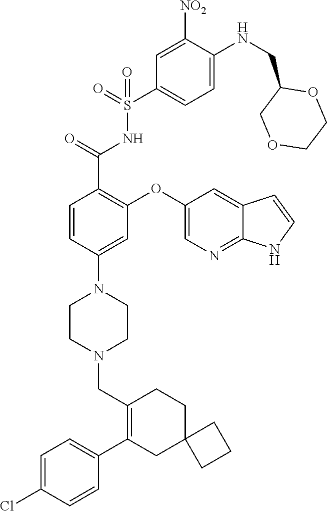 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

A mixture of 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoic acid, (R)-4-(((1,4-dioxan-2-yl)methyl)amino)-3-nitrobenzenesulfonamide, EDCI and 4-(N,N-dimethylamino)pyridine and dichloromethane was reacted at room temperature overnight, followed by the addition of water. The water layer was extracted with dichloromethane. The combined organic layers were washed with brine, concentrated and purified through silica gel chromatography to afford (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide. ¹H NMR (400 MHz, Methanol-d4) δ 8.66 (d, J=2.4 Hz, 1H), 7.99 (d, J=2.4 Hz, 1H), 7.84 (dd, J=9.2, 2.4 Hz, 1H), 7.64 (d, J=8.9 Hz, 1H), 7.51 (d, J=2.4 Hz, 2H), 7.45 (d, J=3.3 Hz, 1H), 7.37 (d, J=8.4 Hz, 2H), 7.10 (d, J=8.4 Hz, 2H), 6.94 (d, J=9.2 Hz, 1H), 6.76 (dd, J=8.9, 2.3 Hz, 1H), 6.40 (d, J=3.3 Hz, 1H), 6.36 (d, J=2.3 Hz, 1H), 3.87 (dd, J=11.8, 4.2 Hz, 3H), 3.83-3.70 (m, 3H), 3.67 (s, 2H), 3.62 (dd, J=11.7, 2.9 Hz, 1H), 3.51-3.41 (m, 2H), 3.40-3.35 (m, 1H), 3.29 (dq, J=3.2, 1.6 Hz, 1H), 2.41 (s, 2H), 2.26 (s, 2H), 2.00-1.77 (m, 6H).

### Example 3—Synthesis of (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide (Compound 1)

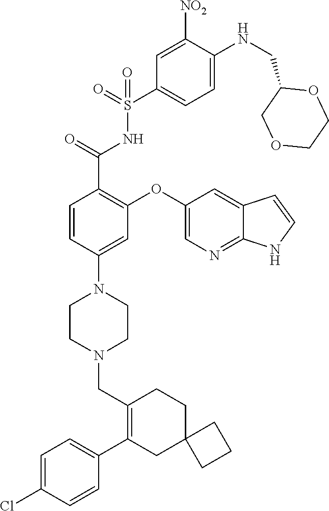 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

A mixture of 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoic acid, (S)-4-(((1,4-dioxan-2-yl)methyl)amino)-3-nitrobenzenesulfonamide, EDCI and 4-(N,N-dimethylamino)pyridine and dichloromethane was reacted at room temperature overnight, followed by the addition of water. The water layer was extracted with dichloromethane. The combined organic layers were washed with brine, concentrated and purified through silica gel chromatography to afford (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide. ¹H NMR (400 MHz, Methanol-d4) δ 8.66 (d, J=2.4 Hz, 1H), 7.99 (d, J=2.4 Hz, 1H), 7.84 (dd, J=9.2, 2.4 Hz, 1H), 7.64 (d, J=8.9 Hz, 1H), 7.51 (d, J=2.4 Hz, 2H), 7.45 (d, J=3.3 Hz, 1H), 7.37 (d, J=8.4 Hz, 2H), 7.10 (d, J=8.4 Hz, 2H), 6.94 (d, J=9.2 Hz, 1H), 6.76 (dd, J=8.9, 2.3 Hz, 1H), 6.40 (d, J=3.3 Hz, 1H), 6.36 (d, J=2.3 Hz, 1H), 3.87 (dd, J=11.8, 4.2 Hz, 3H), 3.83-3.70 (m, 3H), 3.67 (s, 2H), 3.62 (dd, J=11.7, 2.9 Hz, 1H), 3.51-3.41 (m, 2H), 3.40-3.35 (m, 1H), 3.29 (dq, J=3.2, 1.6 Hz, 1H), 2.41 (s, 2H), 2.26 (s, 2H), 2.00-1.77 (m, 6H).

### Example 4—Synthesis of (R)—N-((4-(((1,4-dioxan-2-yl)methyl)amino)-3-nitrophenyl)sulfonyl)-2-((1H-pyrrolo[2,3-b]pyridin-5-yl)oxy)-4-(1-((6-(4-chlorophenyl)spiro[3.5]non-6-en-7-yl)methyl)-1,2,3,6-tetrahydropyridin-4-yl)benzamide

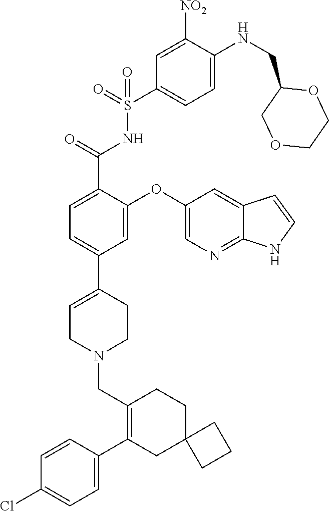 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CCC4)CC3)CC2)cc1Oc1cnc2[nH]ccc2c1]

To a solution of (R)—N-((4-(((1,4-dioxan-2-yl)methyl)amino)-3-nitrophenyl)sulfonyl)-2-((1H-pyrrolo[2,3-b]pyridin-5-yl)oxy)-4-bromobenzamide in 1,2-dimethoxy-ethane (10 ml) and water (1 ml) were added 1-((6-(4-chlorophenyl)spiro[3.5]non-6-en-7-yl)methyl)-4-(4,4,5,5-tetramethyl-1,3,2-dioxaborolan-2-yl)-1,2,3,6-tetrahydropyridine, Pd(dppf)Cl₂, and K₂CO₃, and the mixture was stirred at 80° C. for 12 h. The reaction was cooled to room temperature and diluted with water. The mixture was extracted with ethyl acetate (30 ml×3), dried over anhydrous MgSO₄, and concentrated. The residue was purified by C₁₈ reversed phase preparative HPLC to give (R)—N-((4-(((1,4-dioxan-2-yl)methyl)amino)-3-nitrophenyl)sulfonyl)-2-((1H-pyrrolo[2,3-b]pyridin-5-yl)oxy)-4-(1-((6-(4-chlorophenyl)spiro[3.5]non-6-en-7-yl)methyl)-1,2,3,6-tetrahydropyridin-4-yl)benzamide. ¹H NMR (400 MHz, Methanol-d₄) δ 8.68 (d, J=2.3 Hz, 1H), 7.97 (d, J=2.6 Hz, 1H), 7.88 (dd, J=9.3, 2.3 Hz, 1H), 7.63 (d, J=8.2 Hz, 1H), 7.50 (d, J=2.6 Hz, 1H), 7.46 (d, J=3.5 Hz, 1H), 7.30 (d, J=8.4 Hz, 2H), 7.16 (dd, J=8.2, 1.7 Hz, 1H), 7.10 (d, J=8.4 Hz, 2H), 6.94 (d, J=9.3 Hz, 1H), 6.85 (d, J=1.7 Hz, 1H), 6.41 (d, J=3.5 Hz, 1H), 5.94-5.90 (m, 1H), 3.95-3.40 (m, 14H), 3.15-3.03 (m, 1H), 2.68-2.45 (m, 2H), 2.43 (s, 2H), 2.30-2.20 (m, 2H), 2.03-1.77 (m, 8H).

### Example 5—Synthesis of 2-((1H-pyrrolo[2,3-b]pyridin-5-yl)oxy)-4-(1-((6-(4-chlorophenyl)-2-oxaspiro[3.5]non-6-en-7-yl)methyl)-1,2,3,6-tetrahydropyridin-4-yl)-N-((3-nitro-4-(((tetrahydro-2H-pyran-4-yl)methyl)amino)phenyl)sulfonyl)benzamide

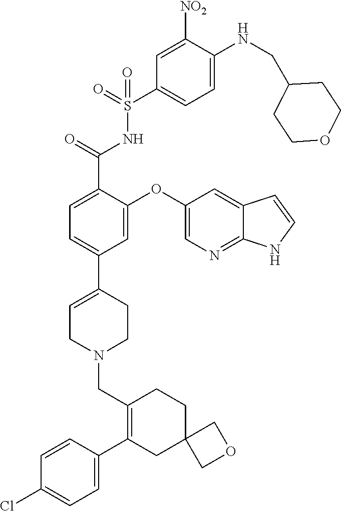 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CC3)COC4)CC2)cc1Oc1cnc2[nH]ccc2c1]

The title compound was prepared using a procedure similar to the one described for EXAMPLE 4. ¹H NMR (400 MHz, Methanol-d₄) δ 8.70 (d, J=2.3 Hz, 1H), 7.99 (d, J=2.5 Hz, 1H), 7.90 (dd, J=9.2, 2.3 Hz, 1H), 7.61 (d, J=8.2 Hz, 1H), 7.57 (d, J=2.5 Hz, 1H), 7.48 (d, J=3.5 Hz, 1H), 7.31 (d, J=8.4 Hz, 2H), 7.20-7.10 (m, 3H), 6.96 (d, J=9.2 Hz, 1H), 6.82 (d, J=1.6 Hz, 1H), 6.44 (d, J=3.5 Hz, 1H), 5.93-5.86 (m, 1H), 4.53 (d, J=5.9 Hz, 2H), 4.49 (d, J=5.9 Hz, 2H), 4.00-3.90 (m, 2H), 3.77-3.33 (m, 7H), 3.26 (d, J=7.0 Hz, 2H), 3.15-3.00 (m, 1H), 2.70-2.65 (m, 2H), 2.63-2.25 (m, 4H), 2.07 (t, J=6.3 Hz, 2H), 2.00-1.85 (m, 1H), 1.75-1.65 (m, 2H), 1.46-1.30 (m, 2H).

### Example 6—Synthesis of 2-((1H-pyrrolo[2,3-b]pyridin-5-yl)oxy)-4-(1-((6-(4-chlorophenyl)spiro[3.5]non-6-en-7-yl)methyl)-1,2,3,6-tetrahydropyridin-4-yl)-N-((3-nitro-4-(((tetrahydro-2H-pyran-4-yl)methyl)amino)phenyl)sulfonyl)benzamide

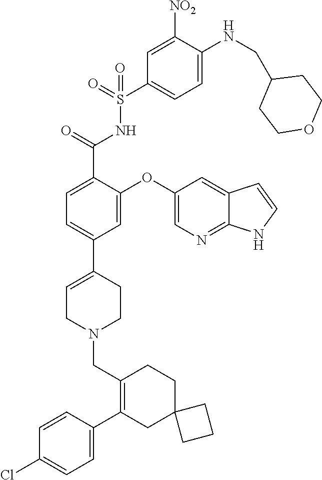 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CCC4)CC3)CC2)cc1Oc1cnc2[nH]ccc2c1]

The title compound was prepared using a procedure similar to the one described for EXAMPLE 4. ¹H NMR (400 MHz, Methanol-d₄) δ 8.71 (t, J=1.9 Hz, 1H), 8.00-7.95 (m, 1H), 7.90 (dd, J 9.3, 1.9 Hz, 1H), 7.63 (dd, J=8.1, 1.4 Hz, 1H), 7.56-7.50 (m, 1H), 7.46 (dd, J=3.5, 1.4 Hz, 1H), 7.33-7.26 (m, 2H), 7.18-7.06 (m, 3H), 6.96 (dd, J=9.3, 1.4 Hz, 1H), 6.81 (s, 1H), 6.43 (dd, J=3.5, 1.5 Hz, 1H), 5.93-5.86 (m, 1H), 4.00-3.94 (m, 2H), 3.83-3.36 (m, 7H), 3.26 (d, J=7.0 Hz, 2H), 3.10-3.04 (m, 1H), 2.67-2.40 (m, 4H), 2.30-2.24 (m, 2H), 2.02-1.77 (m, 9H), 1.74-1.67 (m, 2H), 1.45-1.30 (m, 2H).

### Example 7—Synthesis of (R)—N-((4-(((1,4-dioxan-2-yl)methyl)amino)-3-nitrophenyl)sulfonyl)-2-((1H-pyrrolo[2,3-b]pyridin-5-yl)oxy)-4-(1-((6-(4-chlorophenyl)-2-oxaspiro[3.5]non-6-en-7-yl)methyl)-1,2,3,6-tetrahydropyridin-4-yl)benzamide

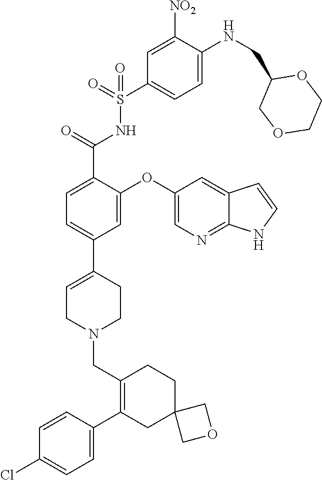 [SMILES: O=C(NS(=O)(=O)c1ccc(NC[C@@H]2COCCO2)c([N+](=O)[O-])c1)c1ccc(C2=CCN(CC3=C(c4ccc(Cl)cc4)CC4(CC3)COC4)CC2)cc1Oc1cnc2[nH]ccc2c1]

The title compound was prepared using a procedure similar to the one described for EXAMPLE 4. ¹H NMR (400 MHz, Methanol-d₄) δ 8.68 (d, J=2.3 Hz, 1H), 7.99 (d, J=2.5 Hz, 1H), 7.89 (dd, J=9.2, 2.3 Hz, 1H), 7.65 (d, J=8.2 Hz, 1H), 7.54 (d, J=2.5 Hz, 1H), 7.48 (d, J=3.4 Hz, 1H), 7.33 (d, J=8.4 Hz, 2H), 7.21-7.16 (m, 1H), 7.13 (d, J=8.4 Hz, 2H), 6.95 (d, J=9.3 Hz, 1H), 6.86 (d, J=1.6 Hz, 1H), 6.43 (d, J=3.5 Hz, 1H), 5.94-5.90 (m, 1H), 4.60-4.43 (m, 4H), 3.95-3.40 (m, 14H), 3.15-3.00 (m, 1H), 2.80-2.60 (m, 4H), 2.38-2.25 (m, 2H), 2.08 (t, J=6.3 Hz, 2H).

### Example 8—Synthesis of (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzamide

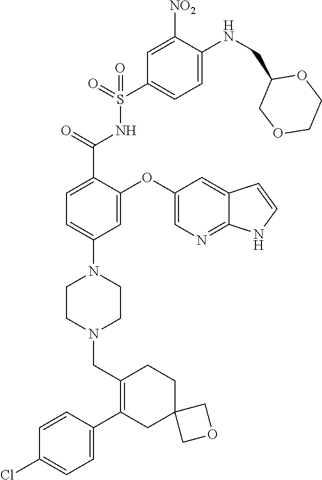 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CC3)COC4)CC2)cc1Oc1cnc2[nH]ccc2c1]

A mixture of 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzoic acid (290 mg, 0.5 mmol), (R)-4-(((1,4-dioxan-2-yl)methyl)amino)-3-nitrobenzenesulfonamide (236 mg, 0.75 mmol), EDCI (191 mg, 1 mmol), 4-(N,N-dimethylamino)pyridine (591 mg, 0.75 mmol) in dichloromethane (15 ml) was stirred at room temperature overnight. The solvent was removed under vacuum and the resulting residue was purified through a silica gel column to afford (R)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzamide (150 mg, 34.1%) as yellow solid. ¹H NMR (400 MHz, Methanol-d₄) δ 8.67 (d, J=2.3 Hz, 1H), 7.99 (d, J=2.3 Hz, 1H), 7.85 (dd, J=9.3, 2.3 Hz, 1H), 7.64 (d, J=8.8 Hz, 1H), 7.52 (d, J=2.3 Hz, 1H), 7.45 (d, J=3.5 Hz, 1H), 7.39 (d, J=8.4 Hz, 2H), 7.13 (d, J=8.4 Hz, 2H), 6.95 (d, J=9.3 Hz, 1H), 6.76 (dd, J=8.8, 2.4 Hz, 1H), 6.41 (d, J=3.5 Hz, 1H), 6.34 (d, J=2.4 Hz, 1H), 4.54 (d, J=5.9 Hz, 2H), 4.48 (d, J=5.9 Hz, 2H), 3.93-3.35 (m, 19H), 2.70-2.65 (m, 2H), 2.33 (s, 2H), 2.08 (t, J=6.3 Hz, 2H).

### Example 9—Synthesis of 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)-N-((3-nitro-4-(((tetrahydro-2H-pyran-4-yl)methyl)amino)phenyl)sulfonyl)benzamide

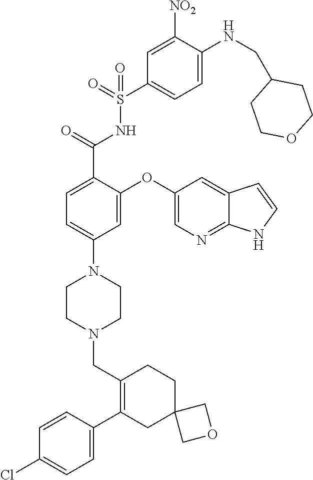 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1]

A mixture of 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)benzoic acid (250 mg, 0.43 mmol), 3-nitro-4-(((tetrahydro-2H-pyran-4-yl)methyl)amino)benzenesulfonamide (202 mg, 0.64 mmol), EDCI (164 mg, 0.86 mmol), 4-(N,N-dimethylamino)pyridine (78 mg, 0.64 mmol) in dichloromethane (10 ml) was stirred at room temperature overnight, followed by concentration. The resulting residue was purified through silica gel chromatography to afford 2-((1H-pyrrolo[2,3-b]pyridin-5-yl)oxy)-4-(4-((6-(4-chlorophenyl)-2-oxaspiro[3.5]non-6-en-7-yl)methyl)piperazin-1-yl)-N-((3-nitro-4-(((tetrahydro-2H-pyran-4-yl)methyl)amino)phenyl)sulfonyl)benzamide (150 mg, 39.6%) as a yellow solid. ¹H NMR (400 MHz, Methanol-d₄) δ 8.70 (d, J=2.3 Hz, 1H), 8.01 (d, J=2.6 Hz, 1H), 7.87 (dd, J=9.2, 2.3 Hz, 1H), 7.66 (d, J=8.8 Hz, 1H), 7.56 (d, J=2.6 Hz, 1H), 7.47 (d, J=3.5 Hz, 1H), 7.39 (d, J=8.4 Hz, 2H), 7.13 (d, J=8.4 Hz, 2H), 6.97 (d, J=9.2 Hz, 1H), 6.76 (dd, J=8.8, 2.4 Hz, 1H), 6.43 (d, J=3.5 Hz, 1H), 6.32 (d, J=2.4 Hz, 1H), 4.54 (d, J=5.9 Hz, 2H), 4.48 (d, J=5.9 Hz, 2H), 4.03-3.94 (m, 2H), 3.67 (s, 2H), 3.55-3.27 (m, 12H), 2.69 (s, 2H), 2.35-2.25 (m, 2H), 2.08 (t, J=6.3 Hz, 2H), 2.05-1.93 (m, 1H), 1.76-1.69 (m, 2H), 1.45-1.35 (m, 2H).

### Example 10—Synthesis of 2-((1H-pyrrolo[2,3-b]pyridin-5-yl)oxy)-4-(4-((6-(4-chlorophenyl)spiro[3.5]non-6-en-7-yl)methyl)piperazin-1-yl)-N-((3-nitro-4-(((tetrahydro-2H-pyran-4-yl)methyl)amino)phenyl)sulfonyl)benzamide

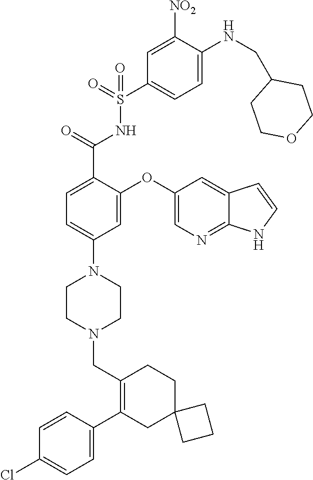 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1]

A mixture of 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoic acid (1.75 g, 3 mmol), 3-nitro-4-(((tetrahydro-2H-pyran-4-yl)methyl)amino)benzenesulfonamide (1.43 g, 4.5 mmol), EDCI (1.15 g, 6 mmol) and 4-(N,N-dimethylamino)pyridine (550 mg, 4.5 mmol) and dichloromethane (40 ml) was reacted at room temperature overnight, followed by the addition of water. The water layer was extracted with dichloromethane. The combined organic layers were washed with brine, concentrated and purified through silica gel column to afford 2-((1H-pyrrolo[2,3-b]pyridin-5-yl)oxy)-4-(4-((6-(4-chlorophenyl)spiro[3.5]non-6-en-7-yl) methyl)piperazin-1-yl)-N-((3-nitro-4-(((tetrahydro-2H-pyran-4-yl)methyl)amino)phenyl)sulfonyl)benzamide (1.7 g, 64.4%) as a yellow solid. 1H NMR (400 MHz, Methanol-d₄) δ 8.70 (d, J=2.3 Hz, 1H), 8.01 (d, J=2.7 Hz, 1H), 7.87 (d, J=9.2, 2.3 Hz, 1H), 7.66 (d, J=8.9 Hz, 1H), 7.55 (d, J=2.7 Hz, 1H), 7.47 (d, J=3.4 Hz, 1H), 7.38 (d, J=8.4 Hz, 2H), 7.10 (d, J=8.4 Hz, 2H), 6.97 (d, J=9.2 Hz, 1H), 6.77 (dd, J=8.9, 2.4 Hz, 1H), 6.44 (d, J=3.4 Hz, 1H), 6.34 (d, J=2.4 Hz, 1H), 4.02-3.94 (m, 3H), 3.66 (s, 3H), 3.49-3.38 (m, 2H), 3.41-3.25 (m, 7H), 2.42 (s, 3H), 2.26 (s, 3H), 2.00-1.67 (m, 4H), 1.45-1.38 (m, 2H).

### Example 11—Synthesis of 2-((1H-pyrrolo[2,3-b]pyridin-5-yl)oxy)-4-(4-((6-(4-chlorophenyl) spiro[3.5]non-6-en-7-yl)methyl)piperazin-1-yl)-N-((3-nitrophenyl)sulfonyl)benzamide

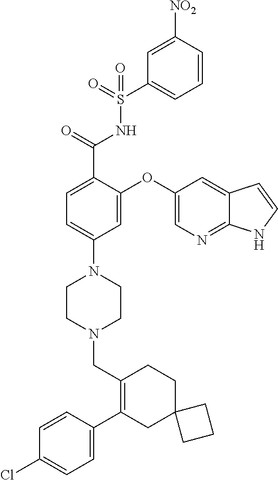 [SMILES: O=C(NS(=O)(=O)c1cccc([N+](=O)[O-])c1)c1ccc(N2CCN(CC3=C(c4ccc(Cl)cc4)CC4(CCC4)CC3)CC2)cc1Oc1cnc2[nH]ccc2c1]

The title compound was prepared using a procedure similar to the one described for EXAMPLE 10. ¹H NMR (400 MHz, DMSO-d₆) δ 11.70 (s, 1H), 9.47 (s, 1H), 8.62 (d, J=2.2 Hz, 1H), 8.44 (d, J=8.3 Hz, 1H), 8.27 (d, J=7.9 Hz, 1H), 8.02-7.97 (m, 1H), 7.84-7.75 (m, 1H), 7.56-7.43 (m, 3H), 7.40 (d, J=8.3 Hz, 2H), 7.11 (d, J=8.3 Hz, 2H), 6.72 (d, J=8.9 Hz, 1H), 6.40-6.35 (m, 1H), 6.30 (s, 1H), 3.80-3.65 (m, 2H), 3.55 (s, 2H), 3.28-2.95 (m, 4H), 2.82-2.65 (m, 2H), 2.31 (s, 2H), 2.22-2.15 (m, 2H), 1.93-1.60 (m, 8H).

### Example 12—Synthesis of 2-((1H-pyrrolo[2,3-b]pyridin-5-yl)oxy)-4-(4-((6-(4-chlorophenyl) spiro[3.5]non-6-en-7-yl)methyl)piperazin-1-yl)-N-((4-(methylamino)-3-nitrophenyl)sulfonyl)benzamide

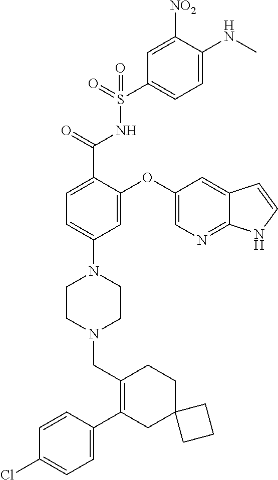 [SMILES: CNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]]

The title compound was prepared using a procedure similar to the one described for EXAMPLE 10. ¹H NMR (400 MHz, Methanol-d₄) δ 8.78 (d, J=2.3 Hz, 1H), 8.05 (d, J=2.6 Hz, 1H), 7.96 (dd, J=9.2, 2.3 Hz, 1H), 7.80 (d, J=8.9 Hz, 1H), 7.61 (d, J=2.6 Hz, 1H), 7.46 (d, J=3.5 Hz, 1H), 7.34 (d, J=8.4 Hz, 2H), 7.01 (d, J=8.4 Hz, 2H), 6.90 (d, J=9.2 Hz, 1H), 6.68 (dd, J=8.9, 2.4 Hz, 1H), 6.46 (d, J=3.5 Hz, 1H), 6.18 (d, J=2.4 Hz, 1H), 3.60 (s, 2H), 3.50-3.12 (m, 8H), 3.06 (s, 3H), 2.38 (s, 2H), 2.30-2.16 (m, 2H), 1.97-1.73 (m, 8H).

### Example 13-Synthesis of 2-((1H-pyrrolo[2,3-b]pyridin-5-yl)oxy)-4-(4-((6-(4-chlorophenyl) spiro[3.5]non-6-en-7-yl)methyl)piperazin-1-yl)-N-((4-(dimethylamino)-3-nitrophenyl) sulfonyl)benzamide

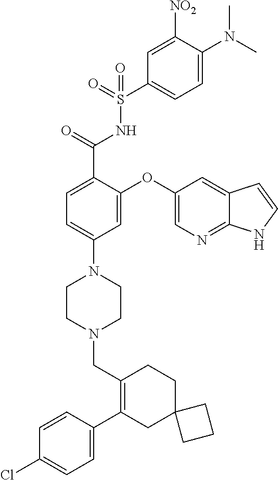 [SMILES: CN(C)c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]]

The title compound was prepared using a procedure similar to the one described for EXAMPLE 10. ¹H NMR (400 MHz, Methanol-d₄) δ 8.41 (d, J=2.2 Hz, 1H), 8.08 (d, J=2.5 Hz, 1H), 7.91 (dd, J=9.4, 2.3 Hz, 1H), 7.81 (d, J=8.9 Hz, 1H), 7.68 (d, J=2.3 Hz, 1H), 7.48 (d, J=3.5 Hz, 1H), 7.34 (d, J=8.0 Hz, 2H), 7.04 (d, J=9.4 Hz, 1H), 7.01 (d, J=8.0 Hz, 2H), 6.71-6.63 (m, 1H), 6.51 (d, J 3.5 Hz, 1H), 6.15 (d, J=1.9 Hz, 1H), 3.59 (s, 2H), 3.52-3.20 (m, 8H), 2.98 (s, 6H), 2.38 (s, 2H), 2.25-2.17 (m, 2H), 1.96-1.72 (m, 8H).

### Example 14—Synthesis of 2-((1H-pyrrolo[2,3-b]pyridin-5-yl)oxy)-4-(1-((6-(4-chlorophenyl)spiro[3.5]non-6-en-7-yl)methyl)piperidin-4-yl)-N-((3-nitro-4-(((tetrahydro-2H-pyran-4-yl)methyl)amino)phenyl)sulfonyl)benzamide

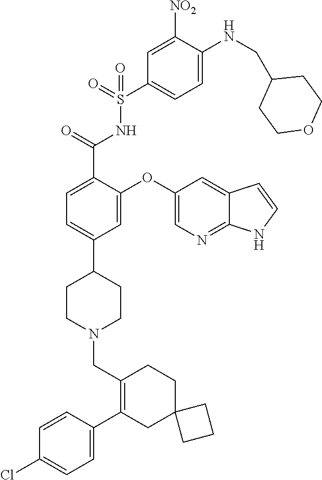 [SMILES: O=C(NS(=O)(=O)c1ccc(NCC2CCOCC2)c([N+](=O)[O-])c1)c1ccc(C2CCN(CC3=C(c4ccc(Cl)cc4)CC4(CCC4)CC3)CC2)cc1Oc1cnc2[nH]ccc2c1]

A mixture of 2-((1H-pyrrolo[2,3-b]pyridin-5-yl)oxy)-4-(1-((6-(4-chlorophenyl)spiro[3.5]non-6-en-7-yl)methyl)piperidin-4-yl)benzoic acid (200 mg, 0.34 mmol), 3-nitro-4-(((tetrahydro-2H-pyran-4-yl)methyl)amino)benzenesulfonamide (162 mg, 0.52 mmol), EDCI (130 mg, 0.68 mmol), 4-(N,N-dimethylamino)pyridine (63.4 mg, 0.52 mmol) in dichloromethane (15 ml) was stirred at room temperature overnight, followed by purification by silica gel column chromatography to afford 2-((1H-pyrrolo[2,3-b]pyridin-5-yl)oxy)-4-(1-((6-(4-chlorophenyl)spiro[3.5]non-6-en-7-yl)methyl)piperidin-4-yl)-N-((3-nitro-4-(((tetrahydro-2H-pyran-4-yl) methyl)amino)phenyl)sulfonyl)benzamide (170 mg, 57.3%) as a yellow solid. ¹H NMR (400 MHz, DMSO-d₆) δ 11.94 (s, 1H), 11.64 (s, 1H), 8.50-8.42 (m, 2H), 7.97 (d, J=2.6 Hz, 1H), 7.76 (dd, J=9.2, 2.2 Hz, 1H), 7.52-7.36 (m, 5H), 7.11 (d, J=7.9 Hz, 2H), 6.99 (d, J 9.2 Hz, 1H), 6.91-6.86 (m, 1H), 6.55 (s, 1H), 6.37 (s, 1H), 3.89-3.79 (m, 2H), 3.35-2.90 (m, 10H), 2.32-2.10 (m, 5H), 1.95-1.15 (m, 17H).

### Example 15—Compounds of Formula (I) Inhibit Bcl-2 and Bcl-xL

Fluorescein labeled BIM (81-106), BAK (72-87), and BID (79-99) peptides, named as Flu-BIM, Flu-BAK, and Flu-BID, respectively, were used as the fluorescent probes in FP assays for Bcl-2, Bcl-xL, and Mcl-1, respectively. By monitoring the total fluorescence polarization values of mixtures composed of fluorescent probes at fixed concentrations and proteins with increasing concentrations up to the full saturation, the K_{d} values of Flu-BIM to Bcl-2, Flu-BAK to Bcl-xL and Flu-BID to Mcl-1 were determined to be 0.55±0.15 nM, 4.4±0.8 nM, and 6.9±0.9 nM, respectively. Fluorescence polarization values were measured using an Infinite M-1000 plate reader (Tecan U.S., Research Triangle Park, N.C.) in Microfluor 96-well, black, round-bottom plates (Thermo Scientific). To each well, 1 nM of Flu-BIM, or 2 nM of Flu-BAK or 2 nM of Flu-BID and increasing concentrations of Bcl-2, or Bcl-xL, or Mcl-1 were added to a final volume of 125 μL in the assay buffer (100 mM potassium phosphate, pH 7.5, 100 μg/mL bovine γ-globulin, 0.02% sodium azide, Invitrogen, with 0.01% Triton X-100 and 4% DMSO). Plates were mixed and incubated at room temperature for 1 hour with gentle shaking to assure equilibrium. The polarization values in millipolarization units (mP) were measured at an excitation wavelength of 485 nm and an emission wavelength of 530 nm. Equilibrium dissociation constants (K_{d}) were then calculated by fitting the sigmoidal dose-dependent FP increases as a function of protein concentrations using Graphpad Prism 5.0 software (Graphpad Software, San Diego, Calif.).

Kᵢ values of representative compounds of the disclosure to Bcl-2, Bcl-xL, and Mcl-1 were determined from competitive binding experiments in which serial dilutions of inhibitors were added into 96-well plates containing fixed concentration of the fluorescent probes and proteins in each well. Mixtures of 5 μL of the tested inhibitors in DMSO and 120 μL of pre-incubated protein/probe complexes in the assay buffer were added into assay plates and incubated at room temperature for 2 hours with gentle shaking. Final concentrations of the protein and probe are 1.5 nM and 1 nM for the Bcl-2 assay, 10 nM and 2 nM for the Bcl-xL assay, and 20 nM and 2 nM for Mcl-1 assay, respectively. Negative controls containing protein/probe complex only (equivalent to 0% inhibition), and positive controls containing free probe only (equivalent to 100% inhibition), were included in each assay plate. Fluorescence polarization values were measured as described above. IC₅₀ values were determined by nonlinear regression fitting of the competition curves. The Kᵢ values of competitive inhibitors were calculated using an equation described in Nikolovska-Coleska et al., Analytical Biochemistry 332:261-73 (2004), based upon the measured IC₅₀ values, the values of the probes to the proteins, and the concentrations of the proteins and probes in the competitive assays. K_{d} values were also calculated using the equation of Huang, Journal of Biomolecular Screening 8:34-38 (2003).

The inhibitory activities of representative Compounds of the Disclosure against Bcl-2, Bcl-xL, and Mcl-1 are provided in Table 2.

TABLE 2

| | Inhibitory Activity | | |
|---|---|---|---|
| | IC₅₀ (nM) | | |
| Cpd. No. | Bcl-2 | Bcl-xL | Mcl-1 |
| 1 | 2.0 | 15.7 | >5000 |
| 2 | 1.3 | 14.8 | |
| 3 | 1.4 | 9.2 | |
| 4 | 0.76 | 10.6 | |
| 5 | 1.2 | 13.7 | |
| 6 | 3.1 | 8.6 | |
| 7 | 2.1 | 14 | |
| 8 | 2.4 | 15.7 | |
| 9 | 2.4 | 6.4 | >5000 |
| 10 | 1.9 | 20.6 | |
| 11 | 3.3 | 14.0 | |
| 12 | 11.9 | 77.8 | |
| 13 | 4.4 | 139 | |
| 14 | 3.8 | 19.2 | |
| 15 | 5.0 | 20.7 | |
| 16 | 2.1 | 67.5 | |
| 17 | 2.1 | 13.1 | |
| 18 | 1.3 | 7.1 | |
| 19 | 1.4 | 9.9 | |
| 20 | 2.7 | 12.0 | |

### Example 16—Treatment with Compound 1 Improves NAS Score in a HFD/CCl₄ NASH Mouse Model

The ability of Compound 1 to treat non-alcoholic steatohepatitis in vivo was demonstrated using a mouse model of NASH.

Male C57BL/6J mice with an age of 18 weeks were purchased from GemPharmatech Co., Ltd (Nanjing, Jiangsu). Mice were first fed with high-fat diet (HFD) for 11 weeks to induced obesity. Carbon tetrachloride (25% CCl₄, 0.5 μl/g) was then intraperitoneally injected twice weekly for 4 consecutive weeks, to induce liver damages, leading to inflammation, cell death and fibrosis. The day of the first carbon tetrachloride injection was dated as day 0. At day 7, the animals were randomized and treatment started. The test article administration and the animal numbers in each group are shown in the following experimental design Table 3.

TABLE 3

| | | | Dose | | | |
|---|---|---|---|---|---|---|
| | Animal | | level | Dose | Dosing | Dosing |
| Group | No. | Treatment | (mg/kg) | Route | Frequency | Duration |
| 1 | 5 | Vehicle | — | i.v. | biw | Day 7-27 |
| 2 | 8 | Vehicle | — | i.v. | biw | Day 7-27 |
| 3 | 8 | Compound 1 | 50 | p.o. | q.d. | Day 7-27 |
| 4 | 8 | OCA | 30 | p.o. | q.d. | Day 7-27 |
| 5 | 8 | OCA + | 30 + 50 | p.o. | q.d. | Day 7-27 |
| | | Compound 1 | | | | |

Note:
Group 1 were fed a normal diet and intraperitoneally injected with olive oil in place of CCl₄.
Vehicle: 10% PEG400, 5% Cremophor EL, 0.1N NaOH, 1x phosphate buffered saline

All animals were euthanatized at day 28 to collect blood and liver samples for biochemical and histological analysis.

Liver tissue from animals in each group were analyzed for NAS scores.

Specifically, the scores assessed were:
1) the unweighted sum of the steatosis (0-3);
2) lobular inflammation (0-3);
3) hepatocellular ballooning (0-2); and
4) liver fibrosis score (0-4), after Sirius red staining.

Liver tissues of each group of animals were taken, and NAS scores were analyzed by hematoxylin-eosin (HE) staining, and liver fibrosis was analyzed by Sirius red staining.

After staining, the NAS scores of the prepared samples were evaluated. In hematoxylin-eosin stained sections, NAS scores were evaluated according to the degree of steatosis, inflammatory cell infiltration and steatosis of liver cells. Five visual field comprehensive scores are selected for each liver lobe, and the evaluation criteria are presented in Table 4.

TABLE 4
Non-alcoholic hepatic steatohepatitis (NASH)

| Pathological findings | Evaluation criteria | Score |
|---|---|---|
| Ballooning of liver cells | None | 0 |
| | Slight cell swelling | 1 |
| | Massive cell swelling | 2 |
| Intralobular inflammation | None | 0 |
| | <2 inflammatory foci/200 × | 1 |
| | visual field | |
| | 2-4 inflammatory foci/200 × | 2 |
| | visual field | |
| | >4 inflammatory foci/200 × | 3 |
| | visual field | |
| Hepatocyte steatosis: the | <5% | 0 |
| area occupied in the entire | 5%-33% | 1 |
| section | >33%-66% | 2 |
| | >66% | 3 |

| Pathological diagnosis | NAS score |
|---|---|
| NASH disease | >5 |
| Suspected NASH | 3-4 |
| Non-NASH disease | <2 |

Notes:
a) Ballooning is characterized by the hepatocytes swelling and the cytoplasm becoming loose; the hepatocytes adopt a spherical shape, and the cytoplasm is almost transparent;
b) Steatosis is characterized by vacuole-like regular round lipid droplets in the liver cells, which are divided into large lipid droplets and small lipid droplets. Large lipid droplets can push the liver cell nucleus to the edge of the cell, and small lipid droplets can accumulate in the liver, the nucleus is located in the center of the cell;
c) In the portal area of the liver lobules, inflammatory cell infiltration is characterized by the hepatocyte cord and the central venous area being scattered or by the presence of a large number of inflammatory cells, mainly neutrophils and macrophages.
d) Watery degeneration of liver cells is characterized by irregular vacuoles of varying sizes in the liver cells, and the cytoplasm is a network of varying degrees.

After staining, fibrosis of the liver was additionally evaluated. All sections stained with Sirius Red were scanned by an Aperio VERSA 200 Brightfield & Fluorescenc slice scanner. Two fields were randomly selected under a 5×field of view. The two fields covered 85% of the liver tissue area. Quantitative analysis to calculate the area of fibrosis deposition in the slices was performed.

Prism, version 6, (GraphPad Software Inc., San Diego, CA) was used for all statistical analysis and for graphic presentation.

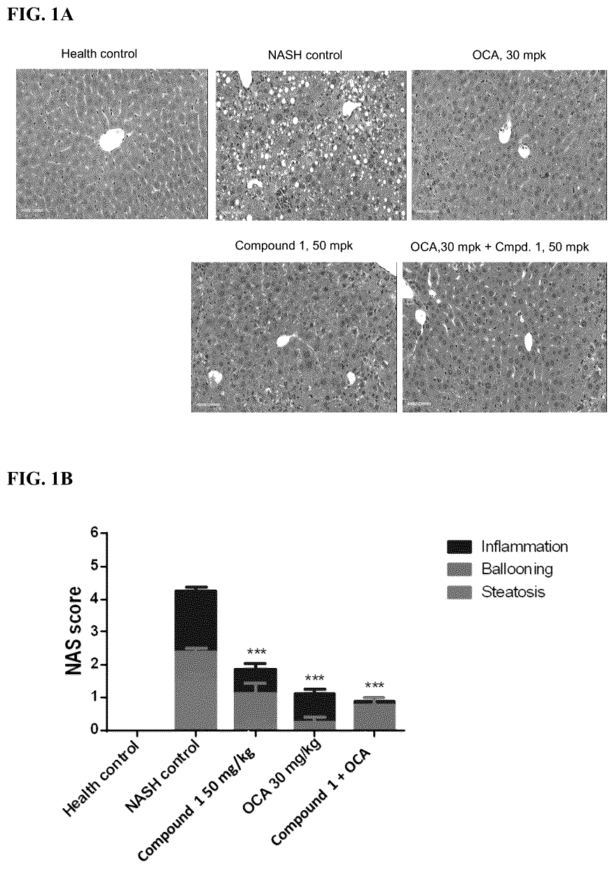

To study the effect of Compound 1 as single agent, or in combination with the FXR agonist obeticholic acid (OCA), on NASH and fibrosis, C57BL/6J mice induced with high-fat diet (HFD) and CCl₄ were treated with Compound 1 at 50 mg/kg (qd, p.o.), and OCA at 30 mg/kg (qd, p.o.) as a single agent, or in combinations as detailed in Table 3. After drug treatment for 21 days, animals were euthanatized to collect liver samples for pathological analysis. HE-staining of liver tissue demonstrated that compared to the healthy control, high-fat diet and CCl₄ injection induced severe steatosis, lobular inflammation and hepatocellular ballooning, all of which were alleviated after treatment with Compound 1 and OCA, alone or in combination (FIG. 1A).

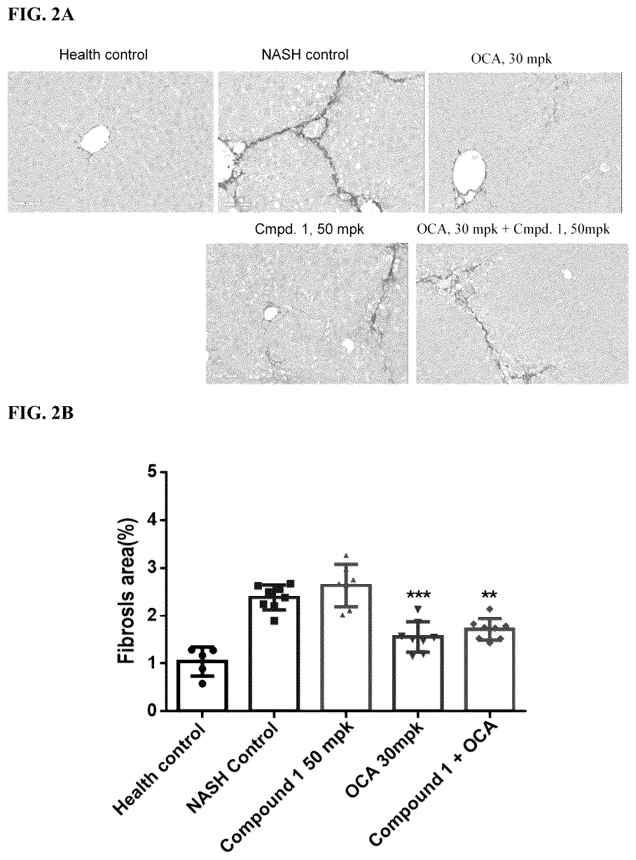

As shown in the NAS score analysis (FIG. 1B), Compound 1 reduced inflammation and steatosis, with modest effects on ballooning. Compound 1 demonstrated a better anti-inflammation effect than OCA alone. Compound 1 and OCA in combination showed better activity on NAS score than Compound 1 or OCA as a single agent. Notably, significant reduction of inflammation was observed after combination treatment with Compound 1 and OCA, in comparison to single agent treatment. Compound 1 showed modest effect on fibrosis as a single agent, in comparison to OCA, as shown by Sirius red staining of liver tissues (FIG. 2A) and fibrosis score analysis (FIG. 2B).

Collectively, these results suggest that Compound 1 is efficacious for the treatment of non-alcoholic steatohepatitis, based on NAS score alleviation through reduced inflammation, ballooning and steatosis in a mouse model of non-alcoholic steatosis. Additionally, Compound 1 has demonstrated potential to alleviate fibrosis in this non-alcoholic steatohepatitis model, underscoring that Compound 1 is useful for the treatment of non-alcoholic steatohepatitis.

### Example 17—Treatment with Compound 1 Improves NAS Score in a HFD/CCl₄ NASH Mouse Model

Male SD rats, with an age of 10 days, were purchased from Vital River Co., Ltd (Beijing).

The rats were given a one-time intraperitoneal injection of diethylnitrosamine (DEN) two weeks after birth, and continued breast milk feeding for two weeks.

After four weeks, a total of 56 male rats were selected and divided into 7 groups according to animal weight. The rats were started on a high-fat, high-cholesterol diet (HFD-CHOL), with a feeding cycle of 12 weeks. Before intraperitoneal injection of DEN, 5 male rats were randomly selected for parallel feeding as a control cohort, which were fed a standard diet and not injected with DEN.

After 7 weeks of HFD-CHOL diet feeding, the animals were randomized and treatment with vehicle or the indicated compound was started. The test article administration and the animal numbers in each group are shown in the following experimental design Table 5.

TABLE 5

| | Animal | HFD-CHOL | | Dose level | Dose | Dosing | Dosing |
|---|---|---|---|---|---|---|---|
| Group | No. | diet | Treatment | (mg/kg) | Route | Frequency | Duration |
| 1 | 5 | No | Vehicle | — | p.o. | q.d. | 42 days |
| 2 | 8 | Yes | Vehicle | — | p.o. | q.d. | 42 days |
| 3 | 8 | Yse | GFT505 | 30 | p.o. | q.d. | 42 days |
| 4 | 8 | Yes | Compound 1 | 10 | p.o. | q.d. | 42 days |
| 5 | 8 | Yes | Compound 1 | 30 | p.o. | q.d. | 42 days |

Note:
Group 1 was a “healthy control group”, which was fed a standard diet and not injected with DEN.
Vehicle: 40% PEG400/60% Phosal 50 PG

The body weight of animals was measured at the day of the last administration (the end of the test). After the animal were fasted for 6 hours, all animals were euthanatized to collect blood and liver samples for biochemical and histological analysis.

Liver tissue from animals in each group were analyzed for NAS scores, the unweighted sum of the steatosis (0-3), lobular inflammation (0-3) and hepatocellular ballooning (0-2) scores, after HE-staining, and analyzed for liver fibrosis score (0-4) after Sirius red staining.

Liver tissue from animals in each group were analyzed for NAS scores using the methods disclosed above in Example 16. Specifically, the scores assessed were:
1) the unweighted sum of the steatosis (0-3);
2) lobular inflammation (0-3);
3) hepatocellular ballooning (0-2); and
4) liver fibrosis score (0-4), after Sirius red staining.

Prism, version 6, (GraphPad Software Inc., San Diego, CA) was used for all statistical analysis and for graphic presentation.

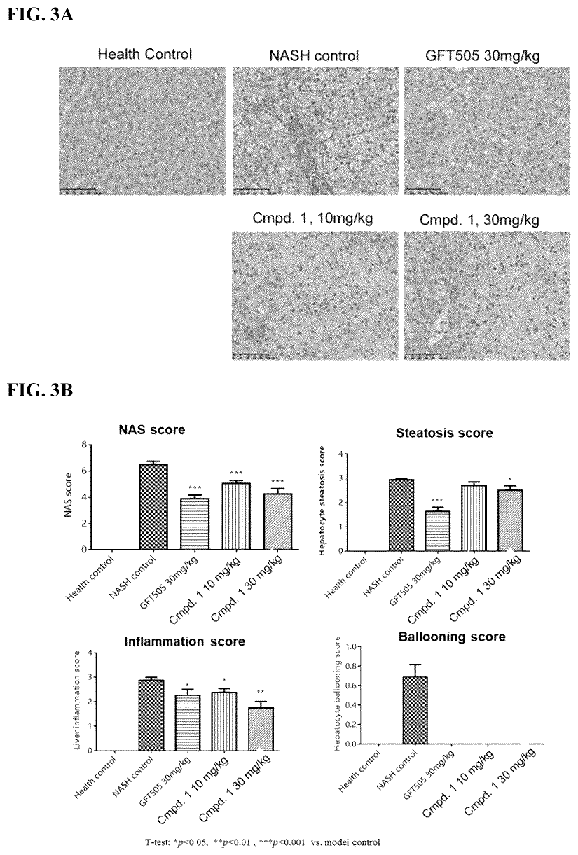

To explore the effect of Compound 1 on NASH and fibrosis, in comparison to a PPARα/δ agonist GFT505, rats with DEN-HFD-CHOL induced non-alcoholic steatohepatitis were treated with Compound 1 at 10 mg/kg and 30 mg/kg (qd, p.o.), and GFT505 at 30 mg/kg (qd, p.o.), as detailed in Table 5. After drug treatment for 42 days, animals were euthanatized to collect liver samples for pathological analysis. HE-staining of liver tissue demonstrated that compared to healthy control, DEN injection and HFD+CHOL diet induced severe steatosis, lobular inflammation and hepatocellular ballooning, all of which were alleviated following treatment with Compound 1 or GFT505 (FIG. 3A). As shown in FIG. 3B, the NAS score was significantly reduced by Compound 1 and GFT505 treatment. Specifically, while both Compound 1 and GFT505 completely abolished ballooning, Compound 1 showed improved anti-inflammation effects but more modest effects on steatosis than GFT505.

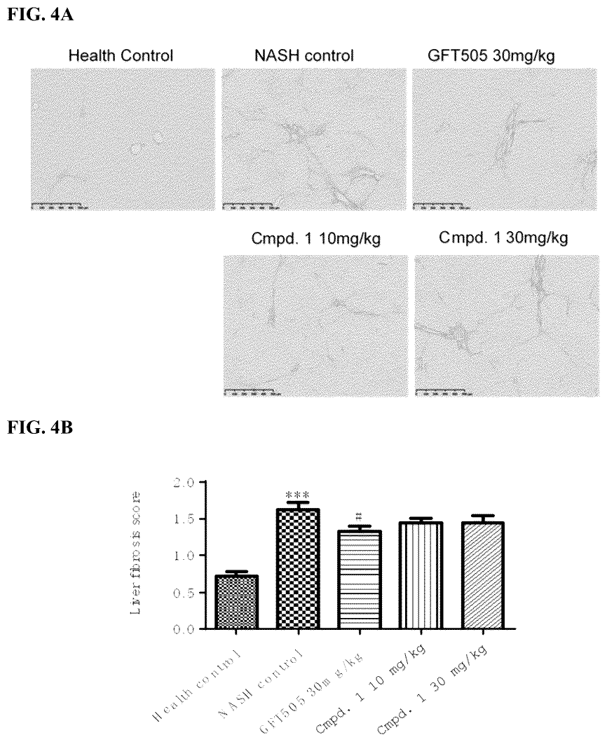

Compound 1 additionally demonstrated modest alleviation of fibrosis, as shown in Sirius red staining of liver tissues (FIG. 4A) and fibrosis score analysis (FIG. 4B).

Collectively, these results suggest that Compound 1 is efficacious for the treatment of non-alcoholic steatohepatitis, based on NAS score alleviation through reduced inflammation, ballooning and steatosis in a rat model of non-alcoholic steatosis. Additionally, Compound 1 has demonstrated potential to alleviate fibrosis in this non-alcoholic steatohepatitis model, underscoring that Compound 1 is useful for the treatment of non-alcoholic steatohepatitis.

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein. Furthermore, the foregoing Description and Examples are exemplary of the present invention and not limiting thereof. The scope of the invention is therefore set out in the appended claims.

All patents and publications cited herein are fully incorporated by reference herein in their entirety.

1. A method of treating non-alcoholic steatohepatitis in a patient comprising administering to the patient in need thereof a therapeutically effective amount of a compound of formula (I): 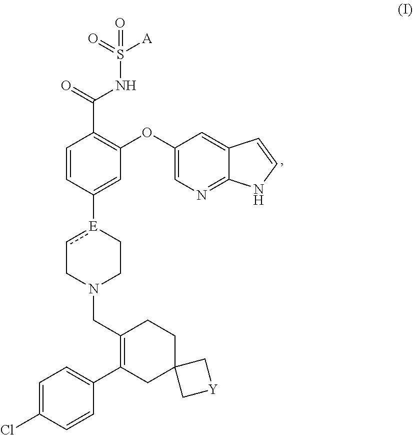
or a pharmaceutically acceptable salt, solvate, hydrate, stereoisomer, or tautomer thereof wherein:
A is selected from the group consisting of: 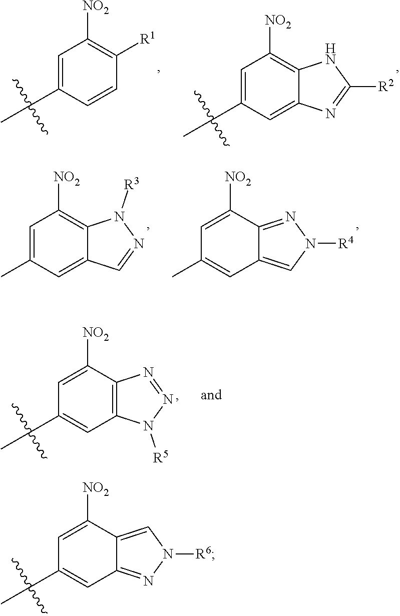
E is selected from the group consisting of:
a carbon atom, wherein [symbol] is a double bond;
—C(H)—, wherein [symbol] is a single bond; and
a nitrogen atom, wherein [symbol] is a single bond;
Y is selected from —C(H)— and —O—;
R¹ is selected from hydrogen and —N(R^{7a})(R^{7b});
R², R³, R⁴, R⁵, and R⁶ are each independently selected from the group consisting of hydrogen, optionally substituted C₁₋₆ alkyl, optionally substituted C₃₋₆ cycloalkyl, heterocyclo, optionally substituted heteroaryl, (heterocyclo)alkyl;
R^{7a} is selected from optionally substituted C₁₋₆ alkyl and optionally substituted (heterocyclo)alkyl; and
R^{7b} is selected from hydrogen and C₁₋₄ alkyl.

2. The method of claim 1, wherein the compound is further given by formula (II): 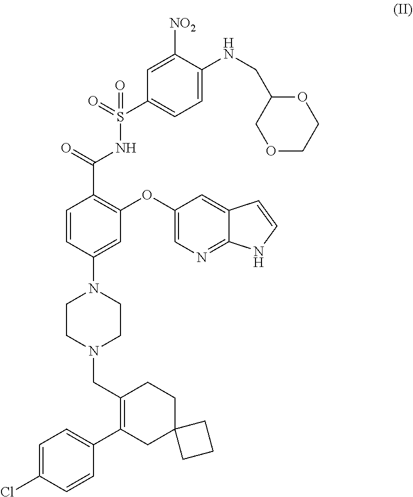 [SMILES: O=C(NS(=O)(=O)c1ccc(NCC2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]
or a pharmaceutically acceptable salt, solvate, hydrate, stereoisomer, or tautomer thereof.

3. The method of claim 2, wherein the compound is selected from the group consisting of: 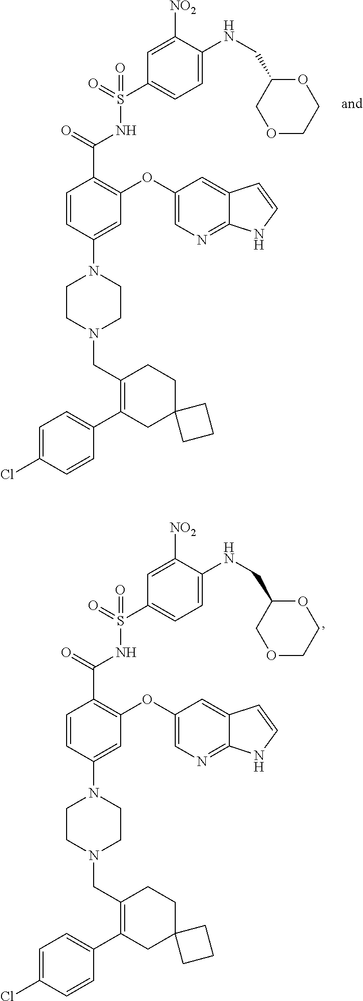 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]
or a pharmaceutically acceptable salt, solvate, hydrate, or tautomer thereof.

4. The method of claim 3, wherein the compound is 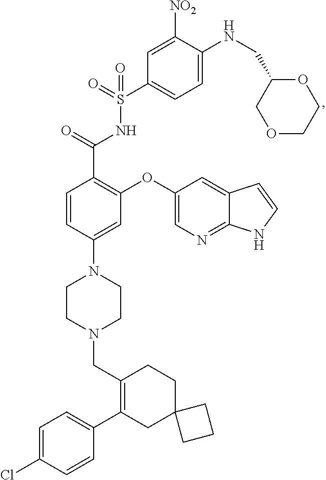 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]
or a pharmaceutically acceptable salt, solvate, hydrate, or tautomer thereof.

5. The method of claim 3, wherein the compound is 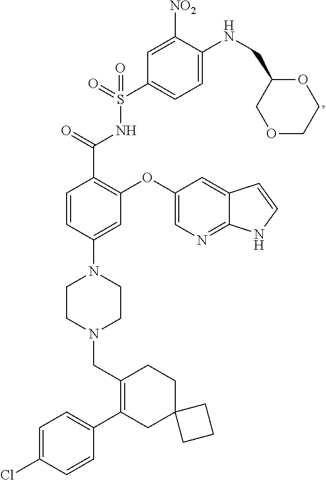 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]
or a pharmaceutically acceptable salt, solvate, hydrate, or tautomer thereof.

6. The method of claim 1, wherein the compound is selected from a group consisting of the compounds recited in Table 1.

7. The method of claim 1, wherein the compound of formula (I) or a pharmaceutically acceptable salt, solvate, hydrate, or tautomer thereof is formulated in a form of a pharmaceutical composition.

8.-12. (canceled)

13. The method of claim 1, wherein the compound of formula (I) is (S)—N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide.

14. The method of claim 1, wherein the patient is diagnosed as having one or more diseases selected from cardiovascular disease, chronic kidney disease, type 2 diabetes mellitus, obesity, and metabolic syndrome.

15. The method of claim 14, wherein the metabolic syndrome is selected from hypertension, hyperglycaemia, hyperlipemia, and insulin resistance (IR).

16. The method of claim 1, wherein the compound of formula (I), or a pharmaceutically acceptable salt, solvate, hydrate, or tautomer thereof, is administered to the patient in need thereof at a dose sufficient to elicit one or more effects selected from the group consisting of reduced liver steatosis, reduced lobular inflammation, reduced hepatocellular ballooning, and reduced liver fibrosis.

17. The method of claim 16, wherein the compound or pharmaceutical composition is administered to the patient in need thereof at a dose sufficient to reduce liver steatosis in the patient.

18. The method of claim 16, wherein the compound or pharmaceutical composition is administered to the patient in need thereof at a dose sufficient to reduce lobular inflammation in the patient.

19. The method of claim 16, wherein the compound or pharmaceutical composition is administered to the patient in need thereof at a dose sufficient to reduce hepatocellular ballooning in the patient.

20. The method of claim 16, wherein the compound or pharmaceutical composition is administered to the patient in need thereof at a dose sufficient to reduce liber fibrosis in the patient.

21. The method of claim 1, further comprising administering to the patient in need thereof a therapeutically effective amount of obeticholic acid.

22. The method of claim 21, wherein the obeticholic acid is administered before the compound of formula (I).

23. The method of claim 21, wherein the obeticholic acid is administered concurrently with the compound of formula (I).

24. The method of claim 21, wherein the obeticholic acid is administered after the compound of formula (I).

25.-52. (canceled)
